(19) United States
(12) Patent Application Publication
(10) Pub. No.: US 2024/0197921 A1
(43) Pub. Date: Jun. 20, 2024

(54) SIGNAL PEPTIDES

(71) Applicant: IMPERIAL COLLEGE INNOVATIONS LIMITED, London (GB)

(72) Inventors: Jack HICKMOTT, London (GB); Eric ALTON, London (GB); Uta GRIESENBACH, London (GB); Possawee PRASERTSUK, London (GB)

(21) Appl. No.: 18/555,385

(22) PCT Filed: Apr. 13, 2022
(86) PCT No.: PCT/GB2022/050929

§ 371 Date: Oct. 13, 2023

(30) Foreign Application Priority Data
Apr. 13, 2021 (GB) 2105277.4

(51) Int. Cl. A61K 48/00; C07K 14/47; C07K 14/535; C07K 14/78; C07K 14/81; C12N 9/16; C12N 9/24; C12N 15/86

(52) U.S. Cl. CPC A61K 48/0066; A61K 48/0033; C07K 14/47; C07K 14/535; C07K 14/78; C07K 14/8125; C12N 9/16; C12N 9/2402; C12N 15/86; C12Y 301/06013; C12Y 302/01018; C07K 2319/02; C12N 2740/15043; C12N 2760/18822

(57) ABSTRACT

The present invention relates to nucleic acid cassettes for gene therapy, particularly to nucleic acid cassettes encoding therapeutic proteins in combination with exogenous signal peptides. The invention further relates to viral and non-viral vectors comprising such nucleic acid cassettes, and the use of such nucleic acid cassettes and vectors to increase expression of therapeutic proteins by airway cells.

### FIELD OF THE INVENTION

The present invention relates to nucleic acid cassettes for gene therapy, particularly to nucleic acid cassettes encoding therapeutic proteins in combination with exogenous signal peptides. The invention further relates to viral and non-viral vectors comprising such nucleic acid cassettes, and the use of such nucleic acid cassettes and vectors to increase expression of therapeutic proteins by airway cells.

### BACKGROUND TO THE INVENTION

The use of nucleic acids as medicine, or gene therapy, is a promising new treatment modality. The reason many gene therapies currently in use or under development are not effective at curing diseases is because it is difficult to make sufficient protein to reach the therapeutic threshold needed to treat or cure the disease. As such, generating sufficient gene expression is a major barrier to the success of many gene therapies.

The current approach to reaching the large doses needed for gene therapy to be successful is to administer massive amounts of the gene therapy to the patient, over 1 trillion viruses per kg of body mass. Producing so much virus is expensive, contributing to the $1,000,000 USD cost of gene therapies, and giving so much virus to a person can trigger immune responses that threaten the health of the patient and the efficacy of the therapy. To circumvent these problems, research to-date has focused on gain of function mutations resulting in more potent proteins. Such an approach has been used previously in the gene therapies for haemophilia B (the Padua mutation in Factor IX) and lipoprotein lipase deficiency (the S447X variant of lipoprotein lipase). However, such gain-of-function mutations are not available for most gene therapies. Furthermore, and even with gain-of-function mutations, high doses of the gene therapy vector were still necessary to make the treatment effective. Therefore, such gain-of-function mutations along do not adequately address the exiting problems associated with producing sufficient quantities of vector, or the unwanted and clinically dangerous side effects associated with the large doses required.

The present inventors have previously developed a lentiviral vector, which has been pseudotyped with hemagglutinin-neuraminidase (HN) and fusion (F) proteins from a respiratory paramyxovirus, comprising a promoter and a transgene. Typically, the backbone of the vector is from a simian immunodeficiency virus (SIV), such as SIV1 or African green monkey SIV (SIV-AGM). Preferably the backbone of a viral vector of the invention is from SIV-AGM. The HN and F proteins function, respectively, to attach to sialic acids and mediate cell fusion for vector entry to target cells. The present inventors discovered that this specifically F/HN-pseudotyped lentiviral vector can efficiently transduce airway epithelium, resulting in transgene expression sustained for periods beyond the proposed lifespan of airway epithelial cells. Importantly, the present inventors also found that re-administration does not result in a loss of efficacy. These features make the vectors of the present invention attractive candidates for treating diseases via their use in expressing therapeutic proteins: (i) within the cells of the respiratory tract; (ii) secreted into the lumen of the respiratory tract; and (iii) secreted into the circulatory system. However, even using this state-of-the-art platform technology, the levels of transgene expressed are at the lower predicted threshold required for clinical efficacy.

There is therefore an unmet clinical need for new technologies to improve the potency of gene therapies. It is an object of the invention to address one or more of these problems. In particular, it is an object of the invention to provide new nucleic acid cassettes and gene therapy vectors which enable increased production of therapeutic proteins, allowing for lower doses of vector to be administered to patients.

### SUMMARY OF THE INVENTION

At present, there remains a pressing need for technology that more efficiently produces therapeutic proteins for gene therapy, including from the inventors' own lentiviral platform. Whilst previous groups have investigated the role of changing signal peptides on protein secretion, there is limited teaching in the art of such changes in the context of gene therapy. Furthermore, no groups to-date have investigated the use of exogenous signal peptides to increase protein expression or secretion in airway cells. Determining suitable exogenous signal peptides is not straightforward. Even considering only naturally occurring signal peptides, 10-20% of the eukaryotic proteome is associated with a signal peptide, and so identifying signal peptides which are particularly effective in increases expression/secretion from airway cells is a significant undertaking.

The present inventors have now shown for the first time that the use of exogenous signal peptides can be used to increase expression and secretion of therapeutic proteins by airway cells. In particular, the present inventors have identified a number of specific signal peptides that have potential utility in increasing expression and secretion of therapeutic proteins by airway cells. Using the exogenous signal peptides of the invention it is possible to produce more protein for every copy of a gene therapy vector or transgene that is put into a cell, increasing the dose of therapeutic protein without increasing the amount of gene therapy vector given to a patient. The inventors' innovative approach has the potential to provide several clinically important advantages: (i) allowing gene therapies to more easily reach therapeutic window, making them more efficacious; (ii) lowering the dose of a gene therapy agent required for administration to a patient, making the gene therapy safer; and/or (iii) lowering the production costs (as less vector is needed per patient), solving a major challenge for clinical trials, & pharmaceutical companies, and health care providers.

Accordingly, the invention provides a nucleic acid cassette comprising: (a) a nucleic acid sequence encoding an exogenous signal peptide; and (b) a nucleic acid sequence encoding a therapeutic protein; wherein the exogenous signal peptide (i) increases secretion of the therapeutic protein from airway cells and/or (ii) increases insertion of the therapeutic protein into the cell membrane of airway cells.

The exogenous signal peptide may be capable of: (a) increasing secretion of the therapeutic protein as compared to secretion of the therapeutic protein without the exogenous signal peptide; and/or (b) increasing secretion of the therapeutic protein as compared to secretion of the therapeutic protein with its endogenous signal peptide. The exogenous signal peptide may be capable of: (a) increasing insertion of the therapeutic protein into the cell membrane of airway cells as compared to membrane insertion of the therapeutic protein without the exogenous signal peptide; and/or (b) increasing insertion of the therapeutic protein into the cell membrane of airway cells as compared to membrane insertion of the therapeutic protein with its endogenous signal peptide.

The exogenous signal peptide may be a signal peptide that drives high secretion from and/or membrane insertion by airway cells, wherein optionally the exogenous signal peptide is selected from: (a) a cartilage acidic protein 1 (CRTAC1) signal peptide; (b) a uteroglobin (SCGB1A1) signal peptide; (c) an alpha-2-macroglobulin (A2M) signal peptide; (d) a synthetic signal peptide; (e) a pulmonary surfactant associated protein A (SFTPA2) signal peptide; (f) a fibronectin (CLEC3B) signal peptide; (g) an alpha-1-antitrypsin (AAT) signal peptide; (h) a granulocyte-macrophage Colony-stimulating factor (GM-CSF) signal peptide; (i) an iduronate 2-sulfatase (IDS) signal peptide; and (j) a hybrid signal peptide, optionally a hybrid of an ATT signal peptide and a synthetic signal peptide. The exogenous signal peptide may comprise or consist of: (a) an amino acid sequence having at least 90% identity to an amino acid selected from the group consisting of: SEQ ID NOs: 1-12; or (b) an amino acid sequence selected from the group consisting of: SEQ ID NOs: 1-12. The nucleic acid sequence encoding the exogenous signal peptide may be 5′ of the nucleic acid sequence encoding the therapeutic protein.

The nucleic acid cassette of the invention may further comprise a promoter configured to express the nucleic acid sequence encoding the exogenous signal peptide and the therapeutic protein. The promoter may be selected from the group consisting of a hybrid human cytomegalovirus (CMV) enhancer/elongation factor 1 a (EF1 a) promoter (hCEF), a CMV promoter and an EF1 a promoter, preferably a hCEF promoter.

The nucleic acid cassette of the invention may further comprise (a) a translation initiation sequence; and/or (b) an internal ribosome entry sequence (IRES).

The therapeutic protein may be (a) a secreted therapeutic protein selected from: AAT, Factor VIII, Surfactant Protein B (SP-B), Factor VII, Factor IX, Factor X, Factor XI, van Willebrand Factor, Granulocyte-Macrophage Colony-Stimulating Factor (GM-CSF), Surfactant Protein C (SP-C), decorin, an anti-inflammatory protein (e.g. IL-10 or TGFβ) or monoclonal antibody, an anti-inflammatory decoy, or a monoclonal antibody against an infectious agent; or (b) CFTR, TRIM72, CSF2RA, ATP-binding cassette sub-family A member 3 (ABCA3) or CSF2RB.

The airway cells may be: (a) lung cells; and/or (b) selected from epithelial cells, basal cells, submucosal gland duct cells, club cells, neuroendocrine cells, bronchoalveolar stem cells, submucosal acinar cells, ionocytes, type I pneumocytes and/or type II pneumocytes.

The invention further provides a gene therapy vector, comprising a nucleic acid cassette of the invention.

Said gene therapy vector may be a non-viral vector, wherein optionally: (a) the non-viral vector is a plasmid; and/or (b) the non-viral vector is comprised in a cationic liposome, which preferably comprises GL67A.

Said gene therapy vector may be a viral vector, optionally selected from: (a) a lentiviral vector; (b) an AAV vector; (c) an adenoviral vector; and (d) a sendai virus vector. The gene therapy vector may be a lentiviral vector that is pseudotyped with hemagglutinin-neuraminidase (HN) and fusion (F) proteins from a respiratory paramyxovirus. The respiratory paramyxovirus may be a Sendai virus. The lentiviral vector may be selected from the group consisting of a Human immunodeficiency virus (HIV) vector, a Simian immunodeficiency virus (SIV) vector, a Feline immunodeficiency virus (FIV) vector, an Equine infectious anaemia virus (EIAV) vector, and a Visna/maedi virus vector. The lentiviral vector may be a SIV vector.

The invention also provides a method of expressing a secreted therapeutic protein in a target cell, comprising delivering a nucleic acid cassette of the invention or a gene therapy vector of the invention into the target cells. Said delivering may comprise integrating said nucleic acid cassette or gene therapy vector into said target cell's genome.

The invention also provides a gene therapy vector of the invention for use in a method of treating a disease. The disease may be a genetic disease. The disease may be (a) a respiratory disease, particularly a genetic respiratory disease; or (b) a cardiovascular disease or blood disorder, particularly a genetic cardiovascular disease or blood disorder. The disease may be selected from cystic fibrosis (CF); Primary Ciliary Dyskinesia (PCD); Surfactant Protein B (SP-B) Deficiency; Alpha 1-antitrypsin Deficiency (A1AD); Pulmonary Alveolar Proteinosis (PAP); Chronic obstructive pulmonary disease (COPD); Pulmonary surfactant metabolism dysfunction 2 (SMDP2); Pulmonary surfactant metabolism dysfunction 3 (SMDP3); Acute respiratory distress syndrome (ARDS); COVID-19; a pulmonary fibrotic disease; a pulmonary allergic condition; a pulmonary bacterial infection; lung cancer; a dysplastic change in the lungs; and haemophilia.

The invention further provides a cell comprising a nucleic acid cassette of the invention or a gene therapy vector of the invention.

The invention also provides a composition comprising a nucleic acid cassette of the invention or a gene therapy vector of the invention and a pharmaceutically acceptable carrier, diluent or excipient.

### BRIEF DESCRIPTION OF THE DRAWINGS

FIG. 1: Seven signal peptides identified by bioinformatic analysis as strong drivers of protein secretion within the lungs.

FIG. 2: Alpha-2-Macroglobulin (A2M) Signal Peptide Design. A) To isolate the signal peptide from A2M, the first 50 amino acids of the protein sequence were analysed using SignalP-5.0 and a strong cleavage site was identified at alanine 23. B) The first 23 amino acids of the A2M protein were reverse translated and codon optimized for translation in human cells. The resulting nucleic acid sequence can then be fused to the coding sequence of a secreted protein.

FIG. 3: Signal Peptides were cloned into Lentiviral transfer plasmids. A) Signal peptides were cloned into a lentiviral transfer plasmid containing a therapeutic protein (GM-CSF) and an expression cassette. B) Clones were screened by a HindIII restriction digest, producing two bands as expected for all plasmids. MW-NEB 1 KB ladder, 44—pIC044 (GM-CSF signal peptide), 77-1 & 77-2—pIC077 clones 1 & 2 (A2M signal peptide), 78-1 & 78-2—clones 1 & 2 (CLEC3B signal peptide), 79-1 & 79-2—clones 1 & 2 (CRTAC1 signal peptide), 80-1 & 80-2—pIC080 clones 1 & 2 (SCGB1A1 signal peptide), and 81-1—pIC081 clone 1 (SFTPA1 signal peptide).

FIG. 4: Proper fusion of the signal peptides to the therapeutic transgene (GM-CSF) was confirmed by sanger sequencing providing 2-fold coverage of the signal peptide and fusion site with GM-CSF.

FIG. 5: Schematics of signal peptides (lime) fused to the N-terminal of AAT (green). A) Endogenous AAT signal Peptide (AAT.SP), B) Synthetic Secrecon Signal peptide (Secrecon.SP), C) hybrid of endogenous signal peptide and Secrecon (Hybrid.SP), and D) Iduronate 2-Sulfatase signal peptide (IDS.SP).

FIG. 6: Sequence of Lung Signal Peptides Fused to GM-CSF

FIG. 7: Secrecon signal peptide increases AAT secretion in HEK293 cells. HEK293 cells were transfected with lentiviral transfer plasmids encoding the AAT gene fused to the AAT, secrecon, hybrid, or IDS signal peptides. In HEK293 cells exchanging the AAT signal peptide for the secrecon signal peptide increased the amount of AAT secreted into the tissue culture media (median values of 0.39 pg/cell and 0.84 pg/cell for AAT and secrecon signal peptides respectively).

FIG. 8: Lung signal peptides modify GM-CSF secretion from transfected HEK293T Cells (grey and black dots represent first and second experiments respectively, each dot represents a measurement made from a different well of transduced cells). Bars represent median values. Data was analysed with a Kruskal-Wallis test comparing all groups except NTC (non-transduced control).

FIG. 9: Lung signal peptides modify GM-CSF secretion from human air liquid interface cultures transduced with VSV-G pseudotyped lentivirus (grey and black dots represent first and second experiments respectively, each dot represents a measurement made from a different ALI). Bars represent median values. Dotted line represents the mean amount of GM-CSF secreted using the native signal peptide. Data analysed by comparing signal peptides to native with a Kruskal-Wallace test and Dunn's multiple test correction, ** p<0.005.

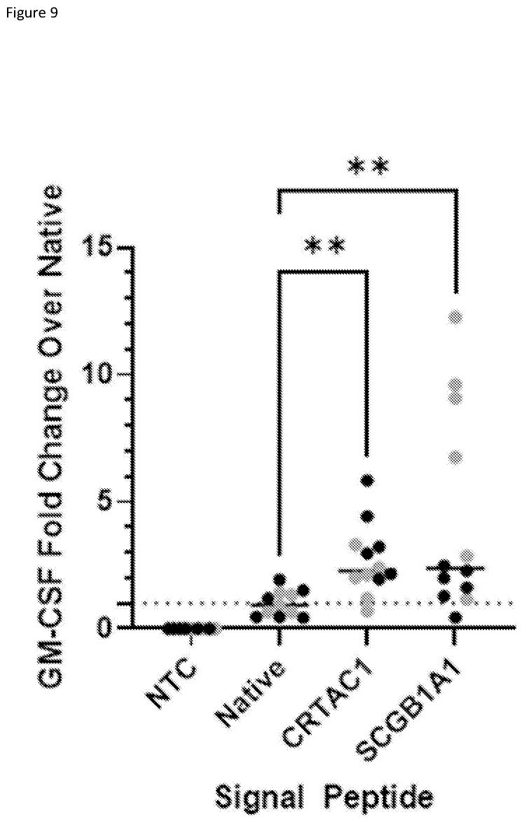

FIG. 10: SCGB1A1 signal peptide increased AAT secretion from human air liquid interface cultures transduced with VSV-G pseudotyped lentivirus (each dot represents a measurement made from a different ALI). Bars represent median values. Dotted line represents the median amount of AAT secreted by non-transduced ALIs. Native and SCGB1A1 compared using a one-tailed Mann-Whitney test.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

As used herein the terms “signal peptide”, “signal sequence”, “targeting sequence”, “leader sequence” and “secretory signal” are used interchangeably to mean heterogenous peptide sequences that are found at the N-terminus of secreted proteins that are instrumental in initiating the secretion process. In particular, signal peptides are found in proteins that are targeted to the endoplasmic reticulum and eventually destined to be either secreted or retained in the cell membrane of the cell, particularly as single-pass membrane proteins. Signal peptides are typically removed to produce the mature form of the protein. Signal peptides are normally short peptides, typically about 5 to about 40 amino acids in length, such as about 5 to about 35, or about 10 to about 35 amino acids in length, preferably about 10 to about 30 or about 15 to about 30 amino acids in length. A signal peptide may comprise a core of hydrophobic amino acids, said core typically being about 4 to about 20, such as about 5 to about 20, about 5 to about 16 or about 5 to about 15 amino acids in length). When present, a signal peptide is typically present at the N-terminus of a protein.

A “vector” or “construct” (sometimes referred to as gene delivery or gene transfer “vehicle”) refers to a macromolecule or complex of molecules comprising a polynucleotide to be delivered to a host cell, either in vitro or in vivo. A vector can be a linear or a circular molecule. A vector of the invention may be viral or non-viral. All disclosure herein in relation vectors of the invention applies equally to viral and non-viral vectors unless otherwise stated. All disclosure in relation to viral vectors of the invention applies equally and without reservation to lentiviral (e.g. SIV) vectors, particularly to lentiviral (e.g. SIV) vectors that are pseudotyped with hemagglutinin-neuraminidase (HN) and fusion (F) proteins from a respiratory paramyxovirus (also referred to herein as SIV F/HN or SIV-FHN).

As used herein, the term “plasmid”, refers to a common type of non-viral vector. A plasmid is an extra-chromosomal DNA molecule separate from the chromosomal DNA which is capable of replicating independently of the chromosomal DNA. Preferably a plasmid is circular and may be double-stranded.

The terms “nucleic acid cassette”, “nucleic acid construct”, “expression cassette” and “nucleic acid expression cassette” are used interchangeably to mean a nucleic acid molecule that is capable of directing transcription. A nucleic acid cassette includes, at the least, a promoter or a structure functionally equivalent to a promoter and a nucleic acid sequence to be transcribed. Thus, a nucleic acid cassette includes, at the least, a promoter or a structure functionally equivalent to a promoter and a nucleic acid sequence encoding a protein of interest. In the present invention, a nucleic acid cassette includes, at the least, a promoter or a structure functionally equivalent to a promoter, a nucleic acid sequence encoding a signal peptide and a nucleic acid encoding a therapeutic protein. A nucleic acid cassette may include additional elements, such as an enhancer, and/or a transcription termination signal.

As used herein, the terms “transduced” and “modified” are used interchangeably to describe cells which have been modified to express a transgene of interest. Typically the modification occurs through transduction of the cells.

The term “exogenous,” when used in relation to a signal peptide refers to a signal peptide which has been linked with a therapeutic protein and/or introduced to an expression cassette by artificial means. An exogenous signal peptide may be from a different organism or cell to the therapeutic protein. An exogenous signal peptide may be from the same organism, but naturally associated with a different protein than the therapeutic protein with which it is linked in the present invention.

As used herein, the terms “titre” and “yield” are used interchangeably to mean the amount of viral (e.g. lentiviral, particularly SIV) vector produced by a method of the invention. Titre is the primary benchmark characterising manufacturing efficiency, with higher titres generally indicating that more vector is manufactured (e.g. using the same amount of reagents). Titre or yield may relate to the number of vector genomes that have integrated into the genome of a target cell (integration titre), which is a measure of “active” virus particles, i.e. the number of particles capable of transducing a cell. Transducing units (TU/mL also referred to as TTU/mL) is a biological readout of the number of host cells that get transduced under certain tissue culture/virus dilutions conditions, and is a measure of the number of “active” virus particles. The total number of (active+inactive) virus particles may also be determined using any appropriate means, such as by measuring either how much Gag is present in the test solution or how many copies of viral RNA are in the test solution. Assumptions are then made that a viral (e.g. lentivirus, particularly SIV) particle contains either 2000 Gag molecules or 2 viral RNA molecules. Once total particle number and a transducing titre/TU have been measured, a particle:infectivity ratio calculated.

Amino acids are referred to herein using the name of the amino acid, the three-letter abbreviation or the single letter abbreviation.

Unless otherwise indicated, any nucleic acid sequences are written left to right in 5′ to 3′ orientation; amino acid sequences are written left to right in amino to carboxy orientation, respectively.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides. The terms “transgene” and “gene” are also used interchangeably and both terms encompass fragments or variants thereof encoding the target protein.

The transgenes of the present invention include nucleic acid sequences that have been removed from their naturally occurring environment, recombinant or cloned DNA isolates, and chemically synthesized analogues or analogues biologically synthesized by heterologous systems.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, lie, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide. For example, a fragment may comprise at least 5, at least 10, at least 11, at least 12, at least 13, at least 14, at least 15, at least 16, at least 17, at least 18, at least 19, at least 20 or more amino acids from a signal peptide of the invention. A fragment may be continuous or discontinuous, preferably continuous.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino | | Degenerate |
|---|---|---|
| Acid | Codons | Codon |
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species. Thus, according to the invention, in addition to the gag-pol genes any nucleic acid sequence may be codon-optimised for expression in a host or target cell. In particular, the vector genome (or corresponding plasmid), the REV gene (or corresponding plasmid), the fusion protein (F) gene (or correspond plasmid) and/or the hemagglutinin-neuraminidase (HN) gene (or corresponding plasmid, or any combination thereof may be codon-optimised.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, at least about 95%, or at least about 98%, or at least about 99%, or at least about 100%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level. In the context of a yield or titre, an “increase” is an observable or statistically significant increase in such level.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In a preferred embodiment, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications or symptoms related to such a condition, and optionally, have already undergone treatment for a condition as defined herein or the one or more complications or symptoms related to said condition. Alternatively, a subject can also be one who has not been previously diagnosed as having a condition as defined herein or one or more or symptoms or complications related to said condition. For example, a subject can be one who exhibits one or more risk factors for a condition, or one or more or symptoms or complications related to said condition or a subject who does not exhibit risk factors.

As used herein, the term “healthy individual” refers to an individual or group of individuals who are in a healthy state, e.g. individuals who have not shown any symptoms of the disease, have not been diagnosed with the disease and/or are not likely to develop the disease e.g. cystic fibrosis (CF) or any other disease described herein). Preferably said healthy individual(s) is not on medication affecting CF and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia

The publications discussed herein are provided solely for their disclosure prior to the filing date of the present application. Nothing herein is to be construed as an admission that such publications constitute prior art to the claims appended hereto.

Disclosure related to the various methods of the invention are intended to be applied equally to other methods, therapeutic uses or methods, the data storage medium or device, the computer program product, and vice versa.

### Signal Peptides

The present invention relates to signal peptides that are effective at enhancing the expression of proteins (e.g. therapeutic proteins as described herein) by lung cells and/or airway cells. The signal peptides of the invention have typically been identified through bioinformatic search as driving high expression from the lungs and airways, or are synthetic signal peptides designed to drive high expression in numerous cell & tissue types, particularly airway cells. In particular, the signal peptides of the invention are effective at increasing the secretion of therapeutic proteins from airway cells and/or increasing insertion of therapeutic proteins into the cell membrane of airway cells (as described herein).

The signal peptides of the invention are exogenous. As used herein, an exogenous signal peptide is one that is not associated with the therapeutic protein as normally expressed in a cell or patient. In other words, the signal peptide is not the endogenous or native signal peptide for any given protein (e.g. therapeutic protein). Whilst a signal peptide may be an endogenous signal peptide for a protein, it will not be used in combination with its endogenous protein in the nucleic acid cassettes of the invention. By way of non-limiting example, the Serpin Family A Member 1 (SERPINA1) gene has an endogenous signal peptide, the AAT signal peptide. When a nucleic acid cassette of the invention comprises a nucleic acid sequence encoding the AAT protein, the signal peptide will not be the AAT signal peptide, but rather an exogenous signal peptide as described herein (e.g. a CRTAC1 signal peptide or a CLEC3B signal peptide). In contrast, a nucleic acid cassette of the invention which comprises a nucleic acid encoding for a non-AAT transgene (e.g. CFTR), the AAT signal peptide may be used as the exogenous signal peptide. By way of a further non-limiting example, when a nucleic acid cassette of the invention comprises a nucleic acid sequence encoding the GM-CSF protein, the signal peptide will not be the GM-CSF signal peptide, but rather an exogenous signal peptide as described herein (e.g. a CRTAC1 signal peptide or a AAT signal peptide). In contrast, a nucleic acid cassette of the invention which comprises a nucleic acid encoding for a non-GM-CSF transgene (e.g. FVIII or IL-10), the GM-CSF signal peptide may be used as the exogenous signal peptide.

The present inventors have identified numerous signal peptides that are effective at enhancing the expression, secretion and/or membrane insertion of proteins (e.g. therapeutic proteins as described herein) by airway cells. In particular, the present inventors have identified a cartilage acidic protein 1 (CRTAC1) signal peptide; a uteroglobin (SCGB1A1) signal peptide; an alpha-2-macroglobulin (A2M) signal peptide; a pulmonary surfactant associated protein A (SFTPA2) signal peptide; a fibronectin (CLEC3B) signal peptide; an alpha-lantitrypsin (AAT) signal peptide; a granulocyte-macrophage Colony-stimulating factor (GM-CSF) signal peptide; and an iduronate 2-sulfatase (IDS) signal peptide as exogenous signal peptides that may be used in nucleic acid cassettes of the invention to increase the expression, secretion and/or membrane insertion of a therapeutic protein as described herein.

A CRTAC1 signal peptide may comprise or consist of an amino acid sequence corresponding to amino acid residues 1 to 27 of UniProt Accession No. Q9NQ79 (accessed 25 Mar. 2021). A CRTAC1 signal peptide may comprise or consist of the amino acid sequence MAPSADPGMSRMLPFLLLLWFLPITEG (SEQ ID NO: 1). Variants of amino acid residues 1 to 27 of UniProt Accession No. Q9NQ79 and/or SEQ ID NO: 1 are also included (encompassing all variants as described herein), particularly variants with at least 90% (such as at least 90, 92, 94, 95, 96, 97, 98, 99 or 100%) to amino acid residues 1 to 27 of UniProt Accession No. Q9NQ79 and/or SEQ ID NO: 1. Fragments of amino acid residues 1 to 27 of UniProt Accession No. Q9NQ79 and/or SEQ ID NO: 1 (encompassing all fragments as described herein) are also included, particularly fragments of at least 10, at least 15 or at least 20 (consecutive or non-consecutive, preferably consecutive) amino acids of amino acid residues 1 to 27 of UniProt Accession No. Q9NQ79 and/or SEQ ID NO: 1. Preferably a CRTAC1 signal peptide may comprise or consist of SEQ ID NO: 1, even more preferably may consist of SEQ ID NO: 1.

An AAT signal peptide may comprise or consist of an amino acid sequence corresponding to amino acid residues 1 to 24 of UniProt Accession No. P01009 (accessed 25 Mar. 2021). An AAT signal peptide may comprise or consist of the amino acid sequence MPSSVSWGILLLAGLCCLVPVSLA (SEQ ID NO: 2). Variants of amino acid residues 1 to 24 of UniProt Accession No. P01009 and/or SEQ ID NO: 2 are also included (encompassing all variants as described herein), particularly variants with at least 90% (such as at least 90, 92, 94, 95, 96, 97, 98, 99 or 100%) to amino acid residues 1 to 24 of UniProt Accession No. P01009 and/or SEQ ID NO: 2. Fragments of amino acid residues 1 to 24 of UniProt Accession No. P01009 and/or SEQ ID NO: 2 (encompassing all fragments as described herein) are also included, particularly fragments of at least 10, at least 15 or at least 20 (consecutive or non-consecutive, preferably consecutive) amino acids of amino acid residues 1 to 24 of UniProt Accession No. P01009 and/or SEQ ID NO: 2. Preferably an AAT signal peptide may comprise or consist of SEQ ID NO: 2, even more preferably may consist of SEQ ID NO: 2.

A CLEC3B signal peptide may comprise or consist of an amino acid sequence corresponding to amino acid residues 1 to 21 of UniProt Accession No. P05452 (accessed 25 Mar. 2021). A CLEC3B signal peptide may comprise or consist of the amino acid sequence MELWGAYLLLCLFSLLTQVTT (SEQ ID NO: 3). Variants of amino acid residues 1 to 21 of UniProt Accession No. P05452 and/or SEQ ID NO: 3 are also included (encompassing all variants as described herein), particularly variants with at least 90% (such as at least 90, 92, 94, 95, 96, 97, 98, 99 or 100%) to amino acid residues 1 to 21 of UniProt Accession No. P05452 and/or SEQ ID NO: 3. Fragments of amino acid residues 1 to 21 of UniProt Accession No. P05452 and/or SEQ ID NO: 3 (encompassing all fragments as described herein) are also included, particularly fragments of at least 10, at least 15 or at least 20 (consecutive or non-consecutive, preferably consecutive) amino acids of amino acid residues 1 to 21 of UniProt Accession No. P05452 and/or SEQ ID NO: 3. Preferably a CLEC3B signal peptide may comprise or consist of SEQ ID NO: 3, even more preferably may consist of SEQ ID NO: 3.

A SCGB1A1 signal peptide may comprise or consist of an amino acid sequence corresponding to amino acid residues 1 to 21 of UniProt Accession No. P11684 (accessed 25 Mar. 2021). A SCGB1A1 signal peptide may comprise or consist of the amino acid sequence MKLAVTLTLVTLALCCSSASA (SEQ ID NO: 5). Variants of amino acid residues 1 to 21 of UniProt Accession No. P11684 and/or SEQ ID NO: 5 are also included (encompassing all variants as described herein), particularly variants with at least 90% (such as at least 90, 92, 94, 95, 96, 97, 98, 99 or 100%) to amino acid residues 1 to 21 of UniProt Accession No. P11684 and/or SEQ ID NO: 5. Fragments of amino acid residues 1 to 21 of UniProt Accession No. P11684 and/or SEQ ID NO: 5 (encompassing all fragments as described herein) are also included, particularly fragments of at least 10, at least 15 or at least 20 (consecutive or non-consecutive, preferably consecutive) amino acids of amino acid residues 1 to 21 of UniProt Accession No. P11684 and/or SEQ ID NO: 5. Preferably a SCGB1A1 signal peptide may comprise or consist of SEQ ID NO: 5, even more preferably may consist of SEQ ID NO: 5.

An A2M signal peptide may comprise or consist of an amino acid sequence corresponding to amino acid residues 1 to 23 of UniProt Accession No. P01023 (accessed 25 Mar. 2021). An A2M signal peptide may comprise or consist of the amino acid sequence MGKNKLLGPSLVLLLLVLLPTDA (SEQ ID NO: 4). Variants of amino acid residues 1 to 23 of UniProt Accession No. P01023 and/or SEQ ID NO: 4 are also included (encompassing all variants as described herein), particularly variants with at least 90% (such as at least 90, 92, 94, 95, 96, 97, 98, 99 or 100%) to amino acid residues 1 to 23 of UniProt Accession No. P01023 and/or SEQ ID NO: 4. Fragments of amino acid residues 1 to 23 of UniProt Accession No. P01023 and/or SEQ ID NO: 4 (encompassing all fragments as described herein) are also included, particularly fragments of at least 10, at least 15 or at least 20 (consecutive or non-consecutive, preferably consecutive) amino acids of amino acid residues 1 to 21 of UniProt Accession No. P01023 and/or SEQ ID NO: 4. Preferably an A2M signal peptide may comprise or consist of SEQ ID NO: 4, even more preferably may consist of SEQ ID NO: 4.

A SFTPA2 signal peptide may comprise or consist of an amino acid sequence corresponding to amino acid residues 1 to 20 of UniProt Accession No. Q8IWL1 (accessed 25 Mar. 2021). A SFTPA2 signal peptide may comprise or consist of the amino acid sequence MWLCPLALNLILMAASGAAC (SEQ ID NO: 6). Variants of amino acid residues 1 to 20 of UniProt Accession No. Q8IWL1 and/or SEQ ID NO: 6 are also included (encompassing all variants as described herein), particularly variants with at least 90% (such as at least 90, 92, 94, 95, 96, 97, 98, 99 or 100%) to amino acid residues 1 to 20 of UniProt Accession No. Q8IWL1 and/or SEQ ID NO: 6. Fragments of amino acid residues 1 to 20 of UniProt Accession No. Q8IWL1 and/or SEQ ID NO: 6 (encompassing all fragments as described herein) are also included, particularly fragments of at least 10 or at least 15 or (consecutive or non-consecutive, preferably consecutive) amino acids of amino acid residues 1 to 20 of UniProt Accession No. Q8IWL1 and/or SEQ ID NO: 6. Preferably a SFTPA2 signal peptide may comprise or consist of SEQ ID NO: 6, even more preferably may consist of SEQ ID NO: 6.

A GM-CSF signal peptide may be a mouse of human GM-CSF signal peptide, with a human GM-CSF signal peptide being preferred. A human GM-CSF signal peptide may comprise or consist of an amino acid sequence corresponding to amino acid residues 1 to 17 of UniProt Accession No. P04141 (accessed 25 Mar. 2021). A (human) GM-CSF signal peptide may comprise or consist of the amino acid sequence MWLQSLLLLGTVACSIS (SEQ ID NO: 7). Variants of amino acid residues 1 to 17 of UniProt Accession No. P04141 and/or SEQ ID NO: 7 are also included (encompassing all variants as described herein), particularly variants with at least 90% (such as at least 90, 92, 94, 95, 96, 97, 98, 99 or 100%) to amino acid residues 1 to 17 of UniProt Accession No. P04141 and/or SEQ ID NO: 7. Fragments of amino acid residues 1 to 17 of UniProt Accession No. P04141 and/or SEQ ID NO: 7 (encompassing all fragments as described herein) are also included, particularly fragments of at least 10 or at least 15 (consecutive or non-consecutive, preferably consecutive) amino acids of amino acid residues 1 to 17 of UniProt Accession No. P04141 and/or SEQ ID NO: 7. Preferably a GM-CSF signal peptide may comprise or consist of SEQ ID NO: 7, even more preferably may consist of SEQ ID NO: 7. A (mouse) GM-CSF signal peptide may comprise or consist of the amino acid sequence MWLQNLLFLGIVVYSLS (SEQ ID NO: 8). Variants of amino acid residues 1 to 17 of UniProt Accession No. P01587 and/or SEQ ID NO: 8 are also included (encompassing all variants as described herein), particularly variants with at least 90% (such as at least 90, 92, 94, 95, 96, 97, 98, 99 or 100%) to amino acid residues 1 to 17 of UniProt Accession No. P01587 and/or SEQ ID NO: 8. Fragments of amino acid residues 1 to 17 of UniProt Accession No. P01587 and/or SEQ ID NO: 8 (encompassing all fragments as described herein) are also included, particularly fragments of at least 10 or at least 15 (consecutive or non-consecutive, preferably consecutive) amino acids of amino acid residues 1 to 17 of UniProt Accession No. P01587 and/or SEQ ID NO: 8. A mouse GM-CSF signal peptide may comprise or consist of SEQ ID NO: 8, more preferably may consist of SEQ ID NO: 8.

An IDS signal peptide may comprise or consist of an amino acid sequence corresponding to amino acid residues 1 to 25 of UniProt Accession No. P22304 (accessed 25 Mar. 2021). An IDS signal peptide may comprise or consist of the amino acid sequence MPPPRTGRGLLWLGLVLSSVCVALGA (SEQ ID NO: 9). Variants of amino acid residues 1 to 25 of UniProt Accession No. P22304 and/or SEQ ID NO: 9 are also included (encompassing all variants as described herein), particularly variants with at least 90% (such as at least 90, 92, 94, 95, 96, 97, 98, 99 or 100%) to amino acid residues 1 to 25 of UniProt Accession No. P22304 and/or SEQ ID NO: 9. Fragments of amino acid residues 1 to 25 of UniProt Accession No. P22304 and/or SEQ ID NO: 9 (encompassing all fragments as described herein) are also included, particularly fragments of at least 10, at least 15 or at least 20 (consecutive or non-consecutive, preferably consecutive) amino acids of amino acid residues 1 to 25 of UniProt Accession No. P22304 and/or SEQ ID NO: 9. Preferably an IDS signal peptide may comprise or consist of SEQ ID NO: 9, even more preferably may consist of SEQ ID NO: 9.

An exogenous signal peptide for use according to the present invention may be a synthetic signal peptide. As used herein the term “synthetic signal peptide” is used to describe a signal peptide which is not naturally occurring (e.g. in eukaryotes such as human or non-human animals, plants, or yeast, and/or prokaryotes) and typically excludes variants or derivatives of naturally occurring signal peptides. Synthetic signal peptides of the invention may be designed to further optimise the expression, secretion and/or membrane insertion of proteins (e.g. therapeutic proteins as described herein) by airway cells. By way of non-limiting example, a synthetic signal peptide of the invention may be a Secrecon signal peptide.

A Secrecon signal peptide may comprise or consist of the amino acid sequence MWWRLWWLLLLLLLLWPMVWA (SEQ ID NO: 10) or MWWRLWWLLLLLLLLWPMVWAAA (SEQ ID NO: 11). Variants of SEQ ID NO: 10 or 11 are also included (encompassing all variants as described herein), particularly variants with at least 90% (such as at least 90, 92, 94, 95, 96, 97, 98, 99 or 100%) to SEQ ID NO: 10 or 11. Fragments of SEQ ID NO: 10 or 11 (encompassing all fragments as described herein) are also included, particularly fragments of at least 10, at least 15 or at least 20 (consecutive or non-consecutive, preferably consecutive) amino acids of SEQ ID NO: 10 or 11. Preferably a Secrecon signal peptide may comprise or consist of SEQ ID NO: 10 or 11, even more preferably may consist of SEQ ID NO: 10 or 11.

An exogenous signal peptide for use according to the present invention may be a hybrid signal peptide. As used herein the term “hybrid signal peptide” is used to describe a signal peptide which comprises or consists of amino acid sequences from two or more (naturally occurring and/or synthetic) signal peptides. A hybrid signal peptide of the invention may comprise or consist of amino acid sequences from two or more naturally occurring signal peptides. Alternatively, a hybrid signal peptide may comprise or consist of amino acid sequences from a naturally occurring signal peptide and a synthetic signal peptide. Hybird signal peptides of the invention may be designed to further optimise the expression, secretion and/or membrane insertion of proteins (e.g. therapeutic proteins as described herein) by airway cells compared with the signal peptides from which they are derived.

By way of non-limiting example, a hybrid signal peptide of the invention may comprise an amino acid sequence from a Secrecon signal peptide and an amino acid sequence from a CRTAC1 signal peptide (also referred to as a Secrecon/CRTAC1 signal peptide or a hybrid Secrecon/CRTAC1 signal peptide); an AAT signal peptide (also referred to as a Secrecon/AAT signal peptide or a hybrid Secrecon/AAT signal peptide); a CLEC3B signal peptide (also referred to as a Secrecon/CLEC3B signal peptide or a hybrid Secrecon/CLEC3B signal peptide); an A2M signal peptide (also referred to as a Secrecon/A2M signal peptide or a hybrid Secrecon/A2M signal peptide); a SCGB1A1 signal peptide (also referred to as a Secrecon/SCGB1A1 signal peptide or a hybrid Secrecon/SCGB1A1 signal peptide); a SFTPA2 signal peptide (also referred to as a Secrecon/SFTPA2 signal peptide or a hybrid Secrecon/SFTPA2 signal peptide); a GM-CSF signal peptide (also referred to as a Secrecon/GM-CSF signal peptide or a hybrid Secrecon/GM-CSF signal peptide); or an IDS signal peptide (also referred to as a Secrecon/IDS signal peptide or a hybrid Secrecon/IDS signal peptide); as described herein.

A preferred hybrid signal peptide of the invention may comprise an amino acid sequence from a Secrecon signal peptide and an amino acid sequence from an AAT signal peptide as defined herein (i.e. a Secrecon-AAT hybrid signal peptide). For example, such a Secrecon-AAT hybrid signal peptide may comprise or consist of: MPWWVSWWLLLLLLLCCLVPVVWAAA (SEQ ID NO: 12). Variants of SEQ ID NO: 12 are also included (encompassing all variants as described herein), particularly variants with at least 90% (such as at least 90, 92, 94, 95, 96, 97, 98, 99 or 100%) to SEQ ID NO: 12. Fragments of SEQ ID NO: 12 (encompassing all fragments as described herein) are also included, particularly fragments of at least 10, at least 15 or at least 20 (consecutive or non-consecutive, preferably consecutive) amino acids of SEQ ID NO: 12. Preferably a Secrecon-AAT hybrid signal peptide may comprise or consist of SEQ ID NO: 12, even more preferably may consist of SEQ ID NO: 12.

The exogenous signal peptide of the invention may be a CRTAC1 signal peptide; an SCGB1A1 signal peptide; an A2M signal peptide; a Secrecon signal peptide; an SFTPA2 signal peptide; a CLEC3B signal peptide; an AAT signal peptide; a GM-CSF signal peptide; an IDS signal peptide or a hybrid signal peptide. The exogenous signal peptide of the invention may be a CRTAC1 signal peptide; an SCGB1A1 signal peptide; an A2M signal peptide; a Secrecon signal peptide; an SFTPA2 signal peptide; a CLEC3B signal peptide; an AAT signal peptide; or a GM-CSF signal peptide. The exogenous signal peptide of the invention may be a CRTAC1 signal peptide; an SCGB1A1 signal peptide; an A2M signal peptide; a Secrecon signal peptide; an SFTPA2 signal peptide; or a CLEC3B signal peptide. The exogenous signal peptide of the invention may be a CRTAC1 signal peptide; an SCGB1A1 signal peptide; an A2M signal peptide; an SFTPA2 signal peptide; or a CLEC3B signal peptide. Non-limiting examples of such signal peptides are described herein. A particularly preferred signal peptide is a CRTAC1 signal peptide. Variants and fragments of such signal peptides as described herein are also encompassed.

In a nucleic acid cassette of the invention, the nucleic acid sequence encoding the exogenous signal peptide is typically 5′ of the nucleic acid sequence encoding the therapeutic protein. The nucleic acid sequence encoding the exogenous signal peptide may be directly 5′ of the nucleic acid sequence encoding the therapeutic protein, or there may be a linker (also referred to interchangeably as a spacer) nucleic acid between the nucleic acid sequence encoding the exogenous signal peptide and the nucleic acid sequence encoding the therapeutic protein. Said linker nucleic acid may be any length provided that the encoded signal peptide is still capable of facilitating expression, secretion and/or membrane insertion of proteins (e.g. therapeutic proteins as described herein) by airway cells as described herein. The linker may be 1 to 60 nucleic acids in length, typically 1 to 30, 1 to 20 or 1 to 10. The linker may encode for 1 to 20 amino acids, typically 1 to 10 or 1 to 5.

The invention also relates to the use of an exogenous signal peptide, such as those described above, to increase the expression, secretion and/or membrane insertion of a therapeutic protein by an airway cell.

### Therapeutic Proteins

A nucleic acid cassette of the invention comprises a nucleic acid encoding for a therapeutic protein. A therapeutic protein is one which has potential utility in the treatment or prevention of a disease or condition, such as those describe herein. Thus, a nucleic acid cassette of the invention comprises a nucleic acid encoding a protein which has a therapeutic effect on a disease or condition to be treated.

A nucleic acid cassette of the invention may comprise a nucleic acid encoding a therapeutic protein which is a functional or wild-type form of a protein which is present in a patient to be treated in a dysfunctional form (whether the dysfunction is inherent or acquired). As used herein, the phrase “inherent dysfunction” refers to a protein which is innately dysfunctional due to genetic factors and the phrase “acquired dysfunction” refers to a protein which is dysfunctional due to environmental or other factors after birth. By way of non-limiting example, CFTR is an example of a protein which is inherently dysfunctional in patients with cystic fibrosis.

Thus, a nucleic acid cassette of the invention may comprise a nucleic acid encoding a therapeutic protein which is a functional or wild-type form of a protein which is present in a patient, but which that has become dysfunctional due to a genetic disease, such as a genetic respiratory disease.

The nucleic acid cassettes of the present invention are useful in the treatment of diseases via their use in expressing therapeutic proteins in airway cells: (i) within the respiratory tract; (ii) for secretion from said cells into the lumen of the respiratory tract; and (iii) for secretion from said cells into the circulatory system.

The therapeutic protein may be selected from: (a) a secreted therapeutic protein, optionally alpha-1-antitrypsin (AAT), Factor VIII, Surfactant Protein B (SFTPB), Factor VII, Factor IX, Factor X, Factor XI, von Willebrand Factor, Granulocyte-Macrophage Colony-Stimulating Factor (GM-CSF), Surfactant Protein C (SP-C), an anti-inflammatory protein (e.g. IL-10 or TGGβ) or monoclonal antibody, an anti-inflammatory decoy and a monoclonal antibody against an infectious agent; or (b) CFTR, CSF2RA, CSF2RB and ATP-binding cassette sub-family member A (ABCA3). Preferred examples of therapeutic proteins include AAT, GM-CSF, FVIII, CFTR, decorin, TRIM72 and ABAC3.

The transgene may encode: (i) a therapeutic protein that is secreted into epithelial lining fluid and/or blood); (ii) a therapeutic protein that is secreted into blood); or (iii) a therapeutic membrane protein). Preferred examples of these classes of transgenes include (i) AAT; (ii) FVIII; and (iii) CFTR.

In some embodiments, the therapeutic protein is not an antibody, particularly not a monoclonal antibody. In such embodiments, the therapeutic protein may be selected from: (a) a secreted therapeutic protein, optionally alpha-1-Antitrypsin (AAT), Factor VIII, Surfactant Protein B (SFTPB), Factor VII, Factor IX, Factor X, Factor XI, von Willebrand Factor, Granulocyte-Macrophage Colony-Stimulating Factor (GM-CSF), Surfactant Protein C (SP-C), an anti-inflammatory protein (e.g. IL-10, TGGβ, or TNF-alpha) and an anti-inflammatory decoy; or (b) CFTR, CSF2RA, CSF2RB and ATP-binding cassette sub-family member A (ABCA3).

The nucleic acid cassettes of the invention are particularly efficient at driving the expression, secretion and/or membrane insertion of proteins (e.g. therapeutic proteins as described herein) by airway cells. This is particularly the case when such cassettes are comprised within F/HN pseudotyped viral vectors of the invention (as described herein), which are efficient at targeting cells in the airway epithelium.

As such, for therapeutic applications the nucleic acid cassettes of the invention (and vectors comprising said cassettes) are typically delivered to cells of the respiratory tract, including the cells of the airway epithelium. In other words, the nucleic acid cassettes of the invention (and vectors comprising said cassettes) are typically delivered to airway cells as described herein. Accordingly, the nucleic acid cassettes of the invention (and vectors comprising said cassettes) are particularly suited for treatment of diseases or disorders of the airways, respiratory tract, or lung. Typically, the nucleic acid cassettes of the invention (and vectors comprising said cassettes) may be used for the treatment of a genetic respiratory disease.

A nucleic acid cassette of the invention (or vector comprising said cassette) may comprise a nucleic acid encoding a polypeptide or protein that is therapeutic for the treatment of such diseases, particularly a disease or disorder of the airways, respiratory tract, or lung. The transgene and therapeutic protein of the invention are not limited, one of ordinary skill in the art will be able to identify therapeutic proteins which may be usefully delivered according to the invention, particularly in the context of genetic diseases, particularly genetic respiratory diseases and diseases or disorders of the airways, respiratory tract, or lung such as those described herein.

Accordingly, a nucleic acid cassette of the invention (or vector comprising said cassette) may comprise a nucleic acid sequence encoding a therapeutic protein selected from: (a) a secreted therapeutic protein, optionally alpha-1-antitrypsin (AAT), Factor VIII, Surfactant Protein B (SFTPB), Factor VII, Factor IX, Factor X, Factor XI, von Willebrand Factor, Granulocyte-Macrophage Colony-Stimulating Factor (GM-CSF), Surfactant Protein C (SP-C), an anti-inflammatory protein (e.g. IL-10, TGGβ, or TNF-alpha) or monoclonal antibody, an anti-inflammatory decoy and a monoclonal antibody against an infectious agent; or (b) CFTR, CSF2RA, CSF2RB and ATP-binding cassette sub-family member A (ABCA3). Other examples of therapeutic proteins that may be encoded by a nucleic acid sequence comprised in a nucleic acid cassette of the invention (or vector comprising said cassette) include genes related to or associated with other surfactant deficiencies. Preferred examples of therapeutic proteins include AAT, GM-CSF, FVIII, CFTR, decorin, TRIM72 and ABAC3.

The therapeutic protein encoded by a nucleic acid cassette of the invention (or vector comprising said cassette) may be an AAT. An example of an AAT therapeutic transgene (SERPINA1) is provided by SEQ ID NO: 22, or by the complementary sequence of SEQ ID NO: 23. SEQ ID NO: 22 is a codon-optimized CpG depleted AAT transgene (SERPINA1) previously designed by the present inventors to enhance translation in human cells. Such optimisation has been shown to enhance gene expression by up to 15-fold. Variants of same sequence (as defined herein) which possess the same technical effect of enhancing translation compared with the unmodified (wild-type) AAT gene sequence are also encompassed by the present invention. The therapeutic protein encoded by said AAT transgene, may be exemplified by the polypeptide of SEQ ID NO: 24. Variants thereof (as described therein) are also included, particularly variants with at least 90% (such as at least 90, 92, 94, 95, 96, 97, 98, 99 or 100% to any one of SEQ ID NO: 22, 23 or 24.

The therapeutic protein encoded by a nucleic acid cassette of the invention (or vector comprising said cassette) may be an FVIII. Examples of a FVIII therapeutic transgene are provided by SEQ ID NOs: 25 and 26, or by the respective complementary sequences of SEQ ID NO: 27 and 28. The polypeptide encoded by the FVIII transgene, may be exemplified by the polypeptide of SEQ ID NO: 29 or 30. Variants thereof (as described therein) are also included, particularly variants with at least 90% (such as at least 90, 92, 94, 95, 96, 97, 98, 99 or 100% to any one of SEQ ID NOs: 25 to 30.

Preferably, the therapeutic protein encoded by a nucleic acid cassette of the invention (or vector comprising said cassette) is a CFTR. An example of a CFTR transgene is provided by SEQ ID NO: 16. The polypeptide encoded by said CFTR transgene, may be exemplified by the polypeptide of SEQ ID NO: 17. Variants thereof (as described therein) are also included, particularly variants with at least 90% (such as at least 90, 92, 94, 95, 96, 97, 98, 99 or 100% to SEQ ID NO: 16 or 17.

The therapeutic protein encoded by a nucleic acid cassette of the invention (or vector comprising said cassette) may be GM-CSF. A GM-CSF transgene may comprise or consist of SEQ ID NO: 18 (human). The polypeptide encoded by the GM-CSF transgene may be exemplified by the polypeptide of SQE ID NO: 19 (human). Variants thereof (as described therein) are also included, particularly variants with at least 90% (such as at least 90, 92, 94, 95, 96, 97, 98, 99 or 100% to any one of SEQ ID NOs: 18 and 19.

The transgene may encode decorin. An example of a DCN transgene is provided by SEQ ID NO: 31. The polypeptide encoded by said DCN transgene, may be exemplified by the polypeptide of SEQ ID NO: 32. Variants thereof (as described therein) are also included, particularly variants with at least 90% (such as at least 90, 92, 94, 95, 96, 97, 98, 99 or 100% to SEQ ID NO: 31 or 32.

The transgene may encode TRIM72. An example of a TRIM72 transgene is provided by SEQ ID NO: 33. The polypeptide encoded by said TRIM72 transgene, may be exemplified by the polypeptide of SEQ ID NO: 34. Variants thereof (as described therein) are also included, particularly variants with at least 90% (such as at least 90, 92, 94, 95, 96, 97, 98, 99 or 100% to SEQ ID NO: 33 or 34.

The transgene may encode ABCA3. An example of a ABACA3 transgene is provided by SEQ ID NO: 35. The polypeptide encoded by said ABACA3 transgene, may be exemplified by the polypeptide of SEQ ID NO: 36. Variants thereof (as described therein) are also included, particularly variants with at least 90% (such as at least 90, 92, 94, 95, 96, 97, 98, 99 or 100% to SEQ ID NO: 35 or 36.

The therapeutic protein encoded by a nucleic acid cassette of the invention (or vector comprising said cassette) may be encoded by any one of SFTPB, SFTPC, Factor V, Factor VII, Factor IX, Factor X and/or Factor XI, von Willebrand Factor, GM-CSF, ABCA3, TRIM72 or DCN, or other known related gene.

When the respiratory tract epithelium is targeted for delivery of the nucleic acid cassettes of the invention (and vectors comprising said cassettes), the therapeutic protein may be AAT, SFTPB, or GM-CSF. The therapeutic protein may be a monoclonal antibody (mAb) against an infectious agent (bacterial, fungal or viral, e.g. the SARS-Co-V2 virus). The therapeutic protein may be anti-TNF alpha. The therapeutic protein may be one implicated in an inflammatory, immune or metabolic condition.

A nucleic acid cassette of the invention (or a vector comprising said cassette) may be delivered to the cells of the respiratory tract to allow production of proteins to be secreted into circulatory system. In such embodiments, the therapeutic protein may be any one of Factor VII, Factor VIII, Factor IX, Factor X, Factor XI and/or von Willebrand's factor. Such a nucleic acid cassette of the invention (or a vector comprising said cassette) may be used in the treatment of diseases, particularly cardiovascular diseases and blood disorders, preferably blood clotting deficiencies such as haemophilia. Again, the therapeutic protein may be an mAb against an infectious agent or a protein implicated in an inflammatory, immune or metabolic condition, such as, lysosomal storage disease.

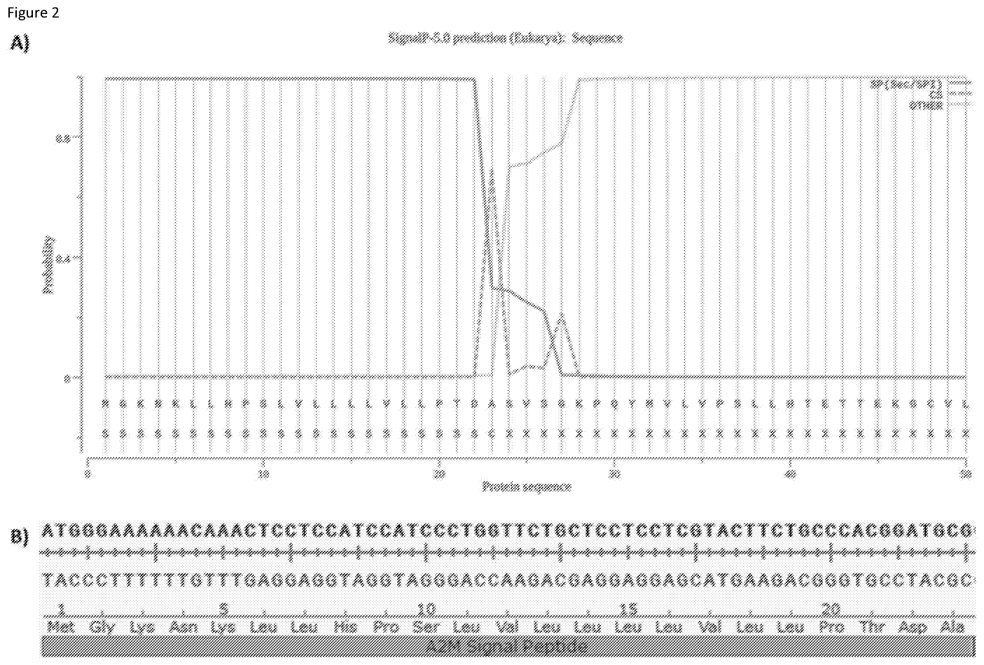

The nucleic acid cassette of the invention (or a vector comprising said cassette) may have no intron positioned between the promoter and the nucleic acid encoding the therapeutic protein. Similarly, when the nucleic acid cassette is comprised in a viral vector, there may be no intron between the promoter and the transgene in the vector genome (pDNA1) plasmid used to make said viral vector. By way of non-limiting example, pGM326 or pGM830 as illustrated in FIGS. 2A and B and the corresponding sequences in UK Application No. 2102832.9, which is herein incorporated by reference in its entirety.

The nucleic acid cassette of the invention (or a vector comprising said cassette) may comprise a hCEF promoter and a CFTR transgene, including those described herein. Optionally said nucleic acid cassette of the invention (or a vector comprising said cassette) may have no intron positioned between the promoter and the transgene.

The nucleic acid cassette of the invention (or a vector comprising said cassette) may comprise a hCEF promoter and an AAT transgene (SERPINA1), including those described herein. Optionally said nucleic acid cassette of the invention (or a vector comprising said cassette) may have no intron positioned between the promoter and the transgene.

The nucleic acid cassette of the invention (or a vector comprising said cassette) may comprise a hCEF or CMV promoter and an FVIII transgene, including those described herein. Optionally said nucleic acid cassette of the invention (or a vector comprising said cassette) may have no intron positioned between the promoter and the transgene.

In some preferred embodiments, the lentiviral (e.g. SIV) vector comprises a hCEF or CMV promoter and an DCN transgene, including those described herein. Optionally said lentiviral (e.g. SIV) vector may have no intron positioned between the promoter and the transgene. Such a lentiviral (e.g. SIV) vector may be produced by the method described herein, using a genome plasmid carrying the DCN transgene and a promoter.

In some preferred embodiments, the lentiviral (e.g. SIV) vector comprises a hCEF or CMV promoter and an TRIM72 transgene, including those described herein. Optionally said lentiviral (e.g. SIV) vector may have no intron positioned between the promoter and the transgene. Such a lentiviral (e.g. SIV) vector may be produced by the method described herein, using a genome plasmid carrying the TRIM72 transgene and a promoter.

In some preferred embodiments, the lentiviral (e.g. SIV) vector comprises a hCEF or CMV promoter and an ABACA3 transgene, including those described herein. Optionally said lentiviral (e.g. SIV) vector may have no intron positioned between the promoter and the transgene. Such a lentiviral (e.g. SIV) vector may be produced by the method described herein, using a genome plasmid carrying the ABACA3 transgene and a promoter.

The nucleic acid cassette of the invention (or a vector comprising said cassette) comprises a nucleic acid encoding a therapeutic protein (said nucleic acid is referred to interchangeably herein as a transgene). The nucleic acid sequence encodes a gene product, e.g., a protein, particularly a therapeutic protein.

For example, the nucleic acid cassette of the invention (or a vector comprising said cassette) comprises a nucleic acid sequence encoding an AAT, GM-CSF, FVIII, CFTR, decorin, TRIM72 or ABAC3 and said nucleic acid sequence comprises (or consists of) a nucleic acid sequence having at least 90% (such as at least 90, 92, 94, 95, 96, 97, 98, 99 or 100%) sequence identity to the AAT, GM-CSF, FVIII, CFTR, decorin, TRIM72 or ABAC3 nucleic acid sequence respectively, examples of which are described herein. In a further embodiment, the nucleic acid sequence encoding AAT, GM-CSF, FVIII, CFTR, decorin, TRIM72 or ABAC3 comprises (or consists of) a nucleic acid sequence having at least 95% (such as at least 95, 96, 97, 98, 99 or 100%) sequence identity to the AAT, GM-CSF, FVIII, CFTR, decorin, TRIM72 or ABAC3 nucleic acid sequence respectively, examples of which are described herein. In one embodiment, the nucleic acid sequence encoding CFTR is provided by SEQ ID NO: 16, the nucleic acid sequence encoding AAT is provided by SEQ ID NO: 22, or by the complementary sequence of SEQ ID NO: 23 and/or the nucleic acid sequence encoding FVIII is provided by SEQ ID NO: 25 or 26, or by the respective complementary sequences of SEQ ID NO: 27 or 28, the nucleic acid sequence encoding GM-CSF is provided by SEQ ID NO: 18, the nucleic acid sequence encoding decorin is provided by SEQ ID NO: 31, the nucleic acid sequence encoding TRIM72 is provided by SEQ ID NO: 33, and/or the nucleic acid sequence encoding ABCA3 is provided by SEQ ID NO: 35, or variants thereof.

The amino acid sequence of the therapeutic protein may be a functional variant having at least 95% (such as at least 95, 96, 97, 98, 99 or 100%) sequence identity to the functional protein. For example, an AAT, FVIII, CFTR, GM-CSF, decorin, TRIM72 and/or ABCA3 polypeptide encoded by the respective AAT, FVIII, CFTR, CSF2, DCN, TRIM72, and/or ABACA3 transgene may comprise (or consist of) an amino acid sequence having at least 95% (such as at least 95, 96, 97, 98, 99 or 100%) sequence identity to the functional AAT, FVIII, CFTR, GM-CSF, decorin, TRIM72 and/or ABCA3 polypeptide sequence respectively.

The transgene encoding for a therapeutic protein may include a nucleic acid sequence encoding for the endogenous signal peptide of the therapeutic protein, or may exclude a nucleic acid sequence encoding for this signal peptide. Typically, the transgene encoding for a therapeutic protein excludes a nucleic acid sequence encoding for the endogenous signal peptide of the therapeutic protein. In such instances, the exogenous signal peptide provided by the invention is typically the sole signal peptide linked with (and hence driving secretion and/or membrane insertion) of the therapeutic protein. Where appropriate, endogenous signal peptides have been identified in the sequence information section herein. All disclosure herein relates to both transgenes and therapeutic proteins including and excluding endogenous signal peptides unless explicitly stated. By way of non-limiting example, sequence identity of variants, and/or lengths of fragments may be based on the sequence with or without a signal peptide.

Any combination of signal peptide and therapeutic protein may be encoded by a nucleic acid cassette of the invention, provided that the signal peptide is effective at enhancing the expression, secretion and/or membrane insertion of said therapeutic protein by airway cells. Typically the signal peptide and therapeutic protein are both independently selected from those described herein.

Thus, the invention relates to nucleic acid cassettes encoding a CRTAC1 signal peptide and a therapeutic protein. In particular, the invention relates to nucleic acid cassettes encoding: a CRTAC1 signal peptide and AAT; a CRTAC1 signal peptide and FVIII; a CRTAC1 signal peptide and SFTPB; a CRTAC1 signal peptide and Factor VII; a CRTAC1 signal peptide and Factor IX; a CRTAC1 signal peptide and Factor X; a CRTAC1 signal peptide and Factor XI; a CRTAC1 signal peptide and von Willebrand Factor; a CRTAC1 signal peptide and GM-CSF; a CRTAC1 signal peptide and SFTPC; a CRTAC1 signal peptide and ABCA3; a CRTAC1 signal peptide and decorin; a CRTAC1 signal peptide and TRIM72; a CRTAC1 signal peptide and an anti-inflammatory protein (e.g. IL-10 or TGGβ) or monoclonal antibody; a CRTAC1 signal peptide and an anti-inflammatory decoy; a CRTAC1 signal peptide and a monoclonal antibody against an infectious agent; a CRTAC1 signal peptide and CFTR; a CRTAC1 signal peptide and CSF2RA; and/or a CRTAC1 signal peptide and CSF2RB. In some embodiments the therapeutic protein is not an antibody.

The invention also relates to nucleic acid cassettes encoding an AAT signal peptide and a therapeutic protein. In particular, the invention relates to nucleic acid cassettes encoding: an AAT signal peptide and FVIII; an AAT signal peptide and SFTPB; an AAT signal peptide and Factor VII; an AAT signal peptide and Factor IX; an AAT signal peptide and Factor X; an AAT signal peptide and Factor XI; an AAT signal peptide and von Willebrand Factor; an AAT signal peptide and GM-CSF; an AAT signal peptide and SFTPC; an AAT signal peptide and ABCA3; an AAT signal peptide and decorin; an AAT signal peptide and TRIM72; an AAT signal peptide and an anti-inflammatory protein (e.g. IL-10 or TGGβ) or monoclonal antibody; an AAT signal peptide and an anti-inflammatory decoy; an AAT signal peptide and a monoclonal antibody against an infectious agent; an AAT signal peptide and CFTR; an AAT signal peptide and CSF2RA; and/or an AAT signal peptide and CSF2RB. In some embodiments the therapeutic protein is not an antibody.

The invention also relates to nucleic acid cassettes encoding: a CLEC3B signal peptide and a therapeutic protein. In particular, the invention relates to nucleic acid cassettes encoding: a CLEC3B signal peptide and AAT; a CLEC3B signal peptide and FVIII; a CLEC3B signal peptide and SFTPB; a CLEC3B signal peptide and Factor VII; a CLEC3B signal peptide and Factor IX; a CLEC3B signal peptide and Factor X; a CLEC3B signal peptide and Factor XI; a CLEC3B signal peptide and von Willebrand Factor; a CLEC3B signal peptide and GM-CSF; a CLEC3B signal peptide and SFTPC; a CLEC3B signal peptide and ABCA3; a CLEC3B signal peptide and decorin; a CLEC3B signal peptide and TRIM72; a CLEC3B signal peptide and an anti-inflammatory protein (e.g. IL-10 or TGGβ) or monoclonal antibody; a CLEC3B signal peptide and an anti-inflammatory decoy; a CLEC3B signal peptide and a monoclonal antibody against an infectious agent; a CLEC3B signal peptide and CFTR; a CLEC3B signal peptide and CSF2RA; and/or a CLEC3B signal peptide and CSF2RB. In some embodiments the therapeutic protein is not an antibody.

The invention also relates to nucleic acid cassettes encoding an SCGB1A1 signal peptide and a therapeutic protein. In particular, the invention relates to nucleic acid cassettes encoding: an SCGB1A1 signal peptide and FVIII; an SCGB1A1 signal peptide and SFTPB; an SCGB1A1 signal peptide and Factor VII; an SCGB1A1 signal peptide and Factor IX; an SCGB1A1 signal peptide and Factor X; an SCGB1A1 signal peptide and Factor XI; an SCGB1A1 signal peptide and von Willebrand Factor; an SCGB1A1 signal peptide and GM-CSF; an SCGB1A1 signal peptide and SFTPC; an SCGB1A1 signal peptide and ABCA3; an SCGB1A1 signal peptide and AAT; an SCGB1A1 signal peptide and decorin; an SCGB1A1 signal peptide and TRIM72; an SCGB1A1 signal peptide and an anti-inflammatory protein (e.g. IL-10 or TGGβ) or monoclonal antibody; an SCGB1A1 signal peptide and an anti-inflammatory decoy; an SCGB1A1 signal peptide and a monoclonal antibody against an infectious agent; an SCGB1A1 signal peptide and CFTR; an SCGB1A1 signal peptide and CSF2RA; and/or an SCGB1A1 signal peptide and CSF2RB. In some embodiments the therapeutic protein is not an antibody.

The invention also relates to nucleic acid cassettes encoding an A2M signal peptide and a therapeutic protein. In particular, the invention relates to nucleic acid cassettes encoding: an A2M signal peptide and FVIII; an A2M signal peptide and SFTPB; an A2M signal peptide and Factor VII; an A2M signal peptide and Factor IX; an A2M signal peptide and Factor X; an A2M signal peptide and Factor XI; an A2M signal peptide and von Willebrand Factor; an A2M signal peptide and GM-CSF; an A2M signal peptide and SFTPC; an A2M signal peptide and ABCA3; an A2M signal peptide and decorin; an A2M signal peptide and TRIM72; an A2M signal peptide and an anti-inflammatory protein (e.g. IL-10 or TGGβ) or monoclonal antibody; an A2M signal peptide and an anti-inflammatory decoy; an A2M signal peptide and a monoclonal antibody against an infectious agent; an A2M signal peptide and CFTR; an A2M signal peptide and CSF2RA; and/or an A2M signal peptide and CSF2RB. In some embodiments the therapeutic protein is not an antibody.

The invention also relates to nucleic acid cassettes encoding an SFTPA2 signal peptide and a therapeutic protein. In particular, the invention relates to nucleic acid cassettes encoding: an SFTPA2 signal peptide and FVIII; an SFTPA2 signal peptide and SFTPB; an SFTPA2 signal peptide and Factor VII; an SFTPA2 signal peptide and Factor IX; an SFTPA2 signal peptide and Factor X; an SFTPA2 signal peptide and Factor XI; an SFTPA2 signal peptide and von Willebrand Factor; an SFTPA2 signal peptide and GM-CSF; an SFTPA2 signal peptide and SFTPC; an SFTPA2 signal peptide and ABCA3; an SFTPA2 signal peptide and decorin; an SFTPA2 signal peptide and TRIM72; an SFTPA2 signal peptide and an anti-inflammatory protein (e.g. IL-10 or TGGβ) or monoclonal antibody; an SFTPA2 signal peptide and an anti-inflammatory decoy; an SFTPA2 signal peptide and a monoclonal antibody against an infectious agent; an SFTPA2 signal peptide and CFTR; an SFTPA2 signal peptide and CSF2RA; and/or an SFTPA2 signal peptide and CSF2RB. In some embodiments the therapeutic protein is not an antibody.

The invention also relates to nucleic acid cassettes encoding: a GM-CSF signal peptide and a therapeutic protein. In particular, the invention relates to nucleic acid cassettes encoding: a GM-CSF signal peptide and AAT; a GM-CSF signal peptide and FVIII; a GM-CSF signal peptide and SFTPB; a GM-CSF signal peptide and Factor VII; a GM-CSF signal peptide and Factor IX; a GM-CSF signal peptide and Factor X; a GM-CSF signal peptide and Factor XI; a GM-CSF signal peptide and von Willebrand Factor; a GM-CSF signal peptide and SFTPC; a GM-CSF signal peptide and ABCA3; a GM-CSF signal peptide and decorin; a GM-CSF signal peptide and TRIM72; a GM-CSF signal peptide and an anti-inflammatory protein (e.g. IL-10 or TGGβ) or monoclonal antibody; a GM-CSF signal peptide and an anti-inflammatory decoy; a GM-CSF signal peptide and a monoclonal antibody against an infectious agent; a GM-CSF signal peptide and CFTR; a GM-CSF signal peptide and CSF2RA; and/or a GM-CSF signal peptide and CSF2RB. In some embodiments the therapeutic protein is not an antibody.

The invention also relates to nucleic acid cassettes encoding an IDS signal peptide and a therapeutic protein. In particular, the invention relates to nucleic acid cassettes encoding: an IDS signal peptide and FVIII; an IDS signal peptide and SFTPB; an IDS signal peptide and Factor VII; an IDS signal peptide and Factor IX; an IDS signal peptide and Factor X; an IDS signal peptide and Factor XI; an IDS signal peptide and von Willebrand Factor; an IDS signal peptide and GM-CSF; an IDS signal peptide and SFTPC; an IDS signal peptide and ABCA3; an IDS signal peptide and decorin; an IDS signal peptide and TRIM72; an IDS signal peptide and an anti-inflammatory protein (e.g. IL-10 or TGGβ) or monoclonal antibody; an IDS signal peptide and an anti-inflammatory decoy; an IDS signal peptide and a monoclonal antibody against an infectious agent; an IDS signal peptide and CFTR; an IDS signal peptide and CSF2RA; and/or an IDS signal peptide and CSF2RB. In some embodiments the therapeutic protein is not an antibody.

The invention also relates to nucleic acid cassettes encoding a synthetic (e.g. Secrecon) signal peptide and a therapeutic protein. In particular, the invention relates to nucleic acid cassettes encoding: a synthetic (e.g. Secrecon) signal peptide and AAT; a synthetic (e.g. Secrecon) signal peptide and FVIII; a synthetic (e.g. Secrecon) signal peptide and SFTPB; a synthetic (e.g. Secrecon) signal peptide and Factor VII; a synthetic (e.g. Secrecon) signal peptide and Factor IX; a synthetic (e.g. Secrecon) signal peptide and Factor X; a synthetic (e.g. Secrecon) signal peptide and Factor XI; a synthetic (e.g. Secrecon) signal peptide and von Willebrand Factor; a synthetic (e.g. Secrecon) signal peptide and GM-CSF; a synthetic (e.g. Secrecon) signal peptide and SFTPC; a synthetic (e.g. Secrecon) signal peptide and ABCA3; a synthetic (e.g. Secrecon) peptide and decorin; a synthetic (e.g. Secrecon) signal peptide and TRIM72; a synthetic (e.g. Secrecon) signal peptide and an anti-inflammatory protein (e.g. IL-10 or TGGβ) or monoclonal antibody; a synthetic (e.g. Secrecon) signal peptide and an anti-inflammatory decoy; a synthetic (e.g. Secrecon) signal peptide and a monoclonal antibody against an infectious agent; a synthetic (e.g. Secrecon) signal peptide and CFTR; a synthetic (e.g. Secrecon) signal peptide and CSF2RA; and/or a synthetic (e.g. Secrecon) signal peptide and CSF2RB. In some embodiments the therapeutic protein is not an antibody.

The invention also relates to nucleic acid cassettes encoding a hybrid signal peptide and a therapeutic protein. In particular, the invention relates to nucleic acid cassettes encoding: a hybrid signal peptide and AAT; a hybrid signal peptide and FVIII; a hybrid signal peptide and SFTPB; a hybrid signal peptide and Factor VII; a hybrid signal peptide and Factor IX; a hybrid signal peptide and Factor X; a hybrid signal peptide and Factor XI; a hybrid signal peptide and von Willebrand Factor; a hybrid signal peptide and GM-CSF; a hybrid signal peptide and SFTPC; a hybrid signal peptide and ABCA3; a hybrid signal peptide and an anti-inflammatory protein (e.g. IL-10 or TGGβ) or monoclonal antibody; a hybrid signal peptide and an anti-inflammatory decoy; a hybrid signal peptide and a monoclonal antibody against an infectious agent; a hybrid signal peptide and CFTR; a hybrid signal peptide and CSF2RA; and/or a hybrid signal peptide and CSF2RB. In some embodiments the therapeutic protein is not an antibody.

By way of non-limiting example, the invention relates to nucleic acid cassettes encoding a hybrid Secrecon/CRTAC1 signal peptide (as defined herein) and a therapeutic protein. In particular, the invention relates to nucleic acid cassettes encoding: a hybrid Secrecon/CRTAC1 signal peptide and AAT; a hybrid Secrecon/CRTAC1 signal peptide and FVIII; a hybrid Secrecon/CRTAC1 signal peptide and SFTPB; a hybrid Secrecon/CRTAC1 signal peptide and Factor VII; a hybrid Secrecon/CRTAC1 signal peptide and Factor IX; a hybrid Secrecon/CRTAC1 signal peptide and Factor X; a hybrid Secrecon/CRTAC1 signal peptide and Factor XI; a hybrid Secrecon/CRTAC1 signal peptide and von Willebrand Factor; a hybrid Secrecon/CRTAC1 signal peptide and GM-CSF; a hybrid Secrecon/CRTAC1 signal peptide and SFTPC; a hybrid Secrecon/CRTAC1 signal peptide and ABCA3; a hybrid Secrecon/CRTAC1 signal peptide and decorin; a hybrid Secrecon/CRTAC1 signal peptide and TRIM72; a hybrid Secrecon/CRTAC1 signal peptide and an anti-inflammatory protein (e.g. IL-10 or TGGβ) or monoclonal antibody; a hybrid Secrecon/CRTAC1 signal peptide and an anti-inflammatory decoy; a hybrid Secrecon/CRTAC1 signal peptide and a monoclonal antibody against an infectious agent; a hybrid Secrecon/CRTAC1 signal peptide and CFTR; a hybrid Secrecon/CRTAC1 signal peptide and CSF2RA; and/or a hybrid Secrecon/CRTAC1 signal peptide and CSF2RB.

By way of a further non-limiting example, the invention relates to nucleic acid cassettes encoding a hybrid Secrecon/AAT signal peptide (as defined herein) and a therapeutic protein. In particular, the invention relates to nucleic acid cassettes encoding: a hybrid Secrecon/AAT signal peptide and AAT; a hybrid Secrecon/AAT signal peptide and FVIII; a hybrid Secrecon/AAT signal peptide and SFTPB; a hybrid Secrecon/AAT signal peptide and Factor VII; a hybrid Secrecon/AAT signal peptide and Factor IX; a hybrid Secrecon/AAT signal peptide and Factor X; a hybrid Secrecon/AAT signal peptide and Factor XI; a hybrid Secrecon/AAT signal peptide and von Willebrand Factor; a hybrid Secrecon/AAT signal peptide and GM-CSF; a hybrid Secrecon/AAT signal peptide and SFTPC; a hybrid Secrecon/AAT signal peptide and ABCA3; a hybrid Secrecon/AAT signal peptide and decorin; a hybrid Secrecon/AAT signal peptide and TRIM72; a hybrid Secrecon/AAT signal peptide and an anti-inflammatory protein (e.g. IL-10 or TGGβ) or monoclonal antibody; a hybrid Secrecon/AAT signal peptide and an anti-inflammatory decoy; a hybrid Secrecon/AAT signal peptide and a monoclonal antibody against an infectious agent; a hybrid Secrecon/AAT signal peptide and CFTR; a hybrid Secrecon/AAT signal peptide and CSF2RA; and/or a hybrid Secrecon/AAT signal peptide and CSF2RB. By way of a further non-limiting example, the invention relates to nucleic acid cassettes encoding a hybrid Secrecon/CLEC3B signal peptide (as defined herein) and a therapeutic protein. In particular, the invention relates to nucleic acid cassettes encoding: a hybrid Secrecon/CLEC3B signal peptide and AAT; a hybrid Secrecon/CLEC3B signal peptide and FVIII; a hybrid Secrecon/CLEC3B signal peptide and SFTPB; a hybrid Secrecon/CLEC3B signal peptide and Factor VII; a hybrid Secrecon/CLEC3B signal peptide and Factor IX; a hybrid Secrecon/CLEC3B signal peptide and Factor X; a hybrid Secrecon/CLEC3B signal peptide and Factor XI; a hybrid Secrecon/CLEC3B signal peptide and von Willebrand Factor; a hybrid Secrecon/CLEC3B signal peptide and GM-CSF; a hybrid Secrecon/CLEC3B signal peptide and SFTPC; a hybrid Secrecon/CLEC3B signal peptide and ABCA3; a hybrid Secrecon/CLEC3B signal peptide and decorin; a hybrid Secrecon/CLEC3B signal peptide and TRIM72; a hybrid Secrecon/CLEC3B signal peptide and an anti-inflammatory protein (e.g. IL-10 or TGGβ) or monoclonal antibody; a hybrid Secrecon/CLEC3B signal peptide and an anti-inflammatory decoy; a hybrid Secrecon/CLEC3B signal peptide and a monoclonal antibody against an infectious agent; a hybrid Secrecon/CLEC3B signal peptide and CFTR; a hybrid Secrecon/CLEC3B signal peptide and CSF2RA; and/or a hybrid Secrecon/CLEC3B signal peptide and CSF2RB.

By way of a further non-limiting example, the invention relates to nucleic acid cassettes encoding a hybrid Secrecon/A2M signal peptide (as defined herein) and a therapeutic protein. In particular, the invention relates to nucleic acid cassettes encoding: a hybrid Secrecon/A2M signal peptide and AAT; a hybrid Secrecon/A2M signal peptide and FVIII; a hybrid Secrecon/A2M signal peptide and SFTPB; a hybrid Secrecon/A2M signal peptide and Factor VII; a hybrid Secrecon/A2M signal peptide and Factor IX; a hybrid Secrecon/A2M signal peptide and Factor X; a hybrid Secrecon/A2M signal peptide and Factor XI; a hybrid Secrecon/A2M signal peptide and von Willebrand Factor; a hybrid Secrecon/A2M signal peptide and GM-CSF; a hybrid Secrecon/A2M signal peptide and SFTPC; a hybrid Secrecon/A2M signal peptide and ABCA3; a hybrid Secrecon/A2M signal peptide and decorin; a hybrid Secrecon/A2M signal peptide and TRIM72; a hybrid Secrecon/A2M signal peptide and an anti-inflammatory protein (e.g. IL-10 or TGGβ) or monoclonal antibody; a hybrid Secrecon/A2M signal peptide and an anti-inflammatory decoy; a hybrid Secrecon/A2M signal peptide and a monoclonal antibody against an infectious agent; a hybrid Secrecon/A2M signal peptide and CFTR; a hybrid Secrecon/A2M signal peptide and CSF2RA; and/or a hybrid Secrecon/A2M signal peptide and CSF2RB.

By way of a further non-limiting example, the invention relates to nucleic acid cassettes encoding a hybrid Secrecon/SCGB1A1 signal peptide (as defined herein) and a therapeutic protein. In particular, the invention relates to nucleic acid cassettes encoding: a hybrid Secrecon/SCGB1A1 signal peptide and AAT; a hybrid Secrecon/SCGB1A1 signal peptide and FVIII; a hybrid Secrecon/SCGB1A1 signal peptide and SFTPB; a hybrid Secrecon/SCGB1A1 signal peptide and Factor VII; a hybrid Secrecon/SCGB1A1 signal peptide and Factor IX; a hybrid Secrecon/SCGB1A1 signal peptide and Factor X; a hybrid Secrecon/SCGB1A1 signal peptide and Factor XI; a hybrid Secrecon/SCGB1A1 signal peptide and von Willebrand Factor; a hybrid Secrecon/SCGB1A1 signal peptide and GM-CSF; a hybrid Secrecon/SCGB1A1 signal peptide and SFTPC; a hybrid Secrecon/SCGB1A1 signal peptide and ABCA3; a hybrid Secrecon/SCGB1A1 signal peptide and decorin; a hybrid Secrecon/SCGB1A1 signal peptide and TRIM72; a hybrid Secrecon/SCGB1A1 signal peptide and an anti-inflammatory protein (e.g. IL-10 or TGGβ) or monoclonal antibody; a hybrid Secrecon/SCGB1A1 signal peptide and an anti-inflammatory decoy; a hybrid Secrecon/SCGB1A1 signal peptide and a monoclonal antibody against an infectious agent; a hybrid Secrecon/SCGB1A1 signal peptide and CFTR; a hybrid Secrecon/SCGB1A1 signal peptide and CSF2RA; and/or a hybrid Secrecon/SCGB1A1 signal peptide and CSF2RB.

By way of a further non-limiting example, the invention relates to nucleic acid cassettes encoding a hybrid Secrecon/SFTPA2 signal peptide (as defined herein) and a therapeutic protein. In particular, the invention relates to nucleic acid cassettes encoding: a hybrid Secrecon/SFTPA2 signal peptide and AAT; a hybrid Secrecon/SFTPA2 signal peptide and FVIII; a hybrid Secrecon/SFTPA2 signal peptide and SFTPB; a hybrid Secrecon/SFTPA2 signal peptide and Factor VII; a hybrid Secrecon/SFTPA2 signal peptide and Factor IX; a hybrid Secrecon/SFTPA2 signal peptide and Factor X; a hybrid Secrecon/SFTPA2 signal peptide and Factor XI; a hybrid Secrecon/SFTPA2 signal peptide and von Willebrand Factor; a hybrid Secrecon/SFTPA2 signal peptide and GM-CSF; a hybrid Secrecon/SFTPA2 signal peptide and SFTPC; a hybrid Secrecon/SFTPA2 signal peptide and ABCA3; a hybrid Secrecon/SFTPA2 signal peptide and decorin; a hybrid Secrecon/SFTPA2 signal peptide and TRIM72; a hybrid Secrecon/SFTPA2 signal peptide and an anti-inflammatory protein (e.g. IL-10 or TGGβ) or monoclonal antibody; a hybrid Secrecon/SFTPA2 signal peptide and an anti-inflammatory decoy; a hybrid Secrecon/SFTPA2 signal peptide and a monoclonal antibody against an infectious agent; a hybrid Secrecon/SFTPA2 signal peptide and CFTR; a hybrid Secrecon/SFTPA2 signal peptide and CSF2RA; and/or a hybrid Secrecon/SFTPA2 signal peptide and CSF2RB.

By way of a further non-limiting example, the invention relates to nucleic acid cassettes encoding a hybrid Secrecon/GM-CSF signal peptide (as defined herein) and a therapeutic protein. In particular, the invention relates to nucleic acid cassettes encoding: a hybrid Secrecon/GM-CSF signal peptide and AAT; a hybrid Secrecon/GM-CSF signal peptide and FVIII; a hybrid Secrecon/GM-CSF signal peptide and SFTPB; a hybrid Secrecon/GM-CSF signal peptide and Factor VII; a hybrid Secrecon/GM-CSF signal peptide and Factor IX; a hybrid Secrecon/GM-CSF signal peptide and Factor X; a hybrid Secrecon/GM-CSF signal peptide and Factor XI; a hybrid Secrecon/GM-CSF signal peptide and von Willebrand Factor; a hybrid Secrecon/GM-CSF signal peptide and GM-CSF; a hybrid Secrecon/GM-CSF signal peptide and SFTPC; a hybrid Secrecon/GM-CSF signal peptide and ABCA3; a hybrid Secrecon/GM-CSF signal peptide and decorin; a hybrid Secrecon/GM-CSF signal peptide and TRIM72; a hybrid Secrecon/GM-CSF signal peptide and an anti-inflammatory protein (e.g. IL-10 or TGGβ) or monoclonal antibody; a hybrid Secrecon/GM-CSF signal peptide and an anti-inflammatory decoy; a hybrid Secrecon/GM-CSF signal peptide and a monoclonal antibody against an infectious agent; a hybrid Secrecon/GM-CSF signal peptide and CFTR; a hybrid Secrecon/GM-CSF signal peptide and CSF2RA; and/or a hybrid Secrecon/GM-CSF signal peptide and CSF2RB.

By way of a further non-limiting example, the invention relates to nucleic acid cassettes encoding a hybrid Secrecon/IDS signal peptide (as defined herein) and a therapeutic protein. In particular, the invention relates to nucleic acid cassettes encoding: a hybrid Secrecon/IDS signal peptide and AAT; a hybrid Secrecon/IDS signal peptide and FVIII; a hybrid Secrecon/IDS signal peptide and SFTPB; a hybrid Secrecon/IDS signal peptide and Factor VII; a hybrid Secrecon/IDS signal peptide and Factor IX; a hybrid Secrecon/IDS signal peptide and Factor X; a hybrid Secrecon/IDS signal peptide and Factor XI; a hybrid Secrecon/IDS signal peptide and von Willebrand Factor; a hybrid Secrecon/IDS signal peptide and GM-CSF; a hybrid Secrecon/IDS signal peptide and SFTPC; a hybrid Secrecon/IDS signal peptide and ABCA3; a hybrid Secrecon/IDS signal peptide and decorin; a hybrid Secrecon/IDS signal peptide and TRIM72; a hybrid Secrecon/IDS signal peptide and an anti-inflammatory protein (e.g. IL-10 or TGGβ) or monoclonal antibody; a hybrid Secrecon/IDS signal peptide and an anti-inflammatory decoy; a hybrid Secrecon/IDS signal peptide and a monoclonal antibody against an infectious agent; a hybrid Secrecon/IDS signal peptide and CFTR; a hybrid Secrecon/IDS signal peptide and CSF2RA; and/or a hybrid Secrecon/IDS signal peptide and CSF2RB.

When a hybrid promoter is used, preferably said hybrid promoter is a hybrid Secrecon/AAT signal peptide as described herein.

Preferred signal peptide/therapeutic protein combinations of the invention may include a CRTAC1 signal peptide and GM-CSF; a SCGB1A1 signal peptide and GM-CSF; a synthetic (e.g. Secrecon) signal peptide and AAT; an A2M signal peptide and GM-CSF; an AAT signal peptide and CRTAC1; and/or an A2M signal peptide and SCGB1A1.

Any signal peptide and therapeutic combination may be used, provided that this combination is effective in increasing the expression, secretion and/or membrane insertion of a therapeutic protein as defined herein. Selection of a signal peptide may depend on specific therapeutic protein and/or the specific airway cell type by which the therapeutic protein is to be expressed/secreted/inserted into the cell membrane.

### Nucleic Acid Cassettes

The present invention provides a nucleic acid cassette comprising (a) a nucleic acid sequence encoding an exogenous signal peptide; and (b) a nucleic acid sequence encoding a therapeutic protein.

The nucleic acid sequence encoding the exogenous signal peptide and or the nucleic acid sequence encoding the therapeutic protein may be referred to as a transgene. Typically a transgene comprises a nucleic acid sequence encoding a gene product, e.g., a protein, particularly a therapeutic protein according to the invention. Thus, the terms “nucleic acid sequence encoding the therapeutic protein” and the term “transgene” may be used interchangeably.

The exogenous signal peptide may increase expression of the therapeutic protein by airway cells. The exogenous signal peptide may increase secretion of the therapeutic protein by airway cells, or insertion of the therapeutic protein into the cell membrane of airway cells. The exogenous signal peptide may increase expression and secretion of the therapeutic protein by airway cells. The exogenous signal peptide may increase expression and membrane insertion of the therapeutic protein by airway cells.

The exogenous signal peptide may increase expression of the therapeutic protein by airway cells. The exogenous signal peptide may increase expression of the therapeutic protein by airway cells compared with expression of the therapeutic protein without the exogenous signal peptide. In such instances, typically the nucleic acid cassette comprising the exogenous signal peptide increases expression of the therapeutic protein by airway cells compared with expression of the therapeutic protein using a corresponding nucleic acid cassette without the exogenous signal peptide. The exogenous signal peptide may increase expression of the therapeutic protein by airway cells compared with expression of the therapeutic protein with its endogenous signal peptide. In such instances, the nucleic acid cassette comprising the exogenous signal peptide may increase expression of the therapeutic protein by airway cells compared with expression of the therapeutic protein using a corresponding nucleic acid cassette with the endogenous signal peptide, and/or compared with expression of the therapeutic protein by the wild-type gene (also encoding the endogenous signal peptide) within the airway cells. By way of non-limiting example, a nucleic acid cassette of the invention comprising an exogenous signal peptide of the invention and an AAT transgene (SERPINA1) may increase AAT expression compared with a corresponding nucleic acid cassette which lacks the exogenous signal peptide. By way of a further non-limiting example, a nucleic acid cassette of the invention comprising an exogenous signal peptide of the invention and an AAT transgene (SERPINA1) may increase AAT expression compared with a corresponding nucleic acid cassette which and comprises the endogenous AAT signal peptide.

The increase in expression of the therapeutic protein by an exogenous signal peptide of the invention may be as defined herein. In particular, the increase in expression of the therapeutic protein by an exogenous signal peptide of the invention is an increase of at least about 50%, at least about 60%, at least about 70%, at least about 80% or more. Preferably, the increase in expression of the therapeutic protein by an exogenous signal peptide of the invention is an increase of at least about 50%.

The exogenous signal peptide may increase secretion of the therapeutic protein from airway cells. The exogenous signal peptide may increase secretion of the therapeutic protein from airway cells compared with secretion of the therapeutic protein without the exogenous signal peptide. In such instances, typically the nucleic acid cassette comprising the exogenous signal peptide increases secretion of the therapeutic protein from airway cells compared with secretion of the therapeutic protein using a corresponding nucleic acid cassette without the exogenous signal peptide. The exogenous signal peptide may increase secretion of the therapeutic protein from airway cells compared with secretion of the therapeutic protein with its endogenous signal peptide. In such instances, the nucleic acid cassette comprising the exogenous signal peptide may increase secretion of the therapeutic protein from airway cells compared with secretion of the therapeutic protein using a corresponding nucleic acid cassette with the endogenous signal peptide, and/or compared with secretion of the therapeutic protein by the wild-type gene (also encoding the endogenous signal peptide) within the airway cells. By way of non-limiting example, a nucleic acid cassette of the invention comprising an exogenous signal peptide of the invention and an AAT transgene (SERPINA1) may increase AAT secretion compared with a corresponding nucleic acid cassette which lacks the exogenous signal peptide. By way of a further non-limiting example, a nucleic acid cassette of the invention comprising an exogenous signal peptide of the invention and an AAT transgene (SERPINA1) may increase AAT secretion compared with a corresponding nucleic acid cassette which and comprises the endogenous AAT signal peptide.

The increase in secretion of the therapeutic protein by an exogenous signal peptide of the invention may be as defined herein. In particular, the increase in secretion of the therapeutic protein by an exogenous signal peptide of the invention is an increase of at least about 50%, at least about 60%, at least about 70%, at least about 80% or more. Preferably, the increase in secretion of the therapeutic protein by an exogenous signal peptide of the invention is an increase of at least about 50%.

The exogenous signal peptide may increase insertion of the therapeutic protein into the cell membrane of airway cells. The exogenous signal peptide may increase insertion of the therapeutic protein into the cell membrane of airway cells compared with insertion of the therapeutic protein into the cell membrane of airway cells without the exogenous signal peptide. In such instances, typically the nucleic acid cassette comprising the exogenous signal peptide increase insertion of the therapeutic protein into the cell membrane of airway cells compared with insertion of the therapeutic protein into the cell membrane of airway cells using a corresponding nucleic acid cassette without the exogenous signal peptide. The exogenous signal peptide may increase insertion of the therapeutic protein into the cell membrane of airway cells compared with insertion of the therapeutic protein into the cell membrane of airway cells with its endogenous signal peptide. In such instances, the nucleic acid cassette comprising the exogenous signal peptide may increase insertion of the therapeutic protein into the cell membrane of airway cells compared with insertion of the therapeutic protein into the cell membrane of airway cells using a corresponding nucleic acid cassette with the endogenous signal peptide, and/or compared with insertion of the therapeutic protein into the cell membrane of airway cells by the wild-type gene (also encoding the endogenous signal peptide) within the airway cells. The increase in insertion of the therapeutic protein into the cell membrane of airway cells by an exogenous signal peptide of the invention may be as defined herein. By way of non-limiting example, a nucleic acid cassette of the invention comprising an exogenous signal peptide of the invention and a CFTR transgene may result in an increase in CFTR insertion into the cell membrane of airway cells compared with a corresponding nucleic acid cassette which lacks the exogenous signal peptide. By way of a further non-limiting example, a nucleic acid cassette of the invention comprising an exogenous signal peptide of the invention and an CFTR transgene may result in an increase in CFTR insertion into the cell membrane of airway cells compared with a corresponding nucleic acid cassette which and comprises the endogenous CFTR signal peptide.

In particular, the increase in insertion of the therapeutic protein into the cell membrane of airway cells using an exogenous signal peptide of the invention is an increase of at least about 50%, at least about 60%, at least about 70%, at least about 80% or more. Preferably, the increase insertion of the therapeutic protein into the cell membrane of airway cells using an exogenous signal peptide of the invention is an increase of at least about 50%.

A nucleic acid cassette or vector of the invention enables long-term transgene expression, resulting in long-term expression of a therapeutic protein. As described herein, the phrases “long-term expression”, “sustained expression”, “long-lasting expression” and “persistent expression” are used interchangeably. Long-term expression according to the present invention means expression of a therapeutic protein, preferably at therapeutic levels, for at least 45 days, at least 60 days, at least 90 days, at least 120 days, at least 180 days, at least 250 days, at least 360 days, at least 450 days, at least 730 days or more. Preferably long-term expression means expression for at least 90 days, at least 120 days, at least 180 days, at least 250 days, at least 360 days, at least 450 days, at least 720 days or more, more preferably at least 360 days, at least 450 days, at least 720 days or more. The long-term expression is typically accompanied by long-term secretion or long-term membrane insertion of the therapeutic protein, depending on whether the therapeutic protein is a secreted protein (e.g. AAT or FVIII) or a membrane protein (e.g. CFTR).

Long-term secretion according to the present invention means secretion of a therapeutic protein, preferably at therapeutic levels, for at least 45 days, at least 60 days, at least 90 days, at least 120 days, at least 180 days, at least 250 days, at least 360 days, at least 450 days, at least 730 days or more. Preferably long-term secretion means secretion for at least 90 days, at least 120 days, at least 180 days, at least 250 days, at least 360 days, at least 450 days, at least 720 days or more, more preferably at least 360 days, at least 450 days, at least 720 days or more.

Long-term membrane insertion according to the present invention means that a therapeutic protein is inserted into and present in the cell membrane, preferably at therapeutic levels, for at least 45 days, at least 60 days, at least 90 days, at least 120 days, at least 180 days, at least 250 days, at least 360 days, at least 450 days, at least 730 days or more. Preferably long-term expression means membrane insertion for at least 90 days, at least 120 days, at least 180 days, at least 250 days, at least 360 days, at least 450 days, at least 720 days or more, more preferably at least 360 days, at least 450 days, at least 720 days or more.

In particular, a nucleic acid cassette or vector of the invention may drive (increased) long-lasting expression and secretion/membrane insertion of a therapeutic protein in an airway cell in vivo in a patient. Preferably, a nucleic acid cassette or vector of the invention drives expression and secretion/membrane insertion of a therapeutic protein in an airway cell for at least 45 days, more preferably at least 90 days.

The nucleic acid of the nucleic acid cassette may be as defined herein. The nucleic acid cassette comprise DNA or RNA. Preferably the nucleic acid cassette is DNA.

A nucleic acid cassette of the invention may optionally be codon optimised for expression in a particular cell type, for example, eukaryotic cells (e.g. mammalian cells, yeast cells, insect cells or plants cells) or prokaryotic cells (e.g. E. coli). The term “codon optimised” refers to the replacement of at least one codon within a base polynucleotide sequence with a codon that is preferentially used by the host organism in which the polynucleotide is to be expressed. Typically, the most frequently used codons in the host organism are used in the codon-optimised polynucleotide sequence. Methods of codon optimisation are well known in the art.

It will be understood by a skilled person that numerous different polynucleotides can encode the same polypeptide as a result of the degeneracy of the genetic code. It is also understood that skilled persons may, using routine techniques, make nucleotide substitutions that do not affect the polypeptide sequence encoded by the nucleic acid molecules to reflect the codon usage of any particular host organism in which the polypeptides are to be expressed. Therefore, unless otherwise specified, a nucleic acid cassette that encodes the signal peptide and therapeutic protein of the invention includes all polynucleotide sequences that are degenerate versions of each other and that encode the same amino acid sequence.

A nucleic acid cassette of the invention typically comprises a promoter operably linked to the nucleic acid sequence encoding the signal peptide and/or the nucleic acid sequence encoding the therapeutic protein. By operably linked, it is meant that the promoter is configured to express the nucleic acid sequence encoding the signal peptide and/or the nucleic acid sequence encoding the therapeutic protein. The nucleic acid sequence encoding the signal peptide and/or the nucleic acid sequence encoding the therapeutic protein may also be linked to a suitable terminator sequence. Suitable promoter and terminator sequences are well known in the art.

By way of non-limiting example, a preferred promoter is a hybrid human CMV enhancer/EF1a (hCEF) promoter. This hCEF promoter may lack the intron corresponding to nucleotides 570-709 and the exon corresponding to nucleotides 728-733 of the hCEF promoter. An example of an hCEF promoter sequence of the invention is provided by SEQ ID NO: 13. The promoter may be a CMV promoter. An example of a CMV promoter sequence is provided by SEQ ID NO: 14. The promoter may be a human elongation factor 1a (EF1a) promoter. An example of a EF1a promoter is provided by SEQ ID NO: 15. Other promoters are known in the art and their suitability for the nucleic acid cassettes and vectors of the invention determined using routine techniques known in the art. Non-limiting examples of other promoters include UbC and UCOE. As described herein, the promoter may be modified to further regulate expression of the transgene of the invention.

The promoter included in the nucleic acid cassettes and vectors of the invention may be specifically selected and/or modified to further refine regulation of expression of the therapeutic gene. Again, suitable promoters and standard techniques for their modification are known in the art. As a non-limiting example, a number of suitable (CpG-free) promoters suitable for use in the present invention are described in Pringle et al. (J. Mol. Med. Berl. 2012, 90(12): 1487-96), which is herein incorporated by reference in its entirety. Preferably, the nucleic acid cassettes and vectors of the invention comprise a hCEF promoter having low or no CpG dinucleotide content. The hCEF promoter may have all CG dinucleotides replaced with any one of AG, TG or GT. Thus, the hCEF promoter may be CpG-free. A preferred example of a CpG-free hCEF promoter sequence of the invention is provided by SEQ ID NO: 13. The absence of CpG dinucleotides further improves the performance of some nucleic acid cassettes and vectors of the invention, particularly lentiviral (e.g. SIV) vectors of the invention and in particular in situations where it is not desired to induce an immune response against an expressed antigen or an inflammatory response against the delivered expression construct. The elimination of CpG dinucleotides reduces the occurrence of flu-like symptoms and inflammation which may result from administration of constructs, particularly when administered to the airways.

The nucleic acid cassettes and vectors of the invention may be modified to allow shut down of gene expression. Standard techniques for modifying the vector in this way are known in the art. As a non-limiting example, Tet-responsive promoters are widely used.

The choice of promoter will depend on where the ultimate expression of the polynucleotide will take place. In general, constitutive promoters are preferred, but inducible promoters may likewise be used.

The nucleic acid cassettes and vectors of the invention may include at least one part of a vector, in particular, regulatory elements. By way of non-limiting example, the promoter (e.g. the hCEFI promoter) within a nucleic acid cassette of the invention may be used to express more than one polypeptide, including one or more therapeutic protein. Thus, the nucleic acid cassette may comprise a nucleic acid sequence which, when transcribed, gives rise to multiple polypeptides, for instance a transcript may contain multiple open reading frames (ORFs) and also one or more Internal Ribosome Entry Sites (IRES) to allow translation of ORFs after the first ORF. A transcript may be polycistronic, i.e. it may be translated to give a polypeptide which is subsequently cleaved to give a plurality of polypeptides. Alternatively, a nucleic acid cassette of the invention may comprise multiple promoters and hence give rise to a plurality of transcripts and hence a plurality of polypeptides, including a plurality of therapeutic proteins. Nucleic acid cassettes may, for instance, express one, two, three, four or more polypeptides via a promoter (e.g. hCEFI) or promoters.

A nucleic acid cassette may comprise one or more translation initiation sequence (TIS). Translation initiation plays an important role in mRNA translation, canonically a methionyl tRNA unique for initiation (Met-tRNAi) identifies the AUG start codon and triggers the downstream translation process. Non-canonical start codons (e.g. CUG for valyl-tRNA)/TIS may also be used.

The nucleic acid cassettes of the present invention may comprise at least one termination signal. A “termination signal” or “terminator” is comprised of the DNA sequences involved in specific termination of an RNA transcript by an RNA polymerase. Thus, a termination signal that ends the production of an RNA transcript is contemplated according to the present invention. A terminator may be necessary in vivo to achieve desirable message levels. In eukaryotic systems, a terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, when the nucleic acid cassette is for expression in eukaryotes, a terminator typically comprises a signal for the cleavage of the RNA, and it is preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

The invention also provides gene therapy vectors comprising a nucleic acid cassette of the invention. Any and all disclosure herein in relation to nucleic acid cassettes of the invention applies equally and without reservation to gene therapy vectors of the invention.

The nucleic acid cassettes and vectors of the invention are capable of expressing the signal peptide and therapeutic protein in a given host cell. Any appropriate host cell may be used, such as mammalian, bacterial, insect, yeast, and/or plant host cells. In addition, cell-free expression systems may be used. Such expression systems and host cells are standard in the art.

Typically the nucleic acid cassettes and vectors of the invention are capable of expressing the signal peptide and therapeutic protein in airway cells. The nucleic acid cassettes and vectors of the invention may be capable of expressing the signal peptide and therapeutic protein in one or more type of airway cell. The nucleic acid cassettes and vectors of the invention may be capable of expressing the signal peptide and therapeutic protein in lung cells. The nucleic acid cassettes and vectors of the invention may be capable of expressing the signal peptide and therapeutic protein in one or more airway cell type selected from epithelial cells, basal cells, submucosal gland duct cells, club cells, neuroendocrine cells, bronchioalveolar stem cells, submucosal acinar cells, ionocytes, type I pneumocytes and/or type II pneumocytes. The nucleic acid cassettes and vectors of the invention are typically capable of expressing the signal peptide and therapeutic protein in one or more long-lived airway epithelial cells or cell types, such as basal cells and submucosal gland duct cells in the upper airways, club cells and neuroendocrine cells in the bronchiolar airways, bronchioalveolar stem cells in the terminal bronchioles and type II pneumocytes in the alveoli.

The nucleic acid cassettes of the invention may be made using any suitable process known in the art. Thus, the nucleic acid cassettes may be made using chemical synthesis techniques. Alternatively, the nucleic acid cassettes of the invention may be made using molecular biology techniques.

### Non-Viral Vectors

The present invention also provides a vector: (a) comprising a nucleic acid cassette of the invention; and/or (b) encoding a signal peptide and therapeutic protein of the invention. The vector(s) may be present in the form of a therapeutic composition or formulation. The vector may be a non-viral vector.

The non-viral vector(s) may be a DNA vector, such as a DNA plasmid. The vector(s) may be an RNA vector, such as a mRNA vector or a self-amplifying RNA vector. The DNA and/or RNA vector(s) of the invention may be capable of expression in eukaryotic and/or prokaryotic cells.

Typically, the DNA and/or RNA vector(s) are capable of expression in a cell of a subject, for example, a cell of a mammalian or avian subject to be immunised.

Typically the nucleic acid cassettes and vectors of the invention are capable of expressing the signal peptide and therapeutic protein in airway cells (as described herein).

A non-viral vector of the present invention may be a phage vector, such as an AAV/phage hybrid vector as described in Hajitou et al., Cell 2006; 125(2) pp. 385-398; herein incorporated by reference.

Vector(s) of the present invention (e.g. non-viral DNA or RNA vectors) may be designed in silico, and then synthesised by conventional polynucleotide synthesis techniques.

Non-viral plasmids cannot replicate in the subject to be treated, as they lack the viral genetic material which hijacks the body's normal production machinery. However they are capable of replicating in appropriate host cells, such as yeasts or bacteria including E. coli, and particularly airway cells as defined herein.

The term “plasmid” as used herein refers to a construction comprised of genetic material designed to direct transformation of a targeted cell. The plasmid contains a plasmid backbone. A “plasmid backbone” as used herein contains multiple genetic elements positionally and sequentially oriented with other necessary genetic elements such that the nucleic acid in the nucleic acid cassette can be transcribed and when necessary translated in the transfected cells.

The plasmid backbone can contain one or more unique restriction sites within the backbone. The plasmid may be capable of autonomous replication in a defined host or organism such that the cloned sequence is reproduced. The plasmid can confer some well-defined phenotype on the host organism which is either selectable or readily detected. The plasmid or plasmid backbone may have a linear or circular configuration. The components of a plasmid can contain, but is not limited to, a DNA molecule incorporating: (1) the plasmid backbone; (2) a sequence encoding a signal peptide; (3) a sequence encoding a therapeutic protein; and (4) regulatory elements for transcription, translation, RNA stability and replication The purpose of the plasmid in human gene therapy for the efficient delivery of nucleic acid sequences to, and expression of therapeutic proteins in, a cell or tissue. In particular, the purpose of the plasmid is to achieve high copy number, avoid potential causes of plasmid instability and provide a means for plasmid selection. As for expression, the nucleic acid cassette contains the necessary elements for expression of the nucleic acid within the cassette. Expression includes the efficient transcription of an inserted gene, nucleic acid sequence, or nucleic acid cassette with the plasmid.

A DNA plasmid may be CpG-free, or be optimised to reduce CpG dinucleotides as described herein. A DNA plasmid of the invention may be codon-optimised as described herein.

Methods of preparing plasmid DNA are well known in the art. Typically, they are capable of autonomous replication in an appropriate host or producer cell.

Host cells containing (e.g. transformed, transfected, or electroporated with) the plasmid may be prokaryotic or eukaryotic in nature, either stably or transiently transformed, transfected, or electroporated with the plasmid. Suitable host cells include bacterial, yeast, fungal, invertebrate, and mammalian cells. Preferably the host cell is bacterial; more preferably E. coli.

Host cells can then be used in methods for the large scale production of the plasmid. The cells are grown in a suitable culture medium under favourable conditions, and the desired plasmid isolated from the cells, or from the medium in which the cells are grown, by any purification technique well known to those skilled in the art; e.g. see Sambrook et al, supra.

Any appropriate delivery means can be used to deliver a non-viral vector (e.g. plasmid) of the invention to a target cell or patient. Suitable delivery means are known in the art and within the routine skill of one of ordinary skill in the art. Non-limiting examples include the use of cationic lipids, polymers (e.g. polyethyleneimine and poly-L-lysine) and electroporation.

Preferably cationic lipids may be used to deliver non-viral (e.g. plasmid) vectors of the invention to target cells or to a patient. Non-limiting examples of cationic lipids suitable for use according to the invention are GL67A and lipofectamine.

The cationic lipid mixture GL67A is a mixture of three components—GL67 (Cholest-5-en-3-ol (3β)-,3-[(3-aminopropyl)[4-[(3-aminopropyl)amino]butyl]carbamate], (CAS Number: 179075-30-0)), DOPE (1,2-dioleoyl-sn-glycero-3-phosphoethanolamine) and DMPE-PEG5000 (1,2-Dimyristoyl-sn-Glycero-3-Phosphoethanolamine-N-[methoxy (Polyethylene glycol)5000]). These components are formulated at a 1:2:0.05 molar ratio to form GL67A. The composition of GL67A and methods for its production are disclosed in WO2013/061091, as are methods for preparing mixtures of GL67A with exemplary non-viral vectors. The contents of WO2013/061091 are herein incorporated by reference in their entirety.

Lipofectamine consists of a 3:1 mixture of DOSPA (2,3-dioleoyloxy-N-[2(sperminecarboxamido)ethyl]-N,N-dimethyl-1-propaniminium trifluoroacetate) and DOPE.

### Viral Vectors

The present invention also provides a vector: (a) comprising a nucleic acid cassette of the invention; and/or (b) encoding a signal peptide and therapeutic protein of the invention. The vector(s) may be present in the form of a therapeutic composition or formulation. The vector may be a viral vector.

Any appropriate viral vector may be used to deliver a nucleic acid cassette of the invention. By way of non-limiting example, a viral vector of the invention may be a lentiviral vector, an adeno-associated virus (AAV) vector, an adenoviral vector, a poxvirus vector and a sendai virus vector.

Non-limiting examples of adenoviral vectors include human serotypes such as AdHu5, simian serotypes such as ChAd63, ChAdOX1 or ChAdOX2, and other forms. Non-limiting examples of poxvirus vectors include a modified vaccinia Ankara (MVA)). ChAdOX1 and ChAdOX2 are disclosed in WO2012/172277 (herein incorporated by reference in its entirety). ChAdOX2 is a BAC-derived and E4 modified AdC68-based viral vector.

Viral vectors are usually non-replicating or replication impaired vectors, which means that the viral vector cannot replicate to any significant extent in normal cells (e.g. normal human cells), as measured by conventional means—e.g. via measuring DNA synthesis and/or viral titre. Non-replicating or replication impaired vectors may have become so naturally (i.e. they have been isolated as such from nature) or artificially (e.g. by breeding in vitro or by genetic manipulation). There will generally be at least one cell-type in which the replication-impaired viral vector can be grown—for example, modified vaccinia Ankara (MVA) can be grown in CEF cells.

Typically, the viral vector is incapable of causing a significant infection in an animal subject, typically in a mammalian subject such as a human or other primate.

Viral vector(s) of the present invention may be designed in silico, and then synthesised by conventional polynucleotide synthesis techniques.

Preferably the invention relates to retroviral vectors, particularly lentiviral vectors. The term “lentivirus” refers to a family of retroviruses. Retroviral/lentiviral vectors of the invention, can integrate into the genome of transduced cells and lead to long-lasting expression. Examples of retroviruses suitable for use in the present invention include gammaretroviruses such as murine leukaemia virus (MLV) and feline leukaemia virus (FLV). Examples of lentiviruses suitable for use in the present invention include Simian immunodeficiency virus (SIV), Human immunodeficiency virus (HIV), Feline immunodeficiency virus (FIV), Equine infectious anaemia virus (EIAV), and Visna/maedi virus. A particularly preferred lentiviral vector is an SIV vector (including all strains and subtypes), such as a SIV-AGM (originally isolated from African green monkeys, Cercopithecus aethiops).

The retroviral/lentiviral (e.g. SIV) vectors of the present invention are typically pseudotyped with hemagglutinin-neuraminidase (HN) and fusion (F) proteins from a respiratory paramyxovirus, or with G glycoprotein from Vesicular Stomatitis Virus (G-VSV). Preferably the lentiviral (e.g. SIV) vectors of the present invention are pseudotyped with HN and F from a respiratory paramyxovirus. Particularly preferably the respiratory paramyxovirus is a Sendai virus (murine parainfluenza virus type 1).

A retroviral/lentiviral (e.g. SIV) vector for use according to the invention may be integrase-competent (IC). Alternatively, the lentiviral (e.g. SIV) vector may be integrase-deficient (ID).

Viral vectors of the invention, particularly retroviral/lentiviral (e.g. SIV) vectors as described herein may transduce one or more cells types as described herein to achieve long term transgene expression.

The viral vectors of the invention, particularly the retroviral/lentiviral (e.g. SIV) vectors of the present invention enable high levels of transgene expression. Together with the increased levels of expression/secretion/membrane insertion of a therapeutic protein resulting from the use of an exogenous signal peptide of the invention, these viral vectors typically result in high levels (therapeutic levels) of expression of a therapeutic protein.

The nucleic acid sequence encoding a therapeutic protein to be included in a viral vector of the invention, particularly a retroviral/lentiviral (e.g. SIV) vector of the invention may be modified to facilitate expression. For example, the transgene sequence may be in CpG-depleted (or CpG-fee) and/or codon-optimised form to facilitate gene expression. Standard techniques for modifying the transgene sequence in this way are known in the art.

The viral vectors of the invention, particularly the retroviral/lentiviral (e.g. SIV) vectors of the invention exhibit enhanced expression of the therapeutic protein. Accordingly, the viral vectors of the invention, particularly the retroviral/lentiviral (e.g. SIV) vectors of the invention are capable of producing long-lasting, repeatable, high-level expression, particularly in airway cells, without inducing an undue immune response.

The viral vectors of the invention, particularly the retroviral/lentiviral (e.g. SIV) vectors of the present invention enable long-term transgene expression, resulting in long-term expression (and secretion or membrane insertion) of a therapeutic protein by airway cells as described herein. Long-term expression according to the present invention means expression of a therapeutic gene and/or protein, preferably at therapeutic levels, for at least 45 days, at least 60 days, at least 90 days, at least 120 days, at least 180 days, at least 250 days, at least 360 days, at least 450 days, at least 730 days or more. Preferably long-term expression means expression for at least 90 days, at least 120 days, at least 180 days, at least 250 days, at least 360 days, at least 450 days, at least 720 days or more, more preferably at least 360 days, at least 450 days, at least 720 days or more.

Preferably, the invention relates to the use of F/HN lentiviral vectors comprising a nucleic acid cassette of the invention, particularly SIV F/HN vectors.

The nucleic acid cassette comprised in a viral vector of the invention, particularly a retroviral/lentiviral (e.g. SIV) vector of the invention, may have no intron positioned between the promoter and the nucleic acid encoding the signal peptide and/or the nucleic acid encoding the therapeutic protein. Similarly, there may be no intron between the promoter and the nucleic acid encoding the signal peptide and/or the nucleic acid encoding the therapeutic protein in the vector genome (pDNA1) plasmid (for example, pGM326 or pGM830 as illustrated in FIGS. 2A and B and the corresponding sequences in UK Application No. 2102832.9, which is herein incorporated by reference in its entirety).

The viral vectors of the invention may be made using any suitable process known in the art. In particular, retroviral/lentiviral (e.g. SIV) vectors of the invention may be made using the methods disclosed in UK Application No. 2102832.9, which is herein incorporated by reference in its entirety).

The viral vectors of the invention, particularly the retroviral/lentiviral (e.g. SIV) vectors of the invention may comprise a central polypurine tract (cPPT) and/or the Woodchuck hepatitis virus posttranscriptional regulatory elements (WPRE). An exemplary WPRE sequence is provided by SEQ ID NO: 37.

### Therapeutic Indications

The nucleic acid cassettes and vectors of the present invention enable higher and sustained expression/secretion/membrane insertion of a therapeutic protein through efficient expression/secretion/membrane insertion of the therapeutic protein. This may be further increased by the nucleic acid cassette or vector facilitating efficient transgene expression. The nucleic acid cassettes and vectors of the invention, and particularly the F/HN-pseudotyped retroviral/lentiviral (e.g. SIV) vectors of the invention are capable of: (i) airway transduction without disruption of epithelial integrity; (ii) persistent gene expression; (iii) lack of chronic toxicity; and/or (iv) efficient repeat administration. Long term/persistent stable gene expression, preferably at a therapeutically-effective level, may be achieved using repeat doses of a nucleic acid cassette or vector of the present invention. Alternatively, a single dose may be used to achieve the desired long-term expression.

Thus, advantageously, the nucleic acid cassettes and vectors of the present invention, and particularly the retroviral/lentiviral (e.g. SIV) vectors of the present invention can be used in gene therapy. Accordingly, the present invention provides a nucleic acid cassette or gene therapy vector as defined herein for use in a method of treating or preventing a disease. The disease to be treated may be chronic or acute.

The nucleic acid cassettes and vectors (viral and non-viral) of the invention, particularly the retroviral/lentiviral (e.g. SIV) vectors of the present invention may be used to deliver any transgene useful in gene therapy. Typically, the nucleic acid cassettes and vectors (viral and non-viral) of the invention, particularly the retroviral/lentiviral (e.g. SIV) vectors of the present invention are for use in gene therapy for the treatment of a disease or disorder of the airways, respiratory tract, or lung.

By way of example, efficient airway cell uptake properties of the nucleic acid cassettes and vectors of the present invention, and particularly the retroviral/lentiviral (e.g. SIV) vectors of the invention make them highly suitable for treating respiratory or respiratory tract diseases, particularly genetic respiratory diseases.

The nucleic acid cassettes and vectors of the present invention, and particularly the retroviral/lentiviral (e.g. SIV) vectors of the invention can also be used in methods of gene therapy to promote secretion of therapeutic proteins. By way of further example, the invention provides secretion of therapeutic proteins into the lumen of the respiratory tract or the circulatory system. Thus, administration of a nucleic acid cassettes and vectors of the present invention, and particularly a retroviral/lentiviral (e.g. SIV) vector of the invention and its uptake by airway cells may enable the use of the lungs (or nose or airways) as a “factory” to produce a therapeutic protein that is then secreted and enters the general circulation at therapeutic levels, where it can travel to cells/tissues of interest to elicit a therapeutic effect. In contrast to intracellular or membrane proteins, the production of such secreted proteins does not rely on specific disease target cells being transduced, which is a significant advantage and achieves high levels of protein expression. Thus, other diseases which are not respiratory tract diseases, such as cardiovascular diseases, particularly genetic cardiovascular diseases or blood disorders, particularly blood clotting deficiencies, can also be treated by the nucleic acid cassettes and vectors of the present invention, and particularly the retroviral/lentiviral (e.g. SIV) vectors of the present invention.

Nucleic acid cassettes and vectors of the present invention, and particularly the retroviral/lentiviral (e.g. SIV) vectors of the invention can effectively treat a disease by providing a transgene for the correction of the disease. For example, inserting a functional copy of the CFTR gene to ameliorate or prevent lung disease in CF patients, independent of the underlying mutation. Accordingly, nucleic acid cassettes and vectors of the present invention, and particularly retroviral/lentiviral (e.g. SIV) vectors of the invention may be used to treat cystic fibrosis (CF), typically by gene therapy with a CFTR transgene as described herein.

As another example, nucleic acid cassettes and vectors of the present invention, and particularly retroviral/lentiviral (e.g. SIV) vectors of the invention may be used to treat alpha-1-antitrypsin (AAT) deficiency, typically by gene therapy with a AAT transgene (SERPINA1) as described herein. AAT is a secreted anti-protease that is produced mainly in the liver and then trafficked to the lung, with smaller amounts also being produced in the lung itself. The main function of AAT is to bind and neutralise/inhibit neutrophil elastase. Gene therapy with AAT according to the present invention is relevant to AAT deficient patient, as well as in other lung diseases such as CF or chronic obstructive pulmonary disease (COPD), and offers the opportunity to overcome some of the problems encountered by conventional enzyme replacement therapy (in which AAT isolated from human blood and administered intravenously every week), providing stable, long-lasting expression in the target tissue (lung/nasal epithelium), ease of administration and unlimited availability.

Transduction with a nucleic acid cassettes and vectors of the present invention, and particularly a retroviral/lentiviral (e.g. SIV) vector of the invention may lead to secretion of the recombinant protein into the lumen of the lung as well as into the circulation. One benefit of this is that the therapeutic protein reaches the interstitium. AAT gene therapy may therefore also be beneficial in other disease indications, non-limiting examples of which include type 1 and type 2 diabetes, acute myocardial infarction, ischemic heart disease, rheumatoid arthritis, inflammatory bowel disease, transplant rejection, graft versus host (GvH) disease, multiple sclerosis, liver disease, cirrhosis, vasculitides and infections, such as bacterial and/or viral infections.

AAT has numerous other anti-inflammatory and tissue-protective effects, for example in pre-clinical models of diabetes, graft versus host disease and inflammatory bowel disease. The production of AAT in the lung and/or nose following transduction according to the present invention may, therefore, be more widely applicable, including to these indications.

Other examples of diseases that may be treated with gene therapy of a secreted protein according to the present invention include cardiovascular diseases and blood disorders, particularly blood clotting deficiencies such as haemophilia (A, B or C), von Willebrand disease and Factor VII deficiency.

Other examples of diseases or disorders to be treated include Primary Ciliary Dyskinesia (PCD), acute lung injury, Surfactant Protein B (SFTB) deficiency, Pulmonary Alveolar Proteinosis (PAP, hereditary and/or acquired), Chronic Obstructive Pulmonary Disease (COPD), pulmonary surfactant metabolism dysfunction 3 (SMDP3) or another surfactant deficiency, acute respiratory distress syndrome (ARDS), COVID-19, a pulmonary fibrotic disease (including idiopathic pulmonary fibrosis), a pulmonary allergic condition, asthma, lung cancer or a dysplastic change in the lungs, haemophilia and/or inflammatory, infectious, immune or metabolic conditions, such as lysosomal storage diseases or a pulmonary bacterial infection, or any other lung disease or disorder.

The nucleic acid cassettes and vectors (viral and non-viral) of the invention, particularly the retroviral/lentiviral (e.g. SIV) vectors of the present invention, typically provide high expression levels of a therapeutic protein when administered to a patient. The terms high expression and therapeutic expression are used interchangeably herein. Expression may be measured by any appropriate method (qualitative or quantitative, preferably quantitative), and concentrations given in any appropriate unit of measurement, for example ng/ml or μM.

Expression/secretion/membrane insertion of a therapeutic protein of interest may be given in absolute terms. Alternatively, expression/secretion/membrane insertion of a therapeutic protein may be given in relative terms, for example relative to the expression/secretion/membrane insertion of the therapeutic protein encoded by a corresponding nucleic acid cassette or vector of the invention without the exogenous signal peptide or with the endogenous signal peptide of the therapeutic protein or relative to the expression/secretion/membrane insertion of the corresponding endogenous (defective) gene.

Expression may be measured in terms of mRNA or protein expression. The expression of the therapeutic protein of the invention, such as a functional CFTR gene, may be quantified relative to the endogenous protein or gene, such as the endogenous (dysfunctional) CFTR genes in terms of protein concentration, mRNA copies per cell or any other appropriate unit. Secretion and/or membrane insertion of a therapeutic protein may be quantified relative to secretion/membrane insertion of the corresponding endogenous protein, or relative to the level of secretion/membrane insertion of the therapeutic protein introduced via an expression cassette lacking the exogenous signal peptide and/or comprising the endogenous signal peptide of the therapeutic protein.

Expression levels of a nucleic acid encoding a therapeutic protein and/or the expression/secretion/membrane insertion of the encoded therapeutic protein of the invention may be measured ex vivo (e.g. in the conditioned media used to culture the cells or within the cells themselves) or in vivo (e.g. in the lung tissue, epithelial lining fluid and/or serum/plasma) as appropriate. A high and/or therapeutic expression level may therefore refer to the concentration in the lung, epithelial lining fluid and/or serum/plasma.

Repeated doses of nucleic acid cassettes and vectors (viral and non-viral) of the invention, particularly the retroviral/lentiviral (e.g. SIV) vectors of the present invention may be administered twice-daily, daily, twice-weekly, weekly, monthly, every two months, every three months, every four months, every six months, yearly, every two years, or more. Dosing may be continued for as long as required, for example, for at least six months, at least one year, two years, three years, four years, five years, ten years, fifteen years, twenty years, or more, up to for the lifetime of the patient to be treated.

The invention also provides nucleic acid cassettes and vectors of the present invention, and particularly retroviral/lentiviral (e.g. SIV) vectors of the invention as described herein for use in a method of gene therapy, wherein said method comprises the steps of: (a) transducing cells (e.g. airway cells) ex vivo to produce modified cells expressing a transgene of interest; and (b) administering the resulting modified cells.

The invention provides a method of treating a disease, the method comprising administering a nucleic acid cassette or vector of the present invention, and particularly a retroviral/lentiviral (e.g. SIV) vector of the invention to a subject. Any disease described herein may be treated according to the invention. In particular, the invention provides a method of treating a lung disease using a nucleic acid cassette or vector of the present invention, and particularly a retroviral/lentiviral (e.g. SIV) vector of the invention. The disease to be treated may be a chronic disease.

The invention also provides a nucleic acid cassette or vector of the present invention, and particularly a retroviral/lentiviral (e.g. SIV) vector of the invention as described herein for use in a method of treating a disease. Any disease described herein may be treated according to the invention. In particular, the invention provides a nucleic acid cassette or vector of the present invention, and particularly a retroviral/lentiviral (e.g. SIV) vector of the invention for use in a method of treating a lung disease. The disease to be treated may be a chronic disease.

The invention also provides the use of a nucleic acid cassette or vector of the present invention, and particularly a retroviral/lentiviral (e.g. SIV) vector of the invention as described herein in the manufacture of a medicament for use in a method of treating a disease. Any disease described herein may be treated according to the invention. In particular, the invention provides the use of a nucleic acid cassette or vector of the present invention, and particularly a retroviral/lentiviral (e.g. SIV) vector of the invention for the manufacture of a medicament for use in a method of treating a lung disease. The disease to be treated may be a chronic disease.

The invention also provides a cell comprising a nucleic acid cassette or vector of the present invention, and particularly a retroviral/lentiviral (e.g. SIV) vector. Said cell may be an airway cell as described herein, or a host cell for the production of said nucleic acid cassette or vector of the present invention, as described herein.

Any and all disclosure herein in relation to nucleic acid cassettes or vectors of the present invention, and particularly retroviral/lentiviral (e.g. SIV) vectors of the invention applies equally and without reservation to the therapeutic uses and methods described herein.

Long term/persistent stable gene expression, preferably at a therapeutically-effective level, may be achieved using repeat doses of nucleic acid cassettes or vectors of the present invention, and particularly retroviral/lentiviral (e.g. SIV) vectors of the invention of the present invention. Alternatively, a single dose may be used to achieve the desired long-term expression.

### Formulation and Administration

The invention also provides a composition comprising a nucleic acid cassette or vector of the present invention, and particularly a retroviral/lentiviral (e.g. SIV) vector of the invention, and optionally a pharmaceutically acceptable carrier, excipient, buffer or diluent.

The nucleic acid cassettes or vectors of the present invention, and particularly retroviral/lentiviral (e.g. SIV) vectors of the invention may be administered in any dosage appropriate for achieving the desired therapeutic effect. Appropriate dosages may be determined by a clinician or other medical practitioner using standard techniques and within the normal course of their work. Non-limiting examples of suitable dosages of viral vectors of the invention include 1×10⁸ transduction units (TU), 1×10⁹ TU, 1×10¹⁰ TU, 1×10¹¹ TU or more. Non-limiting examples of suitable dosages of non-viral vectors/delivery means of the invention include a maximum of 30 mL per dose, a maximum of 25 mL per dose, a maximum of 20 mL per dose, a maximum of 15 mL per dose, a maximum of 10 mL per dose, or less, preferably a maximum of 20 mL per dose.

Non-limiting examples of pharmaceutically acceptable carriers that may be comprised in a composition of the invention include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long-term storage.

The nucleic acid cassettes or vectors of the present invention, and particularly retroviral/lentiviral (e.g. SIV) vectors of the invention may be administered by any appropriate route. It may be desired to direct the compositions of the present invention (as described above) to the respiratory system of a subject. Efficient transmission of a therapeutic/prophylactic composition or medicament to the site of a disease or disorder in the respiratory tract may be achieved by oral or intra-nasal administration, for example, as aerosols (e.g. nasal sprays), or by catheters. Typically the nucleic acid cassettes or vectors of the present invention, and particularly retroviral/lentiviral (e.g. SIV) vectors of the invention are stable in clinically relevant nebulisers, inhalers (including metered dose inhalers), catheters and aerosols, etc.

Other routes of administration, including but not limited to i.v. administration, intranasal administration and intrapleural injection are also encompassed by the present invention. Suitable administration routes are known in the art.

In some embodiments the nose is a preferred production site for a therapeutic protein using nucleic acid cassettes or vectors of the present invention, and particularly retroviral/lentiviral (e.g. SIV) vectors of the invention for at least one of the following reasons: (i) extracellular barriers such as inflammatory cells and sputum are less pronounced in the nose; (ii) ease of vector administration; (iii) smaller quantities of vector required; and (iv) ethical considerations. Thus, nasal administration of nucleic acid cassettes or vectors of the present invention, and particularly retroviral/lentiviral (e.g. SIV) vectors of the invention may result in efficient (high-level) and long-lasting expression of the therapeutic protein of interest. Accordingly, nasal administration of nucleic acid cassettes or vectors of the present invention, and particularly retroviral/lentiviral (e.g. SIV) vectors of the invention may be preferred.

Formulations for intra-nasal administration may be in the form of nasal droplets or a nasal spray. An intra-nasal formulation may comprise droplets having approximate diameters in the range of 100-5000 μm, such as 500-4000 μm, 1000-3000 μm or 100-1000 μm. Alternatively, in terms of volume, the droplets may be in the range of about 0.001-100 μl, such as 0.1-50 μl or 1.0-25 μl, or such as 0.001-1 μl.

The aerosol formulation may take the form of a powder, suspension or solution. The size of aerosol particles is relevant to the delivery capability of an aerosol. Smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli. In the case of aerosol delivery of the medicament, the particles may have diameters in the approximate range of 0.1-50 μm, preferably 1-25 μm, more preferably 1-5 μm.

Aerosol particles may be for delivery using a nebulizer (e.g. via the mouth) or nasal spray. An aerosol formulation may optionally contain a propellant and/or surfactant.

The formulation of pharmaceutical aerosols is routine to those skilled in the art, see for example, Sciarra, J. in Remington's Pharmaceutical Sciences (supra). The agents may be formulated as solution aerosols, dispersion or suspension aerosols of dry powders, emulsions or semisolid preparations. The aerosol may be delivered using any propellant system known to those skilled in the art. The aerosols may be applied to the upper respiratory tract, for example by nasal inhalation, or to the lower respiratory tract or to both. The part of the lung that the medicament is delivered to may be determined by the disorder. Compositions comprising nucleic acid cassettes or vectors of the present invention, and particularly retroviral/lentiviral (e.g. SIV) vectors of the invention, in particular where intranasal delivery is to be used, may comprise a humectant. This may help reduce or prevent drying of the mucus membrane and to prevent irritation of the membranes. Suitable humectants include, for instance, sorbitol, mineral oil, vegetable oil and glycerol; soothing agents; membrane conditioners; sweeteners; and combinations thereof. The compositions may comprise a surfactant. Suitable surfactants include non-ionic, anionic and cationic surfactants. Examples of surfactants that may be used include, for example, polyoxyethylene derivatives of fatty acid partial esters of sorbitol anhydrides, such as for example, Tween 80, Polyoxyl 40 Stearate, Polyoxy ethylene 50 Stearate, fusieates, bile salts and Octoxynol.

In some cases after an initial administration a subsequent administration of nucleic acid cassettes or vectors of the present invention, and particularly retroviral/lentiviral (e.g. SIV) vectors of the invention may be performed. The administration may, for instance, be at least a week, two weeks, a month, two months, six months, a year or more after the initial administration. In some instances, nucleic acid cassettes or vectors of the present invention, and particularly retroviral/lentiviral (e.g. SIV) vectors of the invention may be administered at least once a week, once a fortnight, once a month, every two months, every six months, annually or at longer intervals. Preferably, administration is every six months, more preferably annually. The nucleic acid cassettes or vectors of the present invention, and particularly retroviral/lentiviral (e.g. SIV) vectors may, for instance, be administered at intervals dictated by when the effects of the previous administration are decreasing.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### Alignment Scores for Determining Sequence Identity

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}\begin{matrix}[ length of the longer sequence plus the \\ number of gaps introduced into the longer\end{matrix} \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (as described herein) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### Sequence Information

### Key to Sequences

SEQ ID NO: 1 Exemplified CRTAC1 signal peptide
SEQ ID NO: 2 Exemplified AAT signal peptide
SEQ ID NO: 3 Exemplified CLEC3B signal peptide
SEQ ID NO: 4 Exemplified A2M signal peptide
SEQ ID NO: 5 Exemplified SCGB1A1 signal peptide
SEQ ID NO: 6 Exemplified SFTPA2 signal peptide
SEQ ID NO: 7 Exemplified GM-CSF signal peptide (human)
SEQ ID NO: 8 Exemplified GM-CSF signal peptide (mouse)
SEQ ID NO: 9 Exemplified IDS signal peptide
SEQ ID NO: 10 Exemplified Secrecon signal peptide
SEQ ID NO: 11 Exemplified Secrecon signal peptide
SEQ ID NO: 12 Exemplified Secrecon-AAT hybrid signal peptide
SEQ ID NO: 13 Exemplified hCEF promoter
SEQ ID NO: 14 Exemplified CMV promoter
SEQ ID NO: 15 Exemplified EF1a promoter
SEQ ID NO: 16 Exemplified CFTR transgene (soCFTR)
SEQ ID NO: 17 Exemplified CFTR polypeptide
SEQ ID NO: 18 Exemplified hGM-CSF transgene
SEQ ID NO: 19 Exemplified hGM-CSF polypeptide
SEQ ID NO: 20 Exemplified mGM-CSF transgene
SEQ ID NO: 21 Exemplified mGM-CSF polypeptide
SEQ ID NO: 22 Exemplified AAT transgene (SERPINA1)
SEQ ID NO: 23 Complementary strand to the exemplified AAT transgene (SERPINA2)
SEQ ID NO: 24 Exemplified A1A1 polypeptide
SEQ ID NO: 25 Exemplified FVIII transgene (N6)
SEQ ID NO: 26 Exemplified FVIII transgene (V3)
SEQ ID NO: 27 Complementary strand to the exemplified FVIII transgene (N6)
SEQ ID NO: 28 Complementary strand to the exemplified FVIII transgene (V3)
SEQ ID NO: 29 Exemplified FVIII polypeptide (N6)
SEQ ID NO: 30 Exemplified FVIII polypeptide (V3)
SEQ ID NO: 31 Exemplified Human DCN (Decorin) transgene
SEQ ID NO: 32 Exemplified Human Decorin polypeptide
SEQ ID NO: 33 Exemplified Human TRIM72 transgene
SEQ ID NO: 34 Exemplified Human TRIM72 polypeptide
SEQ ID NO: 35 Exemplified Human ABACA3 (ABCA3) transgene
SEQ ID NO: 36 Exemplified Human ABCA3 polypeptide
SEQ ID NO: 37 Exemplified WPRE component (mWPRE)

| Sequences | |
|---|---|
| Exemplified CRTAC1 signal peptide | |
| SEQ ID NO: 1 | |
| MAPSADPGMSRMLPFLLLLWFLPITEG | |
| Exemplified AAT signal peptide | |
| SEQ ID NO: 2 | |
| MPSSVSWGILLLAGLCCLVPVSLA | |
| Exemplified CLEC3B signal peptide | |
| SEQ ID NO: 3 | |
| MELWGAYLLLCLFSLLTQVTT | |
| Exemplified A2M signal peptide | |
| SEQ ID NO: 4 | |
| MGKNKLLGPSLVLLLLVLLPTDA | |
| Exemplified SCGB1A1 signal peptide | |
| SEQ ID NO: 5 | |
| MKLAVTLTLVTLALCCSSASA | |
| Exemplified SFTPA2 signal peptide | |
| SEQ ID NO: 6 | |
| MWLCPLALNLILMAASGAAC | |
| Exemplified GM-CSF signal peptide (human) | |
| SEQ ID NO: 7 | |
| MWLQSLLLLGTVACSIS | |
| Exemplified GM-CSF signal peptide (mouse) | |
| SEQ ID NO: 8 | |
| MWLQNLLFLGIVVYSLS | |
| Exemplified IDS signal peptide | |
| SEQ ID NO: 9 | |
| MPPPRTGRGLLWLGLVLSSVCVALGA | |
| Exemplified Secrecon signal peptide | |
| SEQ ID NO: 10 | |
| MWWRLWWLLLLLLLLWPMVWA | |
| Exemplified Secrecon signal peptide | |
| SEQ ID NO: 11 | |
| MWWRLWWLLLLLLLLWPMVWAAA | |
| Exemplified Secrecon-AAT hybrid signal peptide | |
| SEQ ID NO: 12 | |
| MPWWVSWWLLLLLLLCCLVPVVWAAA | |
| Exemplified hCEF promoter | |
| SEQ ID NO: 13 | |
| 1 | AGATCTGTTA CATAACTTAT GGTAAATGGC CTGCCTGGCT GACTGCCCAA TGACCCCTGC |
| 61 | CCAATGATGT CAATAATGAT GTATGTTCCC ATGTAATGCC AATAGGGACT TTCCATTGAT |
| 121 | GTCAATGGGT GGAGTATTTA TGGTAACTGC CCACTTGGCA GTACATCAAG TGTATCATAT |
| 181 | GCCAAGTATG CCCCCTATTG ATGTCAATGA TGGTAAATGG CCTGCCTGGC ATTATGCCCA |
| 241 | GTACATGACC TTATGGGACT TTCCTACTTG GCAGTACATC TATGTATTAG TCATTGCTAT |
| 301 | TACCATGGGA ATTCACTAGT GGAGAAGAGC ATGCTTGAGG GCTGAGTGCC CCTCAGTGGG |
| 361 | CAGAGAGCAC ATGGCCCACA GTCCCTGAGA AGTTGGGGGG AGGGGTGGGC AATTGAACTG |
| 421 | GTGCCTAGAG AAGGTGGGGC TTGGGTAAAC TGGGAAAGTG ATGTGGTGTA CTGGCTCCAC |
| 481 | CTTTTTCCCC AGGGTGGGGG AGAACCATAT ATAAGTGCAG TAGTCTCTGT GAACATTCAA |
| 541 | GCTTCTGCCT TCTCCCTCCT GTGAGTTTGC TAGC |
| Exemplified CMV promoter | |
| SEQ ID NO: 14 | |
| CCGCGGAGATCTCAATATTGGCCATTAGCCATATTATTCATTGGTTATATAGCATAAATCAATATTGGCT | |
| ATTGGCCATTGCATACGTTGTATCTATATCATAATATGTACATTTATATTGGCTCATGTCCAATATGACC | |
| GCCATGTTGGCATTGATTATTGACTAGTTATTAATAGTAATCAATTACGGGGTCATTAGTTCATAGCCCA | |
| TATATGGAGTTCCGCGTTACATAACTTACGGTAAATGGCCCGCCTGGCTGACCGCCCAACGACCCCCGCC | |
| CATTGACGTCAATAATGACGTATGTTCCCATAGTAACGCCAATAGGGACTTTCCATTGACGTCAATGGGT | |
| GGAGTATTTACGGTAAACTGCCCACTTGGCAGTACATCAAGTGTATCATATGCCAAGTCCGCCCCCTATT | |
| GACGTCAATGACGGTAAATGGCCCGCCTGGCATTATGCCCAGTACATGACCTTACGGGACTTTCCTACTT | |
| GGCAGTACATCTACGTATTAGTCATCGCTATTACCATGGTGATGCGGTTTTGGCAGTACACCAATGGGCG | |
| TGGATAGCGGTTTGACTCACGGGGATTTCCAAGTCTCCACCCCATTGACGTCAATGGGAGTTTGTTTTGG | |
| CACCAAAATCAACGGGACTTTCCAAAATGTCGTAATAACCCCGCCCCGTTGACGCAAATGGGCGGTAGGC | |
| GTGTACGGTGGGAGGTCTATATAAGCAGAGCTCGTTTAGTGAACCGTCAGATCACTAGAAGCTTTATTGC | |
| GGTAGTTTATCACAGTTAAATTGCTAACGCAGTCAGTGCTTCTGACACAACAGTCTCGAACTTAAGCTGC | |
| AGAAGTTGGTCGTGAGGCACTGGGCAGGCTAGC | |
| Exemplified EF1a promoter | |
| SEQ ID NO: 15 | |
| AGATCCATATCCGCGGCAATTTTAAAAGAAAGGGAGGAATAGGGGGACAGACTTCAGCAGAGAGACTAATTAATA | |
| TAATAACAACACAATTAGAAATACAACATTTACAAACCAAAATTCAAAAAATTTTAAATTTTAGAGCCGCGGAGA | |
| TCCCGTGAGGCTCCGGTGCCCGTCAGTGGGCAGAGCGCACATCGCCCACAGTCCCCGAGAAGTTGGGGGGAGGGG | |
| TCGGCAATTGAACCGGTGCCTAGAGAAGGTGGCGCGGGGTAAACTGGGAAAGTGATGTCGTGTACTGGCTCCGCC | |
| TTTTTCCCGAGGGTGGGGGAGAACCGTATATAAGTGCAGTAGTCGCCGTGAACGTTCTTTTTCGCAACGGGTTTG | |
| CCGCCAGAACACAGGCTAGC | |
| Exemplified CFTR transgene (soCFTR2) | |
| SEQ ID NO: 16 | |
| ATGCAGAGAAGCCCTCTGGAGAAGGCCTCTGTGGTGAGCAAGCTGTTCTTCAGCTGGACCAGGCCCATCCTGAGG | |
| AAGGGCTACAGGCAGAGACTGGAGCTGTCTGACATCTACCAGATCCCCTCTGTGGACTCTGCTGACAACCTGTCT | |
| GAGAAGCTGGAGAGGGAGTGGGATAGAGAGCTGGCCAGCAAGAAGAACCCCAAGCTGATCAATGCCCTGAGGAGA | |
| TGCTTCTTCTGGAGATTCATGTTCTATGGCATCTTCCTGTACCTGGGGGAAGTGACCAAGGCTGTGCAGCCTCTG | |
| CTGCTGGGCAGAATCATTGCCAGCTATGACCCTGACAACAAGGAGGAGAGGAGCATTGCCATCTACCTGGGCATT | |
| GGCCTGTGCCTGCTGTTCATTGTGAGGACCCTGCTGCTGCACCCTGCCATCTTTGGCCTGCACCACATTGGCATG | |
| CAGATGAGGATTGCCATGTTCAGCCTGATCTACAAGAAAACCCTGAAGCTGTCCAGCAGAGTGCTGGACAAGATC | |
| AGCATTGGCCAGCTGGTGAGCCTGCTGAGCAACAACCTGAACAAGTTTGATGAGGGCCTGGCCCTGGCCCACTTT | |
| GTGTGGATTGCCCCTCTGCAGGTGGCCCTGCTGATGGGCCTGATTTGGGAGCTGCTGCAGGCCTCTGCCTTTTGT | |
| GGCCTGGGCTTCCTGATTGTGCTGGCCCTGTTTCAGGCTGGCCTGGGCAGGATGATGATGAAGTACAGGGACCAG | |
| AGGGCAGGCAAGATCAGTGAGAGGCTGGTGATCACCTCTGAGATGATTGAGAACATCCAGTCTGTGAAGGCCTAC | |
| TGTTGGGAGGAAGCTATGGAGAAGATGATTGAAAACCTGAGGCAGACAGAGCTGAAGCTGACCAGGAAGGCTGCC | |
| TATGTGAGATACTTCAACAGCTCTGCCTTCTTCTTCTCTGGCTTCTTTGTGGTGTTCCTGTCTGTGCTGCCCTAT | |
| GCCCTGATCAAGGGGATCATCCTGAGAAAGATTTTCACCACCATCAGCTTCTGCATTGTGCTGAGGATGGCTGTG | |
| ACCAGACAGTTCCCCTGGGCTGTGCAGACCTGGTATGACAGCCTGGGGGCCATCAACAAGATCCAGGACTTCCTG | |
| CAGAAGCAGGAGTACAAGACCCTGGAGTACAACCTGACCACCACAGAAGTGGTGATGGAGAATGTGACAGCCTTC | |
| TGGGAGGAGGGCTTTGGGGAGCTGTTTGAGAAGGCCAAGCAGAACAACAACAACAGAAAGACCAGCAATGGGGAT | |
| GACTCCCTGTTCTTCTCCAACTTCTCCCTGCTGGGCACACCTGTGCTGAAGGACATCAACTTCAAGATTGAGAGG | |
| GGGCAGCTGCTGGCTGTGGCTGGATCTACAGGGGCTGGCAAGACCAGCCTGCTGATGATGATCATGGGGGAGCTG | |
| GAGCCTTCTGAGGGCAAGATCAAGCACTCTGGCAGGATCAGCTTTTGCAGCCAGTTCAGCTGGATCATGCCTGGC | |
| ACCATCAAGGAGAACATCATCTTTGGAGTGAGCTATGATGAGTACAGATACAGGAGTGTGATCAAGGCCTGCCAG | |
| CTGGAGGAGGACATCAGCAAGTTTGCTGAGAAGGACAACATTGTGCTGGGGGAGGGAGGCATTACACTGTCTGGG | |
| GGCCAGAGAGCCAGAATCAGCCTGGCCAGGGCTGTGTACAAGGATGCTGACCTGTACCTGCTGGACTCCCCCTTT | |
| GGCTACCTGGATGTGCTGACAGAGAAGGAGATTTTTGAGAGCTGTGTGTGCAAGCTGATGGCCAACAAGACCAGA | |
| ATCCTGGTGACCAGCAAGATGGAGCACCTGAAGAAGGCTGACAAGATCCTGATCCTGCATGAGGGCAGCAGCTAC | |
| TTCTATGGGACCTTCTCTGAGCTGCAGAACCTGCAGCCTGACTTCAGCTCTAAGCTGATGGGCTGTGACAGCTTT | |
| GACCAGTTCTCTGCTGAGAGGAGGAACAGCATCCTGACAGAGACCCTGCACAGATTCAGCCTGGAGGGAGATGCC | |
| CCTGTGAGCTGGACAGAGACCAAGAAGCAGAGCTTCAAGCAGACAGGGGAGTTTGGGGAGAAGAGGAAGAACTCC | |
| ATCCTGAACCCCATCAACAGCATCAGGAAGTTCAGCATTGTGCAGAAAACCCCCCTGCAGATGAATGGCATTGAG | |
| GAAGATTCTGATGAGCCCCTGGAGAGGAGACTGAGCCTGGTGCCTGATTCTGAGCAGGGAGAGGCCATCCTGCCT | |
| AGGATCTCTGTGATCAGCACAGGCCCTACACTGCAGGCCAGAAGGAGGCAGTCTGTGCTGAACCTGATGACCCAC | |
| TCTGTGAACCAGGGCCAGAACATCCACAGGAAAACCACAGCCTCCACCAGGAAAGTGAGCCTGGCCCCTCAGGCC | |
| AATCTGACAGAGCTGGACATCTACAGCAGGAGGCTGTCTCAGGAGACAGGCCTGGAGATTTCTGAGGAGATCAAT | |
| GAGGAGGACCTGAAAGAGTGCTTCTTTGATGACATGGAGAGCATCCCTGCTGTGACCACCTGGAACACCTACCTG | |
| AGATACATCACAGTGCACAAGAGCCTGATCTTTGTGCTGATCTGGTGCCTGGTGATCTTCCTGGCTGAAGTGGCT | |
| GCCTCTCTGGTGGTGCTGTGGCTGCTGGGAAACACCCCACTGCAGGACAAGGGCAACAGCACCCACAGCAGGAAC | |
| AACAGCTATGCTGTGATCATCACCTCCACCTCCAGCTACTATGTGTTCTACATCTATGTGGGAGTGGCTGATACC | |
| CTGCTGGCTATGGGCTTCTTTAGAGGCCTGCCCCTGGTGCACACACTGATCACAGTGAGCAAGATCCTCCACCAC | |
| AAGATGCTGCACTCTGTGCTGCAGGCTCCTATGAGCACCCTGAATACCCTGAAGGCTGGGGGCATCCTGAACAGA | |
| TTCTCCAAGGATATTGCCATCCTGGATGACCTGCTGCCTCTCACCATCTTTGACTTCATCCAGCTGCTGCTGATT | |
| GTGATTGGGGCCATTGCTGTGGTGGCAGTGCTGCAGCCCTACATCTTTGTGGCCACAGTGCCTGTGATTGTGGCC | |
| TTCATCATGCTGAGGGCCTACTTTCTGCAGACCTCCCAGCAGCTGAAGCAGCTGGAGTCTGAGGGCAGAAGCCCC | |
| ATCTTCACCCACCTGGTGACAAGCCTGAAGGGCCTGTGGACCCTGAGAGCCTTTGGCAGGCAGCCCTACTTTGAG | |
| ACCCTGTTCCACAAGGCCCTGAACCTGCACACAGCCAACTGGTTCCTCTACCTGTCCACCCTGAGATGGTTCCAG | |
| ATGAGAATTGAGATGATCTTTGTCATCTTCTTCATTGCTGTGACCTTCATCAGCATTCTGACCACAGGAGAGGGA | |
| GAGGGCAGAGTGGGCATTATCCTGACCCTGGCCATGAACATCATGAGCACACTGCAGTGGGCAGTGAACAGCAGC | |
| ATTGATGTGGACAGCCTGATGAGGAGTGTGAGCAGAGTGTTCAAGTTCATTGATATGCCCACAGAGGGCAAGCCT | |
| ACCAAGAGCACCAAGCCCTACAAGAATGGCCAGCTGAGCAAAGTGATGATCATTGAGAACAGCCATGTGAAGAAG | |
| GATGATATCTGGCCCAGTGGAGGCCAGATGACAGTGAAGGACCTGACAGCCAAGTACACAGAGGGGGGCAATGCT | |
| ATCCTGGAGAACATCTCCTTCAGCATCTCCCCTGGCCAGAGAGTGGGACTGCTGGGAAGAACAGGCTCTGGCAAG | |
| TCTACCCTGCTGTCTGCCTTCCTGAGGCTGCTGAACACAGAGGGAGAGATCCAGATTGATGGAGTGTCCTGGGAC | |
| AGCATCACACTGCAGCAGTGGAGGAAGGCCTTTGGTGTGATCCCCCAGAAAGTGTTCATCTTCAGTGGCACCTTC | |
| AGGAAGAACCTGGACCCCTATGAGCAGTGGTCTGACCAGGAGATTTGGAAAGTGGCTGATGAAGTGGGCCTGAGA | |
| AGTGTGATTGAGCAGTTCCCTGGCAAGCTGGACTTTGTCCTGGTGGATGGGGGCTGTGTGCTGAGCCATGGCCAC | |
| AAGCAGCTGATGTGCCTGGCCAGATCAGTGCTGAGCAAGGCCAAGATCCTGCTGCTGGATGAGCCTTCTGCCCAC | |
| CTGGATCCTGTGACCTACCAGATCATCAGGAGGACCCTCAAGCAGGCCTTTGCTGACTGCACAGTCATCCTGTGT | |
| GAGCACAGGATTGAGGCCATGCTGGAGTGCCAGCAGTTCCTGGTGATTGAGGAGAACAAAGTGAGGCAGTATGAC | |
| AGCATCCAGAAGCTGCTGAATGAGAGGAGCCTGTTCAGGCAGGCCATCAGCCCCTCTGATAGAGTGAAGCTGTTC | |
| CCCCACAGGAACAGCTCCAAGTGCAAGAGCAAGCCCCAGATTGCTGCCCTGAAGGAGGAGACAGAGGAGGAAGTG | |
| CAGGACACCAGGCTGTGA | |
| Exemplified CFTR polypeptide | |
| SEQ ID NO: 17 | |
| MQRSPLEKASVVSKLFFSWTRPILRKGYRQRLELSDIYQIPSVDSADNLSEKLEREWDRELASKKNPKLINALRR | |
| CFFWRFMFYGIFLYLGEVTKAVQPLLLGRIIASYDPDNKEERSIAIYLGIGLCLLFIVRTLLLHPAIFGLHHIGM | |
| QMRIAMFSLIYKKTLKLSSRVLDKISIGQLVSLLSNNLNKFDEGLALAHFVWIAPLQVALLMGLIWELLQASAFC | |
| GLGFLIVLALFQAGLGRMMMKYRDQRAGKISERLVITSEMIENIQSVKAYCWEEAMEKMIENLRQTELKLTRKAA | |
| YVRYFNSSAFFFSGFFVVFLSVLPYALIKGIILRKIFTTISFCIVLRMAVTRQFPWAVQTWYDSLGAINKIQDEL | |
| QKQEYKTLEYNLTTTEVVMENVTAFWEEGFGELFEKAKQNNNNRKTSNGDDSLFFSNFSLLGTPVLKDINFKIER | |
| GQLLAVAGSTGAGKTSLLMMIMGELEPSEGKIKHSGRISFCSQFSWIMPGTIKENIIFGVSYDEYRYRSVIKACQ | |
| LEEDISKFAEKDNIVLGEGGITLSGGQRARISLARAVYKDADLYLLDSPFGYLDVLTEKEIFESCVCKLMANKTR | |
| ILVTSKMEHLKKADKILILHEGSSYFYGTFSELQNLQPDFSSKLMGCDSFDQFSAERRNSILTETLHRFSLEGDA | |
| PVSWTETKKQSFKQTGEFGEKRKNSILNPINSIRKESIVQKTPLQMNGIEEDSDEPLERRLSLVPDSEQGEAILP | |
| RISVISTGPTLQARRRQSVLNLMTHSVNQGQNIHRKTTASTRKVSLAPQANLTELDIYSRRLSQETGLEISEEIN | |
| EEDLKECFFDDMESIPAVTTWNTYLRYITVHKSLIFVLIWCLVIFLAEVAASLVVLWLLGNTPLQDKGNSTHSRN | |
| NSYAVIITSTSSYYVFYIYVGVADTLLAMGFFRGLPLVHTLITVSKILHHKMLHSVLQAPMSTLNTLKAGGILNR | |
| FSKDIAILDDLLPLTIFDFIQLLLIVIGAIAVVAVLQPYIFVATVPVIVAFIMLRAYFLQTSQQLKQLESEGRSP | |
| IFTHLVTSLKGLWTLRAFGRQPYFETLFHKALNLHTANWFLYLSTLRWFQMRIEMIFVIFFIAVTFISILTTGEG | |
| EGRVGIILTLAMNIMSTLQWAVNSSIDVDSLMRSVSRVFKFIDMPTEGKPTKSTKPYKNGQLSKVMIIENSHVKK | |
| DDIWPSGGQMTVKDLTAKYTEGGNAILENISFSISPGQRVGLLGRTGSGKSTLLSAFLRLLNTEGEIQIDGVSWD | |
| SITLQQWRKAFGVIPQKVFIFSGTFRKNLDPYEQWSDQEIWKVADEVGLRSVIEQFPGKLDFVLVDGGCVLSHGH | |
| KQLMCLARSVLSKAKILLLDEPSAHLDPVTYQIIRRTLKQAFADCTVILCEHRIEAMLECQQFLVIEENKVRQYD | |
| SIQKLLNERSLFRQAISPSDRVKLFPHRNSSKCKSKPQIAALKEETEEEVQDTRL | |
| Exemplified Human GM-CSF (CSF2) transgene | |
| SEQ ID NO: 18 | |
| ATGTGGCTGCAGAGCCTGCTGCTCTTGGGCACTGTGGCCTGCAGCATCTCTGCACCCGCCCGCTCGCCCAGCCCC | |
| AGCACGCAGCCCTGGGAGCATGTGAATGCCATCCAGGAGGCCCGGCGTCTCCTGAACCTGAGTAGAGACACTGCT | |
| GCTGAGATGAATGAAACAGTAGAAGTCATCTCAGAAATGTTTGACCTCCAGGAGCCGACCTGCCTACAGACCCGC | |
| CTGGAGCTGTACAAGCAGGGCCTGCGGGGCAGCCTCACCAAGCTCAAGGGCCCCTTGACCATGATGGCCAGCCAC | |
| TACAAGCAGCACTGCCCTCCAACCCCGGAAACTTCCTGTGCAACCCAGATTATCACCTTTGAAAGTTTCAAAGAG | |
| AACCTGAAGGACTTTCTGCTTGTCATCCCCTTTGACTGCTGGGAGCCAGTCCAGGAGTGA | |
| Nucleic acid sequence encoding signal peptide is underlined. | |
| Exemplified Human GM-CSF polypeptide | |
| SEQ ID NO: 19 | |
| MWLQSLLLLGTVACSISAPARSPSPSTQPWEHVNAIQEARRLLNLSRDTAAEMNETVEVISEMFDLQEPTCLQTR | |
| LELYKQGLRGSLTKLKGPLTMMASHYKQHCPPTPETSCATQIITFESFKENLKDELLVIPFDCWEPVQE | |
| signal peptide is underlined. | |
| Exemplified Mouse GM-CSF (CSF2) transgene | |
| SEQ ID NO: 20 | |
| ATGTGGCTGCAGAACCTGCTGTTCCTGGGCATTGTGGTGTACAGCCTGTCTGCCCCTACAAGATCCCCTATCACA | |
| GTGACCAGACCTTGGAAACATGTGGAAGCCATCAAAGAGGCCCTGAATCTGCTGGATGACATGCCTGTGACACTG | |
| AATGAAGAGGTGGAAGTGGTGTCCAATGAGTTCAGCTTCAAGAAACTGACCTGTGTGCAGACCAGGCTGAAGATT | |
| TTTGAGCAGGGCCTGAGAGGCAACTTCACCAAGCTGAAAGGGGCTCTGAACATGACAGCCAGCTACTACCAGACC | |
| TACTGTCCTCCTACACCTGAGACAGACTGTGAAACCCAAGTGACCACCTATGCTGACTTCATTGACAGCCTCAAG | |
| ACCTTCCTGACAGACATCCCCTTTGAGTGCAAGAAACCTGGCCAGAAGTGA | |
| Nucleic acid sequence encoding signal peptide is underlined. | |
| Exemplified Mouse GM-CSF polypeptide | |
| SEQ ID NO: 21 | |
| MWLQNLLFLGIVVYSLSAPTRSPITVTRPWKHVEAIKEALNLLDDMPVTLNEEVEVVSNEFSFKKLTCVQTRLKI | |
| FEQGLRGNFTKLKGALNMTASYYQTYCPPTPETDCETQVTTYADFIDSLKTFLTDIPFECKKPGQK | |
| signal peptide is underlined. | |
| Exemplified SERPINA1 (AAT) transgene | |
| SEQ ID NO: 22 | |
| ATGCCCAGCTCTGTGTCCTGGGGCATTCTGCTGCTGGCTGGCCTGTGCTGTCTGGTGCCTGTGTCCCTGG | |
| CTGAGGACCCTCAGGGGGATGCTGCCCAGAAAACAGACACCTCCCACCATGACCAGGACCACCCCACCTT | |
| CAACAAGATCACCCCCAACCTGGCAGAGTTTGCCTTCAGCCTGTACAGACAGCTGGCCCACCAGAGCAAC | |
| AGCACCAACATCTTTTTCAGCCCTGTGTCCATTGCCACAGCCTTTGCCATGCTGAGCCTGGGCACCAAGG | |
| CTGACACCCATGATGAGATCCTGGAAGGCCTGAACTTCAACCTGACAGAGATCCCTGAGGCCCAGATCCA | |
| TGAGGGCTTCCAGGAACTGCTGAGAACCCTGAACCAGCCAGACAGCCAGCTGCAGCTGACAACAGGCAAT | |
| GGGCTGTTCCTGTCTGAGGGCCTGAAGCTGGTGGACAAGTTTCTGGAAGATGTGAAGAAGCTGTACCACT | |
| CTGAGGCCTTCACAGTGAACTTTGGGGACACAGAAGAGGCCAAGAAACAGATCAATGACTATGTGGAAAA | |
| GGGCACCCAGGGCAAGATTGTGGACCTTGTGAAAGAGCTGGACAGGGACACTGTGTTTGCCCTTGTGAAC | |
| TACATCTTCTTCAAGGGCAAGTGGGAGAGGCCCTTTGAAGTGAAGGACACTGAGGAAGAGGACTTCCATG | |
| TGGACCAAGTGACCACAGTGAAGGTGCCAATGATGAAGAGACTGGGGATGTTCAATATCCAGCACTGCAA | |
| GAAACTGAGCAGCTGGGTGCTGCTGATGAAGTACCTGGGCAATGCTACAGCCATATTCTTTCTGCCTGAT | |
| GAGGGCAAGCTGCAGCACCTGGAAAATGAGCTGACCCATGACATCATCACCAAATTTCTGGAAAATGAGG | |
| ACAGAAGATCTGCCAGCCTGCATCTGCCCAAGCTGAGCATCACAGGCACATATGACCTGAAGTCTGTGCT | |
| GGGACAGCTGGGAATCACCAAGGTGTTCAGCAATGGGGCAGACCTGAGTGGAGTGACAGAGGAAGCCCCT | |
| CTGAAGCTGTCCAAGGCTGTGCACAAGGCAGTGCTGACCATTGATGAGAAGGGCACAGAGGCTGCTGGGG | |
| CCATGTTTCTGGAAGCCATCCCCATGTCCATCCCCCCAGAAGTGAAGTTCAACAAGCCCTTTGTGTTCCT | |
| GATGATTGAGCAGAACACCAAGAGCCCCCTGTTCATGGGCAAGGTTGTGAACCCCACCCAGAAATGA | |
| Nucleic acid sequence encoding signal peptide is underlined. | |
| Complementary strand to the exemplified SERPINA1 (AAT) transgene | |
| SEQ ID NO: 23 | |
| TACGGGTCGAGACACAGGACCCCGTAAGACGACGACCGACCGGACACGACAGACCACGGACACAGGGACC | |
| GACTCCTGGGAGTCCCCCTACGACGGGTCTTTTGTCTGTGGAGGGTGGTACTGGTCCTGGTGGGGTGGAA | |
| GTTGTTCTAGTGGGGGTTGGACCGTCTCAAACGGAAGTCGGACATGTCTGTCGACCGGGTGGTCTCGTTG | |
| TCGTGGTTGTAGAAAAAGTCGGGACACAGGTAACGGTGTCGGAAACGGTACGACTCGGACCCGTGGTTCC | |
| GACTGTGGGTACTACTCTAGGACCTTCCGGACTTGAAGTTGGACTGTCTCTAGGGACTCCGGGTCTAGGT | |
| ACTCCCGAAGGTCCTTGACGACTCTTGGGACTTGGTCGGTCTGTCGGTCGACGTCGACTGTTGTCCGTTA | |
| CCCGACAAGGACAGACTCCCGGACTTCGACCACCTGTTCAAAGACCTTCTACACTTCTTCGACATGGTGA | |
| GACTCCGGAAGTGTCACTTGAAACCCCTGTGTCTTCTCCGGTTCTTTGTCTAGTTACTGATACACCTTTT | |
| CCCGTGGGTCCCGTTCTAACACCTGGAACACTTTCTCGACCTGTCCCTGTGACACAAACGGGAACACTTG | |
| ATGTAGAAGAAGTTCCCGTTCACCCTCTCCGGGAAACTTCACTTCCTGTGACTCCTTCTCCTGAAGGTAC | |
| ACCTGGTTCACTGGTGTCACTTCCACGGTTACTACTTCTCTGACCCCTACAAGTTATAGGTCGTGACGTT | |
| CTTTGACTCGTCGACCCACGACGACTACTTCATGGACCCGTTACGATGTCGGTATAAGAAAGACGGACTA | |
| CTCCCGTTCGACGTCGTGGACCTTTTACTCGACTGGGTACTGTAGTAGTGGTTTAAAGACCTTTTACTCC | |
| TGTCTTCTAGACGGTCGGACGTAGACGGGTTCGACTCGTAGTGTCCGTGTATACTGGACTTCAGACACGA | |
| CCCTGTCGACCCTTAGTGGTTCCACAAGTCGTTACCCCGTCTGGACTCACCTCACTGTCTCCTTCGGGGA | |
| GACTTCGACAGGTTCCGACACGTGTTCCGTCACGACTGGTAACTACTCTTCCCGTGTCTCCGACGACCCC | |
| GGTACAAAGACCTTCGGTAGGGGTACAGGTAGGGGGGTCTTCACTTCAAGTTGTTCGGGAAACACAAGGA | |
| CTACTAACTCGTCTTGTGGTTCTCGGGGGACAAGTACCCGTTCCAACACTTGGGGTGGGTCTTTACT | |
| Exemplified AAT polypeptide | |
| SEQ ID NO: 24 | |
| MPSSVSWGILLLAGLCCLVPVSLAEDPQGDAAQKTDTSHHDQDHPTFAEDPQGDAAQKTDTSHHDQDHPTF | |
| NKITPNLAEFAFSLYRQLAHQSNSTNIFFSPVSIATAFAMLSLGTKADTHDEILEGLNFNLTEIPEAQTHE | |
| GFQELLRTLNQPDSQLQLTTGNGLFLSEGLKLVDKFLEDVKKLYHSEAFTVNFGDTEEAKKQINDYVEKGT | |
| QGKIVDLVKELDRDTVFALVNYIFFKGKWERPFEVKDTEEEDFHVDQVTTVKVPMMKRLGMFNIQHCKKLS | |
| SWVLLMKYLGNATAIFFLPDEGKLQHLENELTHDIITKFLENEDRRSASLHLPKLSITGTYDLKSVLGQLG | |
| ITKVFSNGADLSGVTEEAPLKLSKAVHKAVLTIDEKGTEAAGAMFLEAIPMSIPPEVKFNKPFVELMIEQN | |
| TKSPLFMGKVVNPTQK | |
| signal peptide is underlined. | |
| Exemplified FVIII transgene (N6) | |
| SEQ ID NO: 25 | |
| ATGCAGATTGAGCTGAGCACCTGCTTCTTCCTGTGCCTGCTGAGGTTCTGCTTCTCTGCCACCAGGAGAT | |
| ACTACCTGGGGGCTGTGGAGCTGAGCTGGGACTACATGCAGTCTGACCTGGGGGAGCTGCCTGTGGATGC | |
| CAGGTTCCCCCCCAGAGTGCCCAAGAGCTTCCCCTTCAACACCTCTGTGGTGTACAAGAAGACCCTGTTT | |
| GTGGAGTTCACTGACCACCTGTTCAACATTGCCAAGCCCAGGCCCCCCTGGATGGGCCTGCTGGGCCCCA | |
| CCATCCAGGCTGAGGTGTATGACACTGTGGTGATCACCCTGAAGAACATGGCCAGCCACCCTGTGAGCCT | |
| GCATGCTGTGGGGGTGAGCTACTGGAAGGCCTCTGAGGGGGCTGAGTATGATGACCAGACCAGCCAGAGG | |
| GAGAAGGAGGATGACAAGGTGTTCCCTGGGGGCAGCCACACCTATGTGTGGCAGGTGCTGAAGGAGAATG | |
| GCCCCATGGCCTCTGACCCCCTGTGCCTGACCTACAGCTACCTGAGCCATGTGGACCTGGTGAAGGACCT | |
| GAACTCTGGCCTGATTGGGGCCCTGCTGGTGTGCAGGGAGGGCAGCCTGGCCAAGGAGAAGACCCAGACC | |
| CTGCACAAGTTCATCCTGCTGTTTGCTGTGTTTGATGAGGGCAAGAGCTGGCACTCTGAAACCAAGAACA | |
| GCCTGATGCAGGACAGGGATGCTGCCTCTGCCAGGGCCTGGCCCAAGATGCACACTGTGAATGGCTATGT | |
| GAACAGGAGCCTGCCTGGCCTGATTGGCTGCCACAGGAAGTCTGTGTACTGGCATGTGATTGGCATGGGC | |
| ACCACCCCTGAGGTGCACAGCATCTTCCTGGAGGGCCACACCTTCCTGGTCAGGAACCACAGGCAGGCCA | |
| GCCTGGAGATCAGCCCCATCACCTTCCTGACTGCCCAGACCCTGCTGATGGACCTGGGCCAGTTCCTGCT | |
| GTTCTGCCACATCAGCAGCCACCAGCATGATGGCATGGAGGCCTATGTGAAGGTGGACAGCTGCCCTGAG | |
| GAGCCCCAGCTGAGGATGAAGAACAATGAGGAGGCTGAGGACTATGATGATGACCTGACTGACTCTGAGA | |
| TGGATGTGGTGAGGTTTGATGATGACAACAGCCCCAGCTTCATCCAGATCAGGTCTGTGGCCAAGAAGCA | |
| CCCCAAGACCTGGGTGCACTACATTGCTGCTGAGGAGGAGGACTGGGACTATGCCCCCCTGGTGCTGGCC | |
| CCTGATGACAGGAGCTACAAGAGCCAGTACCTGAACAATGGCCCCCAGAGGATTGGCAGGAAGTACAAGA | |
| AGGTCAGGTTCATGGCCTACACTGATGAAACCTTCAAGACCAGGGAGGCCATCCAGCATGAGTCTGGCAT | |
| CCTGGGCCCCCTGCTGTATGGGGAGGTGGGGGACACCCTGCTGATCATCTTCAAGAACCAGGCCAGCAGG | |
| CCCTACAACATCTACCCCCATGGCATCACTGATGTGAGGCCCCTGTACAGCAGGAGGCTGCCCAAGGGGG | |
| TGAAGCACCTGAAGGACTTCCCCATCCTGCCTGGGGAGATCTTCAAGTACAAGTGGACTGTGACTGTGGA | |
| GGATGGCCCCACCAAGTCTGACCCCAGGTGCCTGACCAGATACTACAGCAGCTTTGTGAACATGGAGAGG | |
| GACCTGGCCTCTGGCCTGATTGGCCCCCTGCTGATCTGCTACAAGGAGTCTGTGGACCAGAGGGGCAACC | |
| AGATCATGTCTGACAAGAGGAATGTGATCCTGTTCTCTGTGTTTGATGAGAACAGGAGCTGGTACCTGAC | |
| TGAGAACATCCAGAGGTTCCTGCCCAACCCTGCTGGGGTGCAGCTGGAGGACCCTGAGTTCCAGGCCAGC | |
| AACATCATGCACAGCATCAATGGCTATGTGTTTGACAGCCTGCAGCTGTCTGTGTGCCTGCATGAGGTGG | |
| CCTACTGGTACATCCTGAGCATTGGGGCCCAGACTGACTTCCTGTCTGTGTTCTTCTCTGGCTACACCTT | |
| CAAGCACAAGATGGTGTATGAGGACACCCTGACCCTGTTCCCCTTCTCTGGGGAGACTGTGTTCATGAGC | |
| ATGGAGAACCCTGGCCTGTGGATTCTGGGCTGCCACAACTCTGACTTCAGGAACAGGGGCATGACTGCCC | |
| TGCTGAAAGTCTCCAGCTGTGACAAGAACACTGGGGACTACTATGAGGACAGCTATGAGGACATCTCTGC | |
| CTACCTGCTGAGCAAGAACAATGCCATTGAGCCCAGGAGCTTCAGCCAGAACAGCAGGCACCCCAGCACC | |
| AGGCAGAAGCAGTTCAATGCCACCACCATCCCTGAGAATGACATAGAGAAGACAGACCCATGGTTTGCCC | |
| ACCGGACCCCCATGCCCAAGATCCAGAATGTGAGCAGCTCTGACCTGCTGATGCTGCTGAGGCAGAGCCC | |
| CACCCCCCATGGCCTGAGCCTGTCTGACCTGCAGGAGGCCAAGTATGAAACCTTCTCTGATGACCCCAGC | |
| CCTGGGGCCATTGACAGCAACAACAGCCTGTCTGAGATGACCCACTTCAGGCCCCAGCTGCACCACTCTG | |
| GGGACATGGTGTTCACCCCTGAGTCTGGCCTGCAGCTGAGGCTGAATGAGAAGCTGGGCACCACTGCTGC | |
| CACTGAGCTGAAGAAGCTGGACTTCAAAGTCTCCAGCACCAGCAACAACCTGATCAGCACCATCCCCTCT | |
| GACAACCTGGCTGCTGGCACTGACAACACCAGCAGCCTGGGCCCCCCCAGCATGCCTGTGCACTATGACA | |
| GCCAGCTGGACACCACCCTGTTTGGCAAGAAGAGCAGCCCCCTGACTGAGTCTGGGGGCCCCCTGAGCCT | |
| GTCTGAGGAGAACAATGACAGCAAGCTGCTGGAGTCTGGCCTGATGAACAGCCAGGAGAGCAGCTGGGGC | |
| AAGAATGTGAGCAGCAGGGAGATCACCAGGACCACCCTGCAGTCTGACCAGGAGGAGATTGACTATGATG | |
| ACACCATCTCTGTGGAGATGAAGAAGGAGGACTTTGACATCTACGACGAGGACGAGAACCAGAGCCCCAG | |
| GAGCTTCCAGAAGAAGACCAGGCACTACTTCATTGCTGCTGTGGAGAGGCTGTGGGACTATGGCATGAGC | |
| AGCAGCCCCCATGTGCTGAGGAACAGGGCCCAGTCTGGCTCTGTGCCCCAGTTCAAGAAGGTGGTGTTCC | |
| AGGAGTTCACTGATGGCAGCTTCACCCAGCCCCTGTACAGAGGGGAGCTGAATGAGCACCTGGGCCTGCT | |
| GGGCCCCTACATCAGGGCTGAGGTGGAGGACAACATCATGGTGACCTTCAGGAACCAGGCCAGCAGGCCC | |
| TACAGCTTCTACAGCAGCCTGATCAGCTATGAGGAGGACCAGAGGCAGGGGGCTGAGCCCAGGAAGAACT | |
| TTGTGAAGCCCAATGAAACCAAGACCTACTTCTGGAAGGTGCAGCACCACATGGCCCCCACCAAGGATGA | |
| GTTTGACTGCAAGGCCTGGGCCTACTTCTCTGATGTGGACCTGGAGAAGGATGTGCACTCTGGCCTGATT | |
| GGCCCCCTGCTGGTGTGCCACACCAACACCCTGAACCCTGCCCATGGCAGGCAGGTGACTGTGCAGGAGT | |
| TTGCCCTGTTCTTCACCATCTTTGATGAAACCAAGAGCTGGTACTTCACTGAGAACATGGAGAGGAACTG | |
| CAGGGCCCCCTGCAACATCCAGATGGAGGACCCCACCTTCAAGGAGAACTACAGGTTCCATGCCATCAAT | |
| GGCTACATCATGGACACCCTGCCTGGCCTGGTGATGGCCCAGGACCAGAGGATCAGGTGGTACCTGCTGA | |
| GCATGGGCAGCAATGAGAACATCCACAGCATCCACTTCTCTGGCCATGTGTTCACTGTGAGGAAGAAGGA | |
| GGAGTACAAGATGGCCCTGTACAACCTGTACCCTGGGGTGTTTGAGACTGTGGAGATGCTGCCCAGCAAG | |
| GCTGGCATCTGGAGGGTGGAGTGCCTGATTGGGGAGCACCTGCATGCTGGCATGAGCACCCTGTTCCTGG | |
| TGTACAGCAACAAGTGCCAGACCCCCCTGGGCATGGCCTCTGGCCACATCAGGGACTTCCAGATCACTGC | |
| CTCTGGCCAGTATGGCCAGTGGGCCCCCAAGCTGGCCAGGCTGCACTACTCTGGCAGCATCAATGCCTGG | |
| AGCACCAAGGAGCCCTTCAGCTGGATCAAGGTGGACCTGCTGGCCCCCATGATCATCCATGGCATCAAGA | |
| CCCAGGGGGCCAGGCAGAAGTTCAGCAGCCTGTACATCAGCCAGTTCATCATCATGTACAGCCTGGATGG | |
| CAAGAAGTGGCAGACCTACAGGGGCAACAGCACTGGCACCCTGATGGTGTTCTTTGGCAATGTGGACAGC | |
| TCTGGCATCAAGCACAACATCTTCAACCCCCCCATCATTGCCAGATACATCAGGCTGCACCCCACCCACT | |
| ACAGCATCAGGAGCACCCTGAGGATGGAGCTGATGGGCTGTGACCTGAACAGCTGCAGCATGCCCCTGGG | |
| CATGGAGAGCAAGGCCATCTCTGATGCCCAGATCACTGCCAGCAGCTACTTCACCAACATGTTTGCCACC | |
| TGGAGCCCCAGCAAGGCCAGGCTGCACCTGCAGGGCAGGAGCAATGCCTGGAGGCCCCAGGTCAACAACC | |
| CCAAGGAGTGGCTGCAGGTGGACTTCCAGAAGACCATGAAGGTGACTGGGGTGACCACCCAGGGGGTGAA | |
| GAGCCTGCTGACCAGCATGTATGTGAAGGAGTTCCTGATCAGCAGCAGCCAGGATGGCCACCAGTGGACC | |
| CTGTTCTTCCAGAATGGCAAGGTGAAGGTGTTCCAGGGCAACCAGGACAGCTTCACCCCTGTGGTGAACA | |
| GCCTGGACCCCCCCCTGCTGACCAGATACCTGAGGATTCACCCCCAGAGCTGGGTGCACCAGATTGCCCT | |
| GAGGATGGAGGTGCTGGGCTGTGAGGCCCAGGACCTGTACTGA | |
| Nucleic acid sequence encoding signal peptide is underlined. | |
| Exemplified FVIII transgene (V3) | |
| SEQ ID NO: 26 | |
| ATGCAGATTGAGCTGAGCACCTGCTTCTTCCTGTGCCTGCTGAGGTTCTGCTTCTCTGCCACCAGGAGAT | |
| ACTACCTGGGGGCTGTGGAGCTGAGCTGGGACTACATGCAGTCTGACCTGGGGGAGCTGCCTGTGGATGC | |
| CAGGTTCCCCCCCAGAGTGCCCAAGAGCTTCCCCTTCAACACCTCTGTGGTGTACAAGAAGACCCTGTTT | |
| GTGGAGTTCACTGACCACCTGTTCAACATTGCCAAGCCCAGGCCCCCCTGGATGGGCCTGCTGGGCCCCA | |
| CCATCCAGGCTGAGGTGTATGACACTGTGGTGATCACCCTGAAGAACATGGCCAGCCACCCTGTGAGCCT | |
| GCATGCTGTGGGGGTGAGCTACTGGAAGGCCTCTGAGGGGGCTGAGTATGATGACCAGACCAGCCAGAGG | |
| GAGAAGGAGGATGACAAGGTGTTCCCTGGGGGCAGCCACACCTATGTGTGGCAGGTGCTGAAGGAGAATG | |
| GCCCCATGGCCTCTGACCCCCTGTGCCTGACCTACAGCTACCTGAGCCATGTGGACCTGGTGAAGGACCT | |
| GAACTCTGGCCTGATTGGGGCCCTGCTGGTGTGCAGGGAGGGCAGCCTGGCCAAGGAGAAGACCCAGACC | |
| CTGCACAAGTTCATCCTGCTGTTTGCTGTGTTTGATGAGGGCAAGAGCTGGCACTCTGAAACCAAGAACA | |
| GCCTGATGCAGGACAGGGATGCTGCCTCTGCCAGGGCCTGGCCCAAGATGCACACTGTGAATGGCTATGT | |
| GAACAGGAGCCTGCCTGGCCTGATTGGCTGCCACAGGAAGTCTGTGTACTGGCATGTGATTGGCATGGGC | |
| ACCACCCCTGAGGTGCACAGCATCTTCCTGGAGGGCCACACCTTCCTGGTCAGGAACCACAGGCAGGCCA | |
| GCCTGGAGATCAGCCCCATCACCTTCCTGACTGCCCAGACCCTGCTGATGGACCTGGGCCAGTTCCTGCT | |
| GTTCTGCCACATCAGCAGCCACCAGCATGATGGCATGGAGGCCTATGTGAAGGTGGACAGCTGCCCTGAG | |
| GAGCCCCAGCTGAGGATGAAGAACAATGAGGAGGCTGAGGACTATGATGATGACCTGACTGACTCTGAGA | |
| TGGATGTGGTGAGGTTTGATGATGACAACAGCCCCAGCTTCATCCAGATCAGGTCTGTGGCCAAGAAGCA | |
| CCCCAAGACCTGGGTGCACTACATTGCTGCTGAGGAGGAGGACTGGGACTATGCCCCCCTGGTGCTGGCC | |
| CCTGATGACAGGAGCTACAAGAGCCAGTACCTGAACAATGGCCCCCAGAGGATTGGCAGGAAGTACAAGA | |
| AGGTCAGGTTCATGGCCTACACTGATGAAACCTTCAAGACCAGGGAGGCCATCCAGCATGAGTCTGGCAT | |
| CCTGGGCCCCCTGCTGTATGGGGAGGTGGGGGACACCCTGCTGATCATCTTCAAGAACCAGGCCAGCAGG | |
| CCCTACAACATCTACCCCCATGGCATCACTGATGTGAGGCCCCTGTACAGCAGGAGGCTGCCCAAGGGGG | |
| TGAAGCACCTGAAGGACTTCCCCATCCTGCCTGGGGAGATCTTCAAGTACAAGTGGACTGTGACTGTGGA | |
| GGATGGCCCCACCAAGTCTGACCCCAGGTGCCTGACCAGATACTACAGCAGCTTTGTGAACATGGAGAGG | |
| GACCTGGCCTCTGGCCTGATTGGCCCCCTGCTGATCTGCTACAAGGAGTCTGTGGACCAGAGGGGCAACC | |
| AGATCATGTCTGACAAGAGGAATGTGATCCTGTTCTCTGTGTTTGATGAGAACAGGAGCTGGTACCTGAC | |
| TGAGAACATCCAGAGGTTCCTGCCCAACCCTGCTGGGGTGCAGCTGGAGGACCCTGAGTTCCAGGCCAGC | |
| AACATCATGCACAGCATCAATGGCTATGTGTTTGACAGCCTGCAGCTGTCTGTGTGCCTGCATGAGGTGG | |
| CCTACTGGTACATCCTGAGCATTGGGGCCCAGACTGACTTCCTGTCTGTGTTCTTCTCTGGCTACACCTT | |
| CAAGCACAAGATGGTGTATGAGGACACCCTGACCCTGTTCCCCTTCTCTGGGGAGACTGTGTTCATGAGC | |
| ATGGAGAACCCTGGCCTGTGGATTCTGGGCTGCCACAACTCTGACTTCAGGAACAGGGGCATGACTGCCC | |
| TGCTGAAAGTCTCCAGCTGTGACAAGAACACTGGGGACTACTATGAGGACAGCTATGAGGACATCTCTGC | |
| CTACCTGCTGAGCAAGAACAATGCCATTGAGCCCAGGAGCTTCAGCCAGAATGCCACTAATGTGTCTAAC | |
| AACAGCAACACCAGCAATGACAGCAATGTGTCTCCCCCAGTGCTGAAGAGGCACCAGAGGGAGATCACCA | |
| GGACCACCCTGCAGTCTGACCAGGAGGAGATTGACTATGATGACACCATCTCTGTGGAGATGAAGAAGGA | |
| GGACTTTGACATCTACGACGAGGACGAGAACCAGAGCCCCAGGAGCTTCCAGAAGAAGACCAGGCACTAC | |
| TTCATTGCTGCTGTGGAGAGGCTGTGGGACTATGGCATGAGCAGCAGCCCCCATGTGCTGAGGAACAGGG | |
| CCCAGTCTGGCTCTGTGCCCCAGTTCAAGAAGGTGGTGTTCCAGGAGTTCACTGATGGCAGCTTCACCCA | |
| GCCCCTGTACAGAGGGGAGCTGAATGAGCACCTGGGCCTGCTGGGCCCCTACATCAGGGCTGAGGTGGAG | |
| GACAACATCATGGTGACCTTCAGGAACCAGGCCAGCAGGCCCTACAGCTTCTACAGCAGCCTGATCAGCT | |
| ATGAGGAGGACCAGAGGCAGGGGGCTGAGCCCAGGAAGAACTTTGTGAAGCCCAATGAAACCAAGACCTA | |
| CTTCTGGAAGGTGCAGCACCACATGGCCCCCACCAAGGATGAGTTTGACTGCAAGGCCTGGGCCTACTTC | |
| TCTGATGTGGACCTGGAGAAGGATGTGCACTCTGGCCTGATTGGCCCCCTGCTGGTGTGCCACACCAACA | |
| CCCTGAACCCTGCCCATGGCAGGCAGGTGACTGTGCAGGAGTTTGCCCTGTTCTTCACCATCTTTGATGA | |
| AACCAAGAGCTGGTACTTCACTGAGAACATGGAGAGGAACTGCAGGGCCCCCTGCAACATCCAGATGGAG | |
| GACCCCACCTTCAAGGAGAACTACAGGTTCCATGCCATCAATGGCTACATCATGGACACCCTGCCTGGCC | |
| TGGTGATGGCCCAGGACCAGAGGATCAGGTGGTACCTGCTGAGCATGGGCAGCAATGAGAACATCCACAG | |
| CATCCACTTCTCTGGCCATGTGTTCACTGTGAGGAAGAAGGAGGAGTACAAGATGGCCCTGTACAACCTG | |
| TACCCTGGGGTGTTTGAGACTGTGGAGATGCTGCCCAGCAAGGCTGGCATCTGGAGGGTGGAGTGCCTGA | |
| TTGGGGAGCACCTGCATGCTGGCATGAGCACCCTGTTCCTGGTGTACAGCAACAAGTGCCAGACCCCCCT | |
| GGGCATGGCCTCTGGCCACATCAGGGACTTCCAGATCACTGCCTCTGGCCAGTATGGCCAGTGGGCCCCC | |
| AAGCTGGCCAGGCTGCACTACTCTGGCAGCATCAATGCCTGGAGCACCAAGGAGCCCTTCAGCTGGATCA | |
| AGGTGGACCTGCTGGCCCCCATGATCATCCATGGCATCAAGACCCAGGGGGCCAGGCAGAAGTTCAGCAG | |
| CCTGTACATCAGCCAGTTCATCATCATGTACAGCCTGGATGGCAAGAAGTGGCAGACCTACAGGGGCAAC | |
| AGCACTGGCACCCTGATGGTGTTCTTTGGCAATGTGGACAGCTCTGGCATCAAGCACAACATCTTCAACC | |
| CCCCCATCATTGCCAGATACATCAGGCTGCACCCCACCCACTACAGCATCAGGAGCACCCTGAGGATGGA | |
| GCTGATGGGCTGTGACCTGAACAGCTGCAGCATGCCCCTGGGCATGGAGAGCAAGGCCATCTCTGATGCC | |
| CAGATCACTGCCAGCAGCTACTTCACCAACATGTTTGCCACCTGGAGCCCCAGCAAGGCCAGGCTGCACC | |
| TGCAGGGCAGGAGCAATGCCTGGAGGCCCCAGGTCAACAACCCCAAGGAGTGGCTGCAGGTGGACTTCCA | |
| GAAGACCATGAAGGTGACTGGGGTGACCACCCAGGGGGTGAAGAGCCTGCTGACCAGCATGTATGTGAAG | |
| GAGTTCCTGATCAGCAGCAGCCAGGATGGCCACCAGTGGACCCTGTTCTTCCAGAATGGCAAGGTGAAGG | |
| TGTTCCAGGGCAACCAGGACAGCTTCACCCCTGTGGTGAACAGCCTGGACCCCCCCCTGCTGACCAGATA | |
| CCTGAGGATTCACCCCCAGAGCTGGGTGCACCAGATTGCCCTGAGGATGGAGGTGCTGGGCTGTGAGGCC | |
| CAGGACCTGTACTGA | |
| Nucleic acid sequence encoding signal peptide is underlined. | |
| Complementary strand to the exemplified FVIII transgene (N6) | |
| SEQ ID NO: 27 | |
| TACGTCTAACTCGACTCGTGGACGAAGAAGGACACGGACGACTCCAAGACGAAGAGACGGTGGTCCTCTA | |
| TGATGGACCCCCGACACCTCGACTCGACCCTGATGTACGTCAGACTGGACCCCCTCGACGGACACCTACG | |
| GTCCAAGGGGGGGTCTCACGGGTTCTCGAAGGGGAAGTTGTGGAGACACCACATGTTCTTCTGGGACAAA | |
| CACCTCAAGTGACTGGTGGACAAGTTGTAACGGTTCGGGTCCGGGGGGACCTACCCGGACGACCCGGGGT | |
| GGTAGGTCCGACTCCACATACTGTGACACCACTAGTGGGACTTCTTGTACCGGTCGGTGGGACACTCGGA | |
| CGTACGACACCCCCACTCGATGACCTTCCGGAGACTCCCCCGACTCATACTACTGGTCTGGTCGGTCTCC | |
| CTCTTCCTCCTACTGTTCCACAAGGGACCCCCGTCGGTGTGGATACACACCGTCCACGACTTCCTCTTAC | |
| CGGGGTACCGGAGACTGGGGGACACGGACTGGATGTCGATGGACTCGGTACACCTGGACCACTTCCTGGA | |
| CTTGAGACCGGACTAACCCCGGGACGACCACACGTCCCTCCCGTCGGACCGGTTCCTCTTCTGGGTCTGG | |
| GACGTGTTCAAGTAGGACGACAAACGACACAAACTACTCCCGTTCTCGACCGTGAGACTTTGGTTCTTGT | |
| CGGACTACGTCCTGTCCCTACGACGGAGACGGTCCCGGACCGGGTTCTACGTGTGACACTTACCGATACA | |
| CTTGTCCTCGGACGGACCGGACTAACCGACGGTGTCCTTCAGACACATGACCGTACACTAACCGTACCCG | |
| TGGTGGGGACTCCACGTGTCGTAGAAGGACCTCCCGGTGTGGAAGGACCAGTCCTTGGTGTCCGTCCGGT | |
| CGGACCTCTAGTCGGGGTAGTGGAAGGACTGACGGGTCTGGGACGACTACCTGGACCCGGTCAAGGACGA | |
| CAAGACGGTGTAGTCGTCGGTGGTCGTACTACCGTACCTCCGGATACACTTCCACCTGTCGACGGGACTC | |
| CTCGGGGTCGACTCCTACTTCTTGTTACTCCTCCGACTCCTGATACTACTACTGGACTGACTGAGACTCT | |
| ACCTACACCACTCCAAACTACTACTGTTGTCGGGGTCGAAGTAGGTCTAGTCCAGACACCGGTTCTTCGT | |
| GGGGTTCTGGACCCACGTGATGTAACGACGACTCCTCCTCCTGACCCTGATACGGGGGGACCACGACCGG | |
| GGACTACTGTCCTCGATGTTCTCGGTCATGGACTTGTTACCGGGGGTCTCCTAACCGTCCTTCATGTTCT | |
| TCCAGTCCAAGTACCGGATGTGACTACTTTGGAAGTTCTGGTCCCTCCGGTAGGTCGTACTCAGACCGTA | |
| GGACCCGGGGGACGACATACCCCTCCACCCCCTGTGGGACGACTAGTAGAAGTTCTTGGTCCGGTCGTCC | |
| GGGATGTTGTAGATGGGGGTACCGTAGTGACTACACTCCGGGGACATGTCGTCCTCCGACGGGTTCCCCC | |
| ACTTCGTGGACTTCCTGAAGGGGTAGGACGGACCCCTCTAGAAGTTCATGTTCACCTGACACTGACACCT | |
| CCTACCGGGGTGGTTCAGACTGGGGTCCACGGACTGGTCTATGATGTCGTCGAAACACTTGTACCTCTCC | |
| CTGGACCGGAGACCGGACTAACCGGGGGACGACTAGACGATGTTCCTCAGACACCTGGTCTCCCCGTTGG | |
| TCTAGTACAGACTGTTCTCCTTACACTAGGACAAGAGACACAAACTACTCTTGTCCTCGACCATGGACTG | |
| ACTCTTGTAGGTCTCCAAGGACGGGTTGGGACGACCCCACGTCGACCTCCTGGGACTCAAGGTCCGGTCG | |
| TTGTAGTACGTGTCGTAGTTACCGATACACAAACTGTCGGACGTCGACAGACACACGGACGTACTCCACC | |
| GGATGACCATGTAGGACTCGTAACCCCGGGTCTGACTGAAGGACAGACACAAGAAGAGACCGATGTGGAA | |
| GTTCGTGTTCTACCACATACTCCTGTGGGACTGGGACAAGGGGAAGAGACCCCTCTGACACAAGTACTCG | |
| TACCTCTTGGGACCGGACACCTAAGACCCGACGGTGTTGAGACTGAAGTCCTTGTCCCCGTACTGACGGG | |
| ACGACTTTCAGAGGTCGACACTGTTCTTGTGACCCCTGATGATACTCCTGTCGATACTCCTGTAGAGACG | |
| GATGGACGACTCGTTCTTGTTACGGTAACTCGGGTCCTCGAAGTCGGTCTTGTCGTCCGTGGGGTCGTGG | |
| TCCGTCTTCGTCAAGTTACGGTGGTGGTAGGGACTCTTACTGTATCTCTTCTGTCTGGGTACCAAACGGG | |
| TGGCCTGGGGGTACGGGTTCTAGGTCTTACACTCGTCGAGACTGGACGACTACGACGACTCCGTCTCGGG | |
| GTGGGGGGTACCGGACTCGGACAGACTGGACGTCCTCCGGTTCATACTTTGGAAGAGACTACTGGGGTCG | |
| GGACCCCGGTAACTGTCGTTGTTGTCGGACAGACTCTACTGGGTGAAGTCCGGGGTCGACGTGGTGAGAC | |
| CCCTGTACCACAAGTGGGGACTCAGACCGGACGTCGACTCCGACTTACTCTTCGACCCGTGGTGACGACG | |
| GTGACTCGACTTCTTCGACCTGAAGTTTCAGAGGTCGTGGTCGTTGTTGGACTAGTCGTGGTAGGGGAGA | |
| CTGTTGGACCGACGACCGTGACTGTTGTGGTCGTCGGACCCGGGGGGGTCGTACGGACACGTGATACTGT | |
| CGGTCGACCTGTGGTGGGACAAACCGTTCTTCTCGTCGGGGGACTGACTCAGACCCCCGGGGGACTCGGA | |
| CAGACTCCTCTTGTTACTGTCGTTCGACGACCTCAGACCGGACTACTTGTCGGTCCTCTCGTCGACCCCG | |
| TTCTTACACTCGTCGTCCCTCTAGTGGTCCTGGTGGGACGTCAGACTGGTCCTCCTCTAACTGATACTAC | |
| TGTGGTAGAGACACCTCTACTTCTTCCTCCTGAAACTGTAGATGCTGCTCCTGCTCTTGGTCTCGGGGTC | |
| CTCGAAGGTCTTCTTCTGGTCCGTGATGAAGTAACGACGACACCTCTCCGACACCCTGATACCGTACTCG | |
| TCGTCGGGGGTACACGACTCCTTGTCCCGGGTCAGACCGAGACACGGGGTCAAGTTCTTCCACCACAAGG | |
| TCCTCAAGTGACTACCGTCGAAGTGGGTCGGGGACATGTCTCCCCTCGACTTACTCGTGGACCCGGACGA | |
| CCCGGGGATGTAGTCCCGACTCCACCTCCTGTTGTAGTACCACTGGAAGTCCTTGGTCCGGTCGTCCGGG | |
| ATGTCGAAGATGTCGTCGGACTAGTCGATACTCCTCCTGGTCTCCGTCCCCCGACTCGGGTCCTTCTTGA | |
| AACACTTCGGGTTACTTTGGTTCTGGATGAAGACCTTCCACGTCGTGGTGTACCGGGGGTGGTTCCTACT | |
| CAAACTGACGTTCCGGACCCGGATGAAGAGACTACACCTGGACCTCTTCCTACACGTGAGACCGGACTAA | |
| CCGGGGGACGACCACACGGTGTGGTTGTGGGACTTGGGACGGGTACCGTCCGTCCACTGACACGTCCTCA | |
| AACGGGACAAGAAGTGGTAGAAACTACTTTGGTTCTCGACCATGAAGTGACTCTTGTACCTCTCCTTGAC | |
| GTCCCGGGGGACGTTGTAGGTCTACCTCCTGGGGTGGAAGTTCCTCTTGATGTCCAAGGTACGGTAGTTA | |
| CCGATGTAGTACCTGTGGGACGGACCGGACCACTACCGGGTCCTGGTCTCCTAGTCCACCATGGACGACT | |
| CGTACCCGTCGTTACTCTTGTAGGTGTCGTAGGTGAAGAGACCGGTACACAAGTGACACTCCTTCTTCCT | |
| CCTCATGTTCTACCGGGACATGTTGGACATGGGACCCCACAAACTCTGACACCTCTACGACGGGTCGTTC | |
| CGACCGTAGACCTCCCACCTCACGGACTAACCCCTCGTGGACGTACGACCGTACTCGTGGGACAAGGACC | |
| ACATGTCGTTGTTCACGGTCTGGGGGGACCCGTACCGGAGACCGGTGTAGTCCCTGAAGGTCTAGTGACG | |
| GAGACCGGTCATACCGGTCACCCGGGGGTTCGACCGGTCCGACGTGATGAGACCGTCGTAGTTACGGACC | |
| TCGTGGTTCCTCGGGAAGTCGACCTAGTTCCACCTGGACGACCGGGGGTACTAGTAGGTACCGTAGTTCT | |
| GGGTCCCCCGGTCCGTCTTCAAGTCGTCGGACATGTAGTCGGTCAAGTAGTAGTACATGTCGGACCTACC | |
| GTTCTTCACCGTCTGGATGTCCCCGTTGTCGTGACCGTGGGACTACCACAAGAAACCGTTACACCTGTCG | |
| AGACCGTAGTTCGTGTTGTAGAAGTTGGGGGGGTAGTAACGGTCTATGTAGTCCGACGTGGGGTGGGTGA | |
| TGTCGTAGTCCTCGTGGGACTCCTACCTCGACTACCCGACACTGGACTTGTCGACGTCGTACGGGGACCC | |
| GTACCTCTCGTTCCGGTAGAGACTACGGGTCTAGTGACGGTCGTCGATGAAGTGGTTGTACAAACGGTGG | |
| ACCTCGGGGTCGTTCCGGTCCGACGTGGACGTCCCGTCCTCGTTACGGACCTCCGGGGTCCAGTTGTTGG | |
| GGTTCCTCACCGACGTCCACCTGAAGGTCTTCTGGTACTTCCACTGACCCCACTGGTGGGTCCCCCACTT | |
| CTCGGACGACTGGTCGTACATACACTTCCTCAAGGACTAGTCGTCGTCGGTCCTACCGGTGGTCACCTGG | |
| GACAAGAAGGTCTTACCGTTCCACTTCCACAAGGTCCCGTTGGTCCTGTCGAAGTGGGGACACCACTTGT | |
| CGGACCTGGGGGGGGACGACTGGTCTATGGACTCCTAAGTGGGGGTCTCGACCCACGTGGTCTAACGGGA | |
| CTCCTACCTCCACGACCCGACACTCCGGGTCCTGGACATGACT | |
| Complementary strand to the exemplified FVIII transgene (V3) | |
| SEQ ID NO: 28 | |
| TACGTCTAACTCGACTCGTGGACGAAGAAGGACACGGACGACTCCAAGACGAAGAGACGGTGGTCCTCTA | |
| TGATGGACCCCCGACACCTCGACTCGACCCTGATGTACGTCAGACTGGACCCCCTCGACGGACACCTACG | |
| GTCCAAGGGGGGGTCTCACGGGTTCTCGAAGGGGAAGTTGTGGAGACACCACATGTTCTTCTGGGACAAA | |
| CACCTCAAGTGACTGGTGGACAAGTTGTAACGGTTCGGGTCCGGGGGGACCTACCCGGACGACCCGGGGT | |
| GGTAGGTCCGACTCCACATACTGTGACACCACTAGTGGGACTTCTTGTACCGGTCGGTGGGACACTCGGA | |
| CGTACGACACCCCCACTCGATGACCTTCCGGAGACTCCCCCGACTCATACTACTGGTCTGGTCGGTCTCC | |
| CTCTTCCTCCTACTGTTCCACAAGGGACCCCCGTCGGTGTGGATACACACCGTCCACGACTTCCTCTTAC | |
| CGGGGTACCGGAGACTGGGGGACACGGACTGGATGTCGATGGACTCGGTACACCTGGACCACTTCCTGGA | |
| CTTGAGACCGGACTAACCCCGGGACGACCACACGTCCCTCCCGTCGGACCGGTTCCTCTTCTGGGTCTGG | |
| GACGTGTTCAAGTAGGACGACAAACGACACAAACTACTCCCGTTCTCGACCGTGAGACTTTGGTTCTTGT | |
| CGGACTACGTCCTGTCCCTACGACGGAGACGGTCCCGGACCGGGTTCTACGTGTGACACTTACCGATACA | |
| CTTGTCCTCGGACGGACCGGACTAACCGACGGTGTCCTTCAGACACATGACCGTACACTAACCGTACCCG | |
| TGGTGGGGACTCCACGTGTCGTAGAAGGACCTCCCGGTGTGGAAGGACCAGTCCTTGGTGTCCGTCCGGT | |
| CGGACCTCTAGTCGGGGTAGTGGAAGGACTGACGGGTCTGGGACGACTACCTGGACCCGGTCAAGGACGA | |
| CAAGACGGTGTAGTCGTCGGTGGTCGTACTACCGTACCTCCGGATACACTTCCACCTGTCGACGGGACTC | |
| CTCGGGGTCGACTCCTACTTCTTGTTACTCCTCCGACTCCTGATACTACTACTGGACTGACTGAGACTCT | |
| ACCTACACCACTCCAAACTACTACTGTTGTCGGGGTCGAAGTAGGTCTAGTCCAGACACCGGTTCTTCGT | |
| GGGGTTCTGGACCCACGTGATGTAACGACGACTCCTCCTCCTGACCCTGATACGGGGGGACCACGACCGG | |
| GGACTACTGTCCTCGATGTTCTCGGTCATGGACTTGTTACCGGGGGTCTCCTAACCGTCCTTCATGTTCT | |
| TCCAGTCCAAGTACCGGATGTGACTACTTTGGAAGTTCTGGTCCCTCCGGTAGGTCGTACTCAGACCGTA | |
| GGACCCGGGGGACGACATACCCCTCCACCCCCTGTGGGACGACTAGTAGAAGTTCTTGGTCCGGTCGTCC | |
| GGGATGTTGTAGATGGGGGTACCGTAGTGACTACACTCCGGGGACATGTCGTCCTCCGACGGGTTCCCCC | |
| ACTTCGTGGACTTCCTGAAGGGGTAGGACGGACCCCTCTAGAAGTTCATGTTCACCTGACACTGACACCT | |
| CCTACCGGGGTGGTTCAGACTGGGGTCCACGGACTGGTCTATGATGTCGTCGAAACACTTGTACCTCTCC | |
| CTGGACCGGAGACCGGACTAACCGGGGGACGACTAGACGATGTTCCTCAGACACCTGGTCTCCCCGTTGG | |
| TCTAGTACAGACTGTTCTCCTTACACTAGGACAAGAGACACAAACTACTCTTGTCCTCGACCATGGACTG | |
| ACTCTTGTAGGTCTCCAAGGACGGGTTGGGACGACCCCACGTCGACCTCCTGGGACTCAAGGTCCGGTCG | |
| TTGTAGTACGTGTCGTAGTTACCGATACACAAACTGTCGGACGTCGACAGACACACGGACGTACTCCACC | |
| GGATGACCATGTAGGACTCGTAACCCCGGGTCTGACTGAAGGACAGACACAAGAAGAGACCGATGTGGAA | |
| GTTCGTGTTCTACCACATACTCCTGTGGGACTGGGACAAGGGGAAGAGACCCCTCTGACACAAGTACTCG | |
| TACCTCTTGGGACCGGACACCTAAGACCCGACGGTGTTGAGACTGAAGTCCTTGTCCCCGTACTGACGGG | |
| ACGACTTTCAGAGGTCGACACTGTTCTTGTGACCCCTGATGATACTCCTGTCGATACTCCTGTAGAGACG | |
| GATGGACGACTCGTTCTTGTTACGGTAACTCGGGTCCTCGAAGTCGGTCTTACGGTGATTACACAGATTG | |
| TTGTCGTTGTGGTCGTTACTGTCGTTACACAGAGGGGGTCACGACTTCTCCGTGGTCTCCCTCTAGTGGT | |
| CCTGGTGGGACGTCAGACTGGTCCTCCTCTAACTGATACTACTGTGGTAGAGACACCTCTACTTCTTCCT | |
| CCTGAAACTGTAGATGCTGCTCCTGCTCTTGGTCTCGGGGTCCTCGAAGGTCTTCTTCTGGTCCGTGATG | |
| AAGTAACGACGACACCTCTCCGACACCCTGATACCGTACTCGTCGTCGGGGGTACACGACTCCTTGTCCC | |
| GGGTCAGACCGAGACACGGGGTCAAGTTCTTCCACCACAAGGTCCTCAAGTGACTACCGTCGAAGTGGGT | |
| CGGGGACATGTCTCCCCTCGACTTACTCGTGGACCCGGACGACCCGGGGATGTAGTCCCGACTCCACCTC | |
| CTGTTGTAGTACCACTGGAAGTCCTTGGTCCGGTCGTCCGGGATGTCGAAGATGTCGTCGGACTAGTCGA | |
| TACTCCTCCTGGTCTCCGTCCCCCGACTCGGGTCCTTCTTGAAACACTTCGGGTTACTTTGGTTCTGGAT | |
| GAAGACCTTCCACGTCGTGGTGTACCGGGGGTGGTTCCTACTCAAACTGACGTTCCGGACCCGGATGAAG | |
| AGACTACACCTGGACCTCTTCCTACACGTGAGACCGGACTAACCGGGGGACGACCACACGGTGTGGTTGT | |
| GGGACTTGGGACGGGTACCGTCCGTCCACTGACACGTCCTCAAACGGGACAAGAAGTGGTAGAAACTACT | |
| TTGGTTCTCGACCATGAAGTGACTCTTGTACCTCTCCTTGACGTCCCGGGGGACGTTGTAGGTCTACCTC | |
| CTGGGGTGGAAGTTCCTCTTGATGTCCAAGGTACGGTAGTTACCGATGTAGTACCTGTGGGACGGACCGG | |
| ACCACTACCGGGTCCTGGTCTCCTAGTCCACCATGGACGACTCGTACCCGTCGTTACTCTTGTAGGTGTC | |
| GTAGGTGAAGAGACCGGTACACAAGTGACACTCCTTCTTCCTCCTCATGTTCTACCGGGACATGTTGGAC | |
| ATGGGACCCCACAAACTCTGACACCTCTACGACGGGTCGTTCCGACCGTAGACCTCCCACCTCACGGACT | |
| AACCCCTCGTGGACGTACGACCGTACTCGTGGGACAAGGACCACATGTCGTTGTTCACGGTCTGGGGGGA | |
| CCCGTACCGGAGACCGGTGTAGTCCCTGAAGGTCTAGTGACGGAGACCGGTCATACCGGTCACCCGGGGG | |
| TTCGACCGGTCCGACGTGATGAGACCGTCGTAGTTACGGACCTCGTGGTTCCTCGGGAAGTCGACCTAGT | |
| TCCACCTGGACGACCGGGGGTACTAGTAGGTACCGTAGTTCTGGGTCCCCCGGTCCGTCTTCAAGTCGTC | |
| GGACATGTAGTCGGTCAAGTAGTAGTACATGTCGGACCTACCGTTCTTCACCGTCTGGATGTCCCCGTTG | |
| TCGTGACCGTGGGACTACCACAAGAAACCGTTACACCTGTCGAGACCGTAGTTCGTGTTGTAGAAGTTGG | |
| GGGGGTAGTAACGGTCTATGTAGTCCGACGTGGGGTGGGTGATGTCGTAGTCCTCGTGGGACTCCTACCT | |
| CGACTACCCGACACTGGACTTGTCGACGTCGTACGGGGACCCGTACCTCTCGTTCCGGTAGAGACTACGG | |
| GTCTAGTGACGGTCGTCGATGAAGTGGTTGTACAAACGGTGGACCTCGGGGTCGTTCCGGTCCGACGTGG | |
| ACGTCCCGTCCTCGTTACGGACCTCCGGGGTCCAGTTGTTGGGGTTCCTCACCGACGTCCACCTGAAGGT | |
| CTTCTGGTACTTCCACTGACCCCACTGGTGGGTCCCCCACTTCTCGGACGACTGGTCGTACATACACTTC | |
| CTCAAGGACTAGTCGTCGTCGGTCCTACCGGTGGTCACCTGGGACAAGAAGGTCTTACCGTTCCACTTCC | |
| ACAAGGTCCCGTTGGTCCTGTCGAAGTGGGGACACCACTTGTCGGACCTGGGGGGGGACGACTGGTCTAT | |
| GGACTCCTAAGTGGGGGTCTCGACCCACGTGGTCTAACGGGACTCCTACCTCCACGACCCGACACTCCGG | |
| GTCCTGGACATGACT | |
| Exemplified FVIII polypeptide (N6) | |
| SEQ ID NO: 29 | |
| MQIELSTCFFLCLLRFCFSATRRYYLGAVELSWDYMQSDLGELPVDARFPPRVPKSFPFNTSVVYKKTLFV | |
| EFTDHLFNIAKPRPPWMGLLGPTIQAEVYDTVVITLKNMASHPVSLHAVGVSYWKASEGAEYDDQTSQREK | |
| EDDKVFPGGSHTYVWQVLKENGPMASDPLCLTYSYLSHVDLVKDLNSGLIGALLVCREGSLAKEKTQTLHK | |
| FILLFAVEDEGKSWHSETKNSLMQDRDAASARAWPKMHTVNGYVNRSLPGLIGCHRKSVYWHVIGMGTTPE | |
| VHSIFLEGHTFLVRNHRQASLEISPITFLTAQTLLMDLGQFLLFCHISSHQHDGMEAYVKVDSCPEEPQLR | |
| MKNNEEAEDYDDDLTDSEMDVVRFDDDNSPSFIQIRSVAKKHPKTWVHYIAAEEEDWDYAPLVLAPDDRSY | |
| KSQYLNNGPQRIGRKYKKVRFMAYTDETFKTREAIQHESGILGPLLYGEVGDTLLIIFKNQASRPYNIYPH | |
| GITDVRPLYSRRLPKGVKHLKDFPILPGEIFKYKWTVTVEDGPTKSDPRCLTRYYSSFVNMERDLASGLIG | |
| PLLICYKESVDQRGNQIMSDKRNVILFSVEDENRSWYLTENIQRFLPNPAGVQLEDPEFQASNIMHSINGY | |
| VFDSLQLSVCLHEVAYWYILSIGAQTDFLSVFFSGYTFKHKMVYEDTLTLFPFSGETVFMSMENPGLWILG | |
| CHNSDFRNRGMTALLKVSSCDKNTGDYYEDSYEDISAYLLSKNNAIEPRSFSQNSRHPSTRQKQFNATTIP | |
| ENDIEKTDPWFAHRTPMPKIQNVSSSDLLMLLRQSPTPHGLSLSDLQEAKYETFSDDPSPGAIDSNNSLSE | |
| MTHFRPQLHHSGDMVFTPESGLQLRLNEKLGTTAATELKKLDFKVSSTSNNLISTIPSDNLAAGTDNTSSL | |
| GPPSMPVHYDSQLDTTLFGKKSSPLTESGGPLSLSEENNDSKLLESGLMNSQESSWGKNVSSREITRTTLQ | |
| SDQEEIDYDDTISVEMKKEDEDIYDEDENQSPRSFQKKTRHYFIAAVERLWDYGMSSSPHVLRNRAQSGSV | |
| PQFKKVVFQEFTDGSFTQPLYRGELNEHLGLLGPYIRAEVEDNIMVTFRNQASRPYSFYSSLISYEEDQRQ | |
| GAEPRKNFVKPNETKTYFWKVQHHMAPTKDEFDCKAWAYFSDVDLEKDVHSGLIGPLLVCHTNTLNPAHGR | |
| QVTVQEFALFFTIFDETKSWYFTENMERNCRAPCNIQMEDPTFKENYRFHAINGYIMDTLPGLVMAQDQRI | |
| RWYLLSMGSNENIHSIHFSGHVFTVRKKEEYKMALYNLYPGVFETVEMLPSKAGIWRVECLIGEHLHAGMS | |
| TLFLVYSNKCQTPLGMASGHIRDFQITASGQYGQWAPKLARLHYSGSINAWSTKEPFSWIKVDLLAPMIIH | |
| GIKTQGARQKFSSLYISQFIIMYSLDGKKWQTYRGNSTGTLMVFFGNVDSSGIKHNIFNPPIIARYIRLHP | |
| THYSIRSTLRMELMGCDLNSCSMPLGMESKAISDAQITASSYFTNMFATWSPSKARLHLQGRSNAWRPQVN | |
| NPKEWLQVDFQKTMKVTGVTTQGVKSLLTSMYVKEFLISSSQDGHQWTLFFQNGKVKVFQGNQDSFTPVVN | |
| SLDPPLLTRYLRIHPQSWVHQIALRMEVLGCEAQDLY | |
| Signal peptide is underlined. | |
| Exemplified FVIII polypeptide (V3) | |
| SEQ ID NO: 30 | |
| MQIELSTCFFLCLLRFCFSATRRYYLGAVELSWDYMQSDLGELPVDARFPPRVPKSFPFNTSVVYKKTLF | |
| VEFTDHLFNIAKPRPPWMGLLGPTIQAEVYDTVVITLKNMASHPVSLHAVGVSYWKASEGAEYDDQTSQR | |
| EKEDDKVFPGGSHTYVWQVLKENGPMASDPLCLTYSYLSHVDLVKDLNSGLIGALLVCREGSLAKEKTQT | |
| LHKFILLFAVFDEGKSWHSETKNSLMQDRDAASARAWPKMHTVNGYVNRSLPGLIGCHRKSVYWHVIGMG | |
| TTPEVHSIFLEGHTFLVRNHRQASLEISPITFLTAQTLLMDLGQFLLFCHISSHQHDGMEAYVKVDSCPE | |
| EPQLRMKNNEEAEDYDDDLTDSEMDVVRFDDDNSPSFIQIRSVAKKHPKTWVHYIAAEEEDWDYAPLVLA | |
| PDDRSYKSQYLNNGPQRIGRKYKKVRFMAYTDETFKTREAIQHESGILGPLLYGEVGDTLLIIFKNQASR | |
| PYNIYPHGITDVRPLYSRRLPKGVKHLKDFPILPGEIFKYKWTVTVEDGPTKSDPRCLTRYYSSFVNMER | |
| DLASGLIGPLLICYKESVDQRGNQIMSDKRNVILFSVEDENRSWYLTENIQRFLPNPAGVQLEDPEFQAS | |
| NIMHSINGYVFDSLQLSVCLHEVAYWYILSIGAQTDFLSVFFSGYTFKHKMVYEDTLTLFPFSGETVFMS | |
| MENPGLWILGCHNSDFRNRGMTALLKVSSCDKNTGDYYEDSYEDISAYLLSKNNAIEPRSFSQNATNVSN | |
| NSNTSNDSNVSPPVLKRHQREITRTTLQSDQEEIDYDDTISVEMKKEDEDIYDEDENQSPRSFQKKTRHY | |
| FIAAVERLWDYGMSSSPHVLRNRAQSGSVPQFKKVVFQEFTDGSFTQPLYRGELNEHLGLLGPYIRAEVE | |
| DNIMVTFRNQASRPYSFYSSLISYEEDQRQGAEPRKNFVKPNETKTYFWKVQHHMAPTKDEFDCKAWAYF | |
| SDVDLEKDVHSGLIGPLLVCHTNTLNPAHGRQVTVQEFALFFTIFDETKSWYFTENMERNCRAPCNIQME | |
| DPTFKENYRFHAINGYIMDTLPGLVMAQDQRIRWYLLSMGSNENIHSIHFSGHVFTVRKKEEYKMALYNL | |
| YPGVFETVEMLPSKAGIWRVECLIGEHLHAGMSTLFLVYSNKCQTPLGMASGHIRDFQITASGQYGQWAP | |
| KLARLHYSGSINAWSTKEPFSWIKVDLLAPMIIHGIKTQGARQKFSSLYISQFIIMYSLDGKKWQTYRGN | |
| STGTLMVFFGNVDSSGIKHNIFNPPIIARYIRLHPTHYSIRSTLRMELMGCDLNSCSMPLGMESKAISDA | |
| QITASSYFTNMFATWSPSKARLHLQGRSNAWRPQVNNPKEWLQVDFQKTMKVTGVTTQGVKSLLTSMYVK | |
| EFLISSSQDGHQWTLFFQNGKVKVFQGNQDSFTPVVNSLDPPLLTRYLRIHPQSWVHQIALRMEVLGCEA | |
| QDLY | |
| Signal peptide is underlined. | |
| Exemplified Human DCN (Decorin) transgene | |
| SEQ ID NO: 31 | |
| ATGAAGGCCACTATCATCCTCCTTCTGCTTGCACAAGTTTCCTGGGCTGGACCGTTTCAACAGAGAGGCTTATTT | |
| GACTTTATGCTAGAAGATGAGGCTTCTGGGATAGGCCCAGAAGTTCCTGATGACCGCGACTTCGAGCCCTCCCTA | |
| GGCCCAGTGTGCCCCTTCCGCTGTCAATGCCATCTTCGAGTGGTCCAGTGTTCTGATTTGGGTCTGGACAAAGTG | |
| CCAAAGGATCTTCCCCCTGACACAACTCTGCTAGACCTGCAAAACAACAAAATAACCGAAATCAAAGATGGAGAC | |
| TTTAAGAACCTGAAGAACCTTCACGCATTGATTCTTGTCAACAATAAAATTAGCAAAGTTAGTCCTGGAGCATTT | |
| ACACCTTTGGTGAAGTTGGAACGACTTTATCTGTCCAAGAATCAGCTGAAGGAATTGCCAGAAAAAATGCCCAAA | |
| ACTCTTCAGGAGCTGCGTGCCCATGAGAATGAGATCACCAAAGTGCGAAAAGTTACTTTCAATGGACTGAACCAG | |
| ATGATTGTCATAGAACTGGGCACCAATCCGCTGAAGAGCTCAGGAATTGAAAATGGGGCTTTCCAGGGAATGAAG | |
| AAGCTCTCCTACATCCGCATTGCTGATACCAATATCACCAGCATTCCTCAAGGTCTTCCTCCTTCCCTTACGGAA | |
| TTACATCTTGATGGCAACAAAATCAGCAGAGTTGATGCAGCTAGCCTGAAAGGACTGAATAATTTGGCTAAGTTG | |
| GGATTGAGTTTCAACAGCATCTCTGCTGTTGACAATGGCTCTCTGGCCAACACGCCTCATCTGAGGGAGCTTCAC | |
| TTGGACAACAACAAGCTTACCAGAGTACCTGGTGGGCTGGCAGAGCATAAGTACATCCAGGTTGTCTACCTTCAT | |
| AACAACAATATCTCTGTAGTTGGATCAAGTGACTTCTGCCCACCTGGACACAACACCAAAAAGGCTTCTTATTCG | |
| GGTGTGAGTCTTTTCAGCAACCCGGTCCAGTACTGGGAGATACAGCCATCCACCTTCAGATGTGTCTACGTGCGC | |
| TCTGCCATTCAACTCGGAAACTATAAGTAA | |
| Nucleic acid sequence encoding signal peptide is underlined. | |
| Exemplified Human Decorin polypeptide | |
| SEQ ID NO: 32 | |
| MKATIILLLLAQVSWAGPFQQRGLFDFMLEDEASGIGPEVPDDRDFEPSLGPVCPFRCQCHLRVVQCSDLGLDKV | |
| PKDLPPDTTLLDLQNNKITEIKDGDFKNLKNLHALILVNNKISKVSPGAFTPLVKLERLYLSKNQLKELPEKMPK | |
| TLQELRAHENEITKVRKVTFNGLNQMIVIELGTNPLKSSGIENGAFQGMKKLSYIRIADTNITSIPQGLPPSLTE | |
| LHLDGNKISRVDAASLKGLNNLAKLGLSFNSISAVDNGSLANTPHLRELHLDNNKLTRVPGGLAEHKYIQVVYLH | |
| NNNISVVGSSDFCPPGHNTKKASYSGVSLFSNPVQYWEIQPSTFRCVYVRSAIQLGNYK | |
| Signal peptide is underlined. | |
| Exemplified Human TRIM72 transgene | |
| SEQ ID NO: 33 | |
| ATGTCGGCTGCGCCCGGCCTCCTGCACCAGGAGCTGTCCTGCCCGCTGTGCCTGCAGCTGTTCGACGCGCCCGTG | |
| ACAGCCGAGTGCGGCCACAGTTTCTGCCGCGCCTGCCTAGGCCGCGTGGCCGGGGAGCCGGCGGCGGATGGCACC | |
| GTTCTCTGCCCCTGCTGCCAGGCCCCCACGCGGCCGCAGGCACTCAGCACCAACCTGCAGCTGGCGCGCCTGGTG | |
| GAGGGGCTGGCCCAGGTGCCGCAGGGCCACTGCGAGGAGCACCTGGACCCGCTGAGCATCTACTGCGAGCAGGAC | |
| CGCGCGCTGGTGTGCGGAGTGTGCGCCTCACTCGGCTCGCACCGCGGTCATCGCCTCCTGCCTGCCGCCGAGGCC | |
| CACGCACGCCTCAAGACACAGCTGCCACAGCAGAAACTGCAGCTGCAGGAGGCATGCATGCGCAAGGAGAAGAGT | |
| GTGGCTGTGCTGGAGCATCAGCTGGTGGAGGTGGAGGAGACAGTGCGTCAGTTCCGGGGGGCCGTGGGGGAGCAG | |
| CTGGGCAAGATGCGGGTGTTCCTGGCTGCACTGGAGGGCTCCTTGGACCGCGAGGCAGAGCGTGTACGGGGTGAG | |
| GCAGGGGTCGCCTTGCGCCGGGAGCTGGGGAGCCTGAACTCTTACCTGGAGCAGCTGCGGCAGATGGAGAAGGTC | |
| CTGGAGGAGGTGGCGGACAAGCCGCAGACTGAGTTCCTCATGAAATACTGCCTGGTGACCAGCAGGCTGCAGAAG | |
| ATCCTGGCAGAGTCTCCCCCACCCGCCCGTCTGGACATCCAGCTGCCAATTATCTCAGATGACTTCAAATTCCAG | |
| GTGTGGAGGAAGATGTTCCGGGCTCTGATGCCAGCGCTGGAGGAGCTGACCTTTGACCCGAGCTCTGCGCACCCG | |
| AGCCTGGTGGTGTCTTCCTCTGGCCGCCGCGTGGAGTGCTCGGAGCAGAAGGCGCCGCCGGCCGGGGAGGACCCG | |
| CGCCAGTTCGACAAGGCGGTGGCGGTGGTGGCGCACCAGCAGCTCTCCGAGGGCGAGCACTACTGGGAGGTGGAT | |
| GTTGGCGACAAGCCGCGCTGGGCGCTGGGCGTGATCGCGGCCGAGGCCCCCCGCCGCGGGCGCCTGCACGCGGTG | |
| CCCTCGCAGGGCCTGTGGCTGCTGGGGCTGCGCGAGGGCAAGATCCTGGAGGCACACGTGGAGGCCAAGGAGCCG | |
| CGCGCTCTGCGCAGCCCCGAGAGGCGGCCCACGCGCATTGGCCTTTACCTGAGCTTCGGCGACGGCGTCCTCTCC | |
| TTCTACGATGCCAGCGACGCCGACGCGCTCGTGCCGCTTTTTGCCTTCCACGAGCGCCTGCCCAGGCCCGTGTAC | |
| CCCTTCTTCGACGTGTGCTGGCACGACAAGGGCAAGAATGCCCAGCCGCTGCTGCTCGTGGGTCCCGAAGGCGCC | |
| GAGGCCTGA | |
| Exemplified Human TRIM72 polypeptide | |
| SEQ ID NO: 34 | |
| MSAAPGLLHQELSCPLCLQLFDAPVTAECGHSFCRACLGRVAGEPAADGTVLCPCCQAPTRPQALSTNLQLARLV | |
| EGLAQVPQGHCEEHLDPLSIYCEQDRALVCGVCASLGSHRGHRLLPAAEAHARLKTQLPQQKLQLQEACMRKEKS | |
| VAVLEHQLVEVEETVRQFRGAVGEQLGKMRVFLAALEGSLDREAERVRGEAGVALRRELGSLNSYLEQLRQMEKV | |
| LEEVADKPQTEFLMKYCLVTSRLQKILAESPPPARLDIQLPIISDDFKFQVWRKMFRALMPALEELTFDPSSAHP | |
| SLVVSSSGRRVECSEQKAPPAGEDPRQFDKAVAVVAHQQLSEGEHYWEVDVGDKPRWALGVIAAEAPRRGRLHAV | |
| PSQGLWLLGLREGKILEAHVEAKEPRALRSPERRPTRIGLYLSFGDGVLSFYDASDADALVPLFAFHERLPRPVY | |
| PFFDVCWHDKGKNAQPLLLVGPEGAEA | |
| Exemplified Human ABACA3 (ABCA3) transgene | |
| SEQ ID NO: 35 | |
| ATGGCTGTGCTCAGGCAGCTGGCGCTCCTCCTCTGGAAGAACTACACCCTGCAGAAGCGGAAGGTCCTGGTGACG | |
| GTCCTGGAACTCTTCCTGCCATTGCTGTTTTCTGGGATCCTCATCTGGCTCCGCTTGAAGATTCAGTCGGAAAAT | |
| GTGCCCAACGCCACCATCTACCCGGGCCAGTCCATCCAGGAGCTGCCTCTGTTCTTCACCTTCCCTCCGCCAGGA | |
| GACACCTGGGAGCTTGCCTACATCCCTTCTCACAGTGACGCTGCCAAGACCGTCACTGAGACAGTGCGCAGGGCA | |
| CTTGTGATCAACATGCGAGTGCGCGGCTTTCCCTCCGAGAAGGACTTTGAGGACTACATTAGGTACGACAACTGC | |
| TCGTCCAGCGTGCTGGCCGCCGTGGTCTTCGAGCACCCCTTCAACCACAGCAAGGAGCCCCTGCCGCTGGCGGTG | |
| AAATATCACCTACGGTTCAGTTACACACGGAGAAATTACATGTGGACCCAAACAGGCTCCTTTTTCCTGAAAGAG | |
| ACAGAAGGCTGGCACACTACTTCCCTTTTCCCGCTTTTCCCAAACCCAGGACCAAGGGAACCTACATCCCCTGAT | |
| GGCGGAGAACCTGGGTACATCCGGGAAGGCTTCCTGGCCGTGCAGCATGCTGTGGACCGGGCCATCATGGAGTAC | |
| CATGCCGATGCCGCCACACGCCAGCTGTTCCAGAGACTGACGGTGACCATCAAGAGGTTCCCGTACCCGCCGTTC | |
| ATCGCAGACCCCTTCCTCGTGGCCATCCAGTACCAGCTGCCCCTGCTGCTGCTGCTCAGCTTCACCTACACCGCG | |
| CTCACCATTGCCCGTGCTGTCGTGCAGGAGAAGGAAAGGAGGCTGAAGGAGTACATGCGCATGATGGGGCTCAGC | |
| AGCTGGCTGCACTGGAGTGCCTGGTTCCTCTTGTTCTTCCTCTTCCTCCTCATCGCCGCCTCCTTCATGACCCTG | |
| CTCTTCTGTGTCAAGGTGAAGCCAAATGTAGCCGTGCTGTCCCGCAGCGACCCCTCCCTGGTGCTCGCCTTCCTG | |
| CTGTGCTTCGCCATCTCTACCATCTCCTTCAGCTTCATGGTCAGCACCTTCTTCAGCAAAGCCAACATGGCAGCA | |
| GCCTTCGGAGGCTTCCTCTACTTCTTCACCTACATCCCCTACTTCTTCGTGGCCCCTCGGTACAACTGGATGACT | |
| CTGAGCCAGAAGCTCTGCTCCTGCCTCCTGTCTAATGTCGCCATGGCAATGGGAGCCCAGCTCATTGGGAAATTT | |
| GAGGCGAAAGGCATGGGCATCCAGTGGCGAGACCTCCTGAGTCCCGTCAACGTGGACGACGACTTCTGCTTCGGG | |
| CAGGTGCTGGGGATGCTGCTGCTGGACTCTGTGCTCTATGGCCTGGTGACCTGGTACATGGAGGCCGTCTTCCCA | |
| GGGCAGTTCGGCGTGCCTCAGCCCTGGTACTTCTTCATCATGCCCTCCTATTGGTGTGGGAAGCCAAGGGCGGTT | |
| GCAGGGAAGGAGGAAGAAGACAGTGACCCCGAGAAAGCACTCAGAAACGAGTACTTTGAAGCCGAGCCAGAGGAC | |
| CTGGTGGCGGGGATCAAGATCAAGCACCTGTCCAAGGTGTTCAGGGTGGGAAATAAGGACAGGGCGGCCGTCAGA | |
| GACCTGAACCTCAACCTGTACGAGGGACAGATCACCGTCCTGCTGGGCCACAACGGTGCCGGGAAGACCACCACC | |
| CTCTCCATGCTCACAGGTCTCTTTCCCCCCACCAGTGGACGGGCATACATCAGCGGGTATGAAATTTCCCAGGAC | |
| ATGGTTCAGATCCGGAAGAGCCTGGGCCTGTGCCCGCAGCACGACATCCTGTTTGACAACTTGACAGTCGCAGAG | |
| CACCTTTATTTCTACGCCCAGCTGAAGGGCCTGTCACGTCAGAAGTGCCCTGAAGAAGTCAAGCAGATGCTGCAC | |
| ATCATCGGCCTGGAGGACAAGTGGAACTCACGGAGCCGCTTCCTGAGCGGGGGCATGAGGCGCAAGCTCTCCATC | |
| GGCATCGCCCTCATCGCAGGCTCCAAGGTGCTGATACTGGACGAGCCCACCTCGGGCATGGACGCCATCTCCAGG | |
| AGGGCCATCTGGGATCTTCTTCAGCGGCAGAAAAGTGACCGCACCATCGTGCTGACCACCCACTTCATGGACGAG | |
| GCTGACCTGCTGGGAGACCGCATCGCCATCATGGCCAAGGGGGAGCTGCAGTGCTGCGGGTCCTCGCTGTTCCTC | |
| AAGCAGAAATACGGTGCCGGCTATCACATGACGCTGGTGAAGGAGCCGCACTGCAACCCGGAAGACATCTCCCAG | |
| CTGGTCCACCACCACGTGCCCAACGCCACGCTGGAGAGCAGCGCTGGGGCCGAGCTGTCTTTCATCCTTCCCAGA | |
| GAGAGCACGCACAGGTTTGAAGGTCTCTTTGCTAAACTGGAGAAGAAGCAGAAAGAGCTGGGCATTGCCAGCTTT | |
| GGGGCATCCATCACCACCATGGAGGAAGTCTTCCTTCGGGTCGGGAAGCTGGTGGACAGCAGTATGGACATCCAG | |
| GCCATCCAGCTCCCTGCCCTGCAGTACCAGCACGAGAGGCGCGCCAGCGACTGGGCTGTGGACAGCAACCTCTGT | |
| GGGGCCATGGACCCCTCCGACGGCATTGGAGCCCTCATCGAGGAGGAGCGCACCGCTGTCAAGCTCAACACTGGG | |
| CTCGCCCTGCACTGCCAGCAATTCTGGGCCATGTTCCTGAAGAAGGCCGCATACAGCTGGCGCGAGTGGAAAATG | |
| GTGGCGGCACAGGTCCTGGTGCCTCTGACCTGCGTCACCCTGGCCCTCCTGGCCATCAACTACTCCTCGGAGCTC | |
| TTCGACGACCCCATGCTGAGGCTGACCTTGGGCGAGTACGGCAGAACCGTCGTGCCCTTCTCAGTTCCCGGGACC | |
| TCCCAGCTGGGTCAGCAGCTGTCAGAGCATCTGAAAGACGCACTGCAGGCTGAGGGACAGGAGCCCCGCGAGGTG | |
| CTCGGTGACCTGGAGGAGTTCTTGATCTTCAGGGCTTCTGTGGAGGGGGGCGGCTTTAATGAGCGGTGCCTTGTG | |
| GCAGCGTCCTTCAGAGATGTGGGAGAGCGCACGGTCGTCAACGCCTTGTTCAACAACCAGGCGTACCACTCTCCA | |
| GCCACTGCCCTGGCCGTCGTGGACAACCTTCTGTTCAAGCTGCTGTGCGGGCCTCACGCCTCCATTGTGGTCTCC | |
| AACTTCCCCCAGCCCCGGAGCGCCCTGCAGGCTGCCAAGGACCAGTTTAACGAGGGCCGGAAGGGATTCGACATT | |
| GCCCTCAACCTGCTCTTCGCCATGGCATTCTTGGCCAGCACGTTCTCCATCCTGGCGGTCAGCGAGAGGGCCGTG | |
| CAGGCCAAGCATGTGCAGTTTGTGAGTGGAGTCCACGTGGCCAGTTTCTGGCTCTCTGCTCTGCTGTGGGACCTC | |
| ATCTCCTTCCTCATCCCCAGTCTGCTGCTGCTGGTGGTGTTTAAGGCCTTCGACGTGCGTGCCTTCACGCGGGAC | |
| GGCCACATGGCTGACACCCTGCTGCTGCTCCTGCTCTACGGCTGGGCCATCATCCCCCTCATGTACCTGATGAAC | |
| TTCTTCTTCTTGGGGGCGGCCACTGCCTACACGAGGCTGACCATCTTCAACATCCTGTCAGGCATCGCCACCTTC | |
| CTGATGGTCACCATCATGCGCATCCCAGCTGTAAAACTGGAAGAACTTTCCAAAACCCTGGATCACGTGTTCCTG | |
| GTGCTGCCCAACCACTGTCTGGGGATGGCAGTCAGCAGTTTCTACGAGAACTACGAGACGCGGAGGTACTGCACC | |
| TCCTCCGAGGTCGCCGCCCACTACTGCAAGAAATATAACATCCAGTACCAGGAGAACTTCTATGCCTGGAGCGCC | |
| CCGGGGGTCGGCCGGTTTGTGGCCTCCATGGCCGCCTCAGGGTGCGCCTACCTCATCCTGCTCTTCCTCATCGAG | |
| ACCAACCTGCTTCAGAGACTCAGGGGCATCCTCTGCGCCCTCCGGAGGAGGCGGACACTGACAGAATTATACACC | |
| CGGATGCCTGTGCTTCCTGAGGACCAAGATGTAGCGGACGAGAGGACCCGCATCCTGGCCCCCAGTCCGGACTCC | |
| CTGCTCCACACACCTCTGATTATCAAGGAGCTCTCCAAGGTGTACGAGCAGCGGGTGCCCCTCCTGGCCGTGGAC | |
| AGGCTCTCCCTCGCGGTGCAGAAAGGGGAGTGCTTCGGCCTGCTGGGCTTCAATGGAGCCGGGAAGACCACGACT | |
| TTCAAAATGCTGACCGGGGAGGAGAGCCTCACTTCTGGGGATGCCTTTGTCGGGGGTCACAGAATCAGCTCTGAT | |
| GTCGGAAAGGTGCGGCAGCGGATCGGCTACTGCCCGCAGTTTGATGCCTTGCTGGACCACATGACAGGCCGGGAG | |
| ATGCTGGTCATGTACGCTCGGCTCCGGGGCATCCCTGAGCGCCACATCGGGGCCTGCGTGGAGAACACTCTGCGG | |
| GGCCTGCTGCTGGAGCCACATGCCAACAAGCTGGTCAGGACGTACAGTGGTGGTAACAAGCGGAAGCTGAGCACC | |
| GGCATCGCCCTGATCGGAGAGCCTGCTGTCATCTTCCTGGACGAGCCGTCCACTGGCATGGACCCCGTGGCCCGG | |
| CGCCTGCTTTGGGACACCGTGGCACGAGCCCGAGAGTCTGGCAAGGCCATCATCATCACCTCCCACAGCATGGAG | |
| GAGTGTGAGGCCCTGTGCACCCGGCTGGCCATCATGGTGCAGGGGCAGTTCAAGTGCCTGGGCAGCCCCCAGCAC | |
| CTCAAGAGCAAGTTCGGCAGCGGCTACTCCCTGCGGGCCAAGGTGCAGAGTGAAGGGCAACAGGAGGCGCTGGAG | |
| GAGTTCAAGGCCTTCGTGGACCTGACCTTTCCAGGCAGCGTCCTGGAAGATGAGCACCAAGGCATGGTCCATTAC | |
| CACCTGCCGGGCCGTGACCTCAGCTGGGCGAAGGTTTTCGGTATTCTGGAGAAAGCCAAGGAAAAGTACGGCGTG | |
| GACGACTACTCCGTGAGCCAGATCTCGCTGGAACAGGTCTTCCTGAGCTTCGCCCACCTGCAGCCGCCCACCGCA | |
| GAGGAGGGGCGATGA | |
| Exemplified Human ABCA3 polypeptide | |
| SEQ ID NO: 36 | |
| MAVLRQLALLLWKNYTLQKRKVLVTVLELFLPLLFSGILIWLRLKIQSENVPNATIYPGQSIQELPLFFTFPPPG | |
| DTWELAYIPSHSDAAKTVTETVRRALVINMRVRGFPSEKDFEDYIRYDNCSSSVLAAVVFEHPENHSKEPLPLAV | |
| KYHLRFSYTRRNYMWTQTGSFFLKETEGWHTTSLFPLFPNPGPREPTSPDGGEPGYIREGFLAVQHAVDRAIMEY | |
| HADAATRQLFQRLTVTIKRFPYPPFIADPFLVAIQYQLPLLLLLSFTYTALTIARAVVQEKERRLKEYMRMMGLS | |
| SWLHWSAWFLLFFLFLLIAASFMTLLFCVKVKPNVAVLSRSDPSLVLAFLLCFAISTISFSFMVSTFFSKANMAA | |
| AFGGFLYFFTYIPYFFVAPRYNWMTLSQKLCSCLLSNVAMAMGAQLIGKFEAKGMGIQWRDLLSPVNVDDDFCFG | |
| QVLGMLLLDSVLYGLVTWYMEAVFPGQFGVPQPWYFFIMPSYWCGKPRAVAGKEEEDSDPEKALRNEYFEAEPED | |
| LVAGIKIKHLSKVFRVGNKDRAAVRDLNLNLYEGQITVLLGHNGAGKTTTLSMLTGLFPPTSGRAYISGYEISQD | |
| MVQIRKSLGLCPQHDILFDNLTVAEHLYFYAQLKGLSRQKCPEEVKQMLHIIGLEDKWNSRSRFLSGGMRRKLSI | |
| GIALIAGSKVLILDEPTSGMDAISRRAIWDLLQRQKSDRTIVLTTHEMDEADLLGDRIAIMAKGELQCCGSSLFL | |
| KQKYGAGYHMTLVKEPHCNPEDISQLVHHHVPNATLESSAGAELSFILPRESTHRFEGLFAKLEKKQKELGIASF | |
| GASITTMEEVFLRVGKLVDSSMDIQAIQLPALQYQHERRASDWAVDSNLCGAMDPSDGIGALIEEERTAVKLNTG | |
| LALHCQQFWAMELKKAAYSWREWKMVAAQVLVPLTCVTLALLAINYSSELFDDPMLRLTLGEYGRTVVPFSVPGT | |
| SQLGQQLSEHLKDALQAEGQEPREVLGDLEEFLIFRASVEGGGENERCLVAASFRDVGERTVVNALENNQAYHSP | |
| ATALAVVDNLLFKLLCGPHASIVVSNFPQPRSALQAAKDQFNEGRKGFDIALNLLFAMAFLASTFSILAVSERAV | |
| QAKHVQFVSGVHVASFWLSALLWDLISFLIPSLLLLVVFKAFDVRAFTRDGHMADTLLLLLLYGWAIIPLMYLMN | |
| FFFLGAATAYTRLTIFNILSGIATFLMVTIMRIPAVKLEELSKTLDHVFLVLPNHCLGMAVSSFYENYETRRYCT | |
| SSEVAAHYCKKYNIQYQENFYAWSAPGVGRFVASMAASGCAYLILLFLIETNLLQRLRGILCALRRRRTLTELYT | |
| RMPVLPEDQDVADERTRILAPSPDSLLHTPLIIKELSKVYEQRVPLLAVDRLSLAVQKGECFGLLGENGAGKTTT | |
| FKMLTGEESLTSGDAFVGGHRISSDVGKVRQRIGYCPQFDALLDHMTGREMLVMYARLRGIPERHIGACVENTLR | |
| GLLLEPHANKLVRTYSGGNKRKLSTGIALIGEPAVIFLDEPSTGMDPVARRLLWDTVARARESGKAIIITSHSME | |
| ECEALCTRLAIMVQGQFKCLGSPQHLKSKFGSGYSLRAKVQSEGQQEALEEFKAFVDLTFPGSVLEDEHQGMVHY | |
| HLPGRDLSWAKVFGILEKAKEKYGVDDYSVSQISLEQVFLSFAHLQPPTAEEGR | |
| Exemplified WPRE component (mWPRE) | |
| SEQ ID NO: 37 | |
| 1 | GGGCCCAATC AACCTCTGGA TTACAAAATT TGTGAAAGAT TGACTGGTAT TCTTAACTAT |
| 61 | GTTGCTCCTT TTACGCTATG TGGATACGCT GCTTTAATGC CTTTGTATCA TGCTATTGCT |
| 121 | TCCCGTATGG CTTTCATTTT CTCCTCCTTG TATAAATCCT GGTTGCTGTC TCTTTATGAG |
| 181 | GAGTTGTGGC CCGTTGTCAG GCAACGTGGC GTGGTGTGCA CTGTGTTTGC TGACGCAACC |
| 241 | CCCACTGGTT GGGGCATTGC CACCACCTGT CAGCTCCTTT CCGGGACTTT CGCTTTCCCC |
| 301 | CTCCCTATTG CCACGGCGGA ACTCATCGCC GCCTGCCTTG CCCGCTGCTG GACAGGGGCT |
| 361 | CGGCTGTTGG GCACTGACAA TTCCGTGGTG TTGTCGGGGA AATCATCGTC CTTTCCTTGG |
| 421 | CTGCTCGCCT GTGTTGCCAC CTGGATTCTG CGCGGGACGT CCTTCTGCTA CGTCCCTTCG |
| 481 | GCCCTCAATC CAGCGGACCT TCCTTCCCGC GGCCTGCTGC CGGCTCTGCG GCCTCTTCCG |
| 541 | CGTCTTCGCC TTCGCCCTCA GACGAGTCGG ATCTCCCTTT GGGCCGCCTC CCCGCAAGCT |

### EXAMPLES

The invention is now described with reference to the Examples below. These are not limiting on the scope of the invention, and a person skilled in the art would be appreciate that suitable equivalents could be used within the scope of the present invention. Thus, the Examples may be considered component parts of the invention, and the individual aspects described therein may be considered as disclosed independently, or in any combination.

### Example 1—Identification of Exogenous Signal Peptides for Increased Expression in Airway Cells

A bioinformatic search was performed for proteins highly secreted from the lungs to identify signal peptides that drive strong secretion from airway and lung cells. Proteins produced predominantly in the lungs and secreted to high concentrations into either blood or epithelial lining fluid were selected, and the signal peptides were identified.

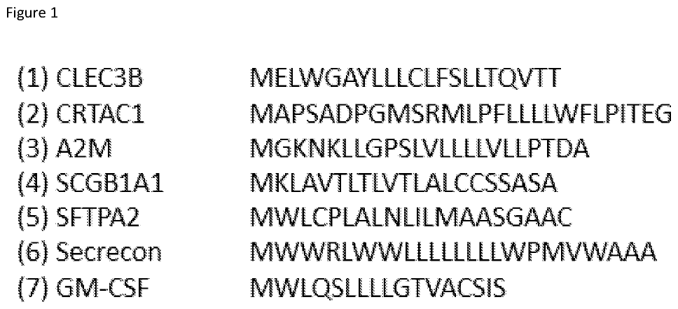

As shown in FIG. 1, this bioinformatic analysis of proteins secreted from the lungs into the circulation or epithelial lining fluid identified five strong endogenous lung signal peptide candidates: (1) Fibronectin (CLEC3B), (2) Cartilage Acidic Protein 1 (CRTAC1), (3) Alpha-2-macroglobulin (A2M), (4) Uteroglobin (SCGB1A1), and (5) Pulmonary surfactant associated protein A (SFTPA2), together with the strong synthetic signal peptide, (6) Secrecon, and the endogenous signal peptide of a therapeutically relevant protein, (7) Granulocyte-macrophage Colony-stimulating factor (GM-CSF).

To isolate the precise signal peptide candidates, SignalP-5.0 was used to analyse the first 50 amino acids from the amino acid sequences of the protein candidates to identify cleavage sites. This is exemplified in FIG. 2 for A2M. In particular, a strong cleavage site was identified at alanine 23. The first 23 amino acids of the A2M protein were reverse translated and codon optimised for translation in human cells. The process was repeated to identify the amino acid sequences of the CRTAC1, AAT, CLEC3B, SCGB1A1, SFTPA2, GM-CSF and IDS signal peptides (data not shown).

As signal peptide sequences identified by this search lead to high level secretion from airway and lung cells, they are expected to have the capacity to drive high secretion of exogenous proteins from airway and lung cells as well. Similarly, as the search was conducted from human databases, and all signal peptide sequences come from human proteins, it is expected that the signal peptides will drive high secretion in human airways.

### Example 2—Production of Viral Vectors Comprising Exogenous Signal Peptides and Transgenes

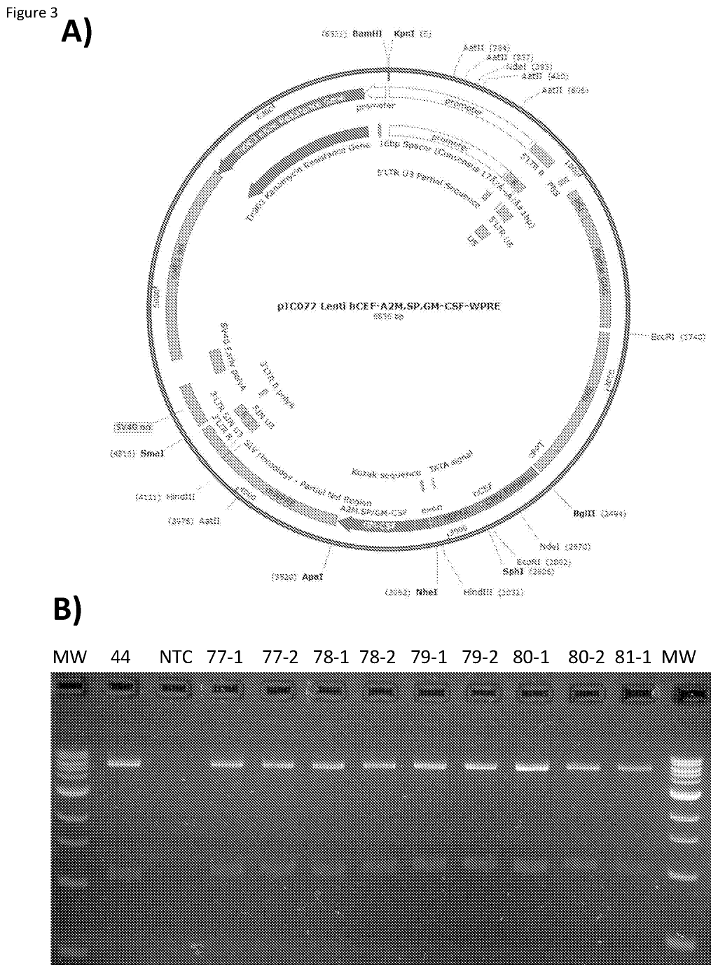

The signal peptides identified in Example 1 were codon optimized and fused to therapeutic proteins such as GM-CSF or alpha-1-antitrypsin (AAT). As exemplified for A2M signal peptide and GM-CSF in FIG. 3, the signal peptides were cloned into a lentiviral transfer plasmid containing a therapeutic protein and an expression cassette. The clones were screened by a HindIII restriction digest, producing two bands as expected for all plasmids generated (FIG. 3B).

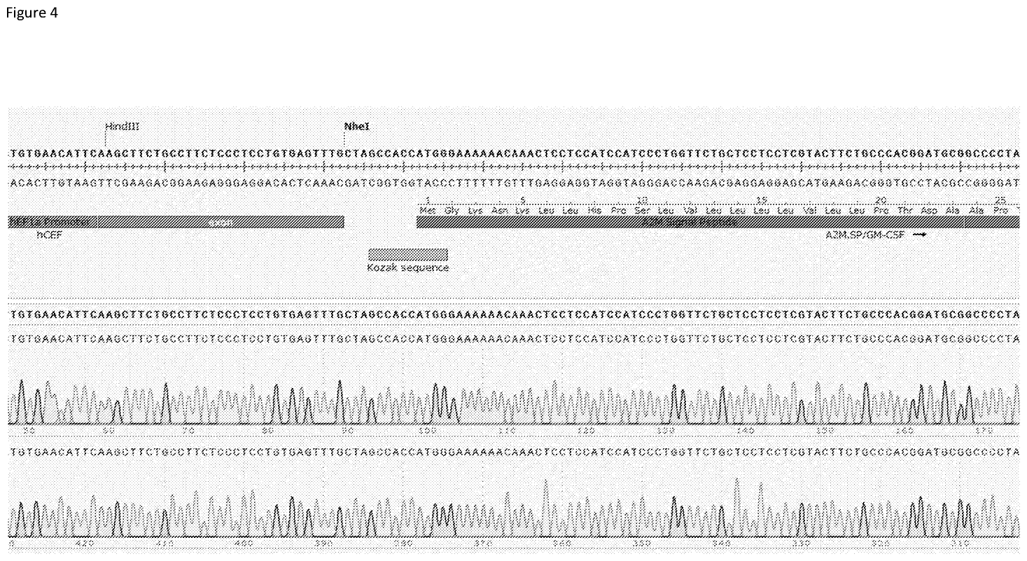

Proper fusion of the signal peptides to the therapeutic transgene was confirmed by sanger sequencing providing 2-fold coverage of the signal peptide and fusion site with the transgene. This is exemplified in FIG. 4 for GM-CSF.

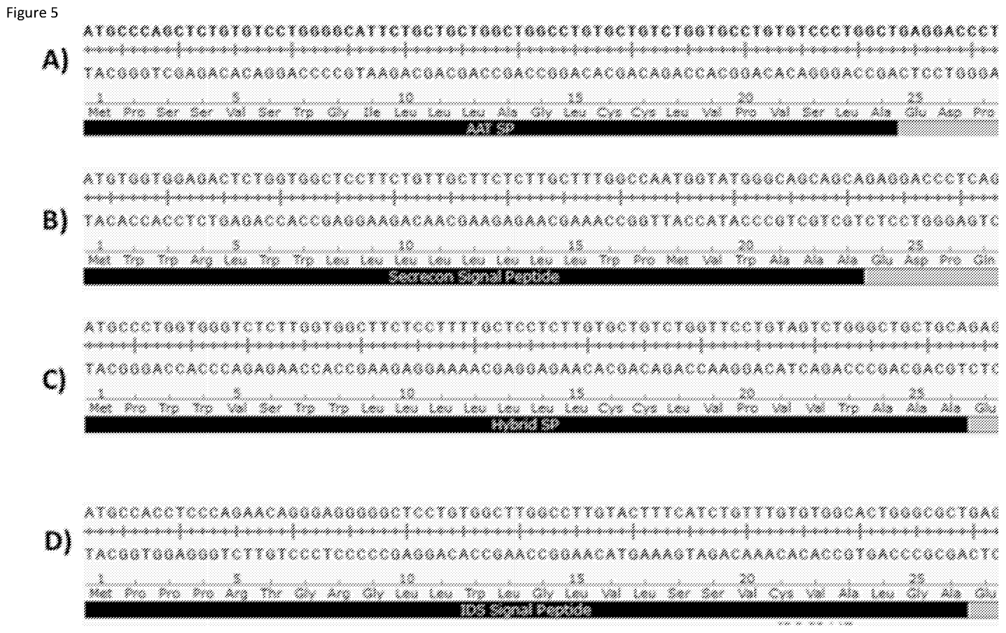

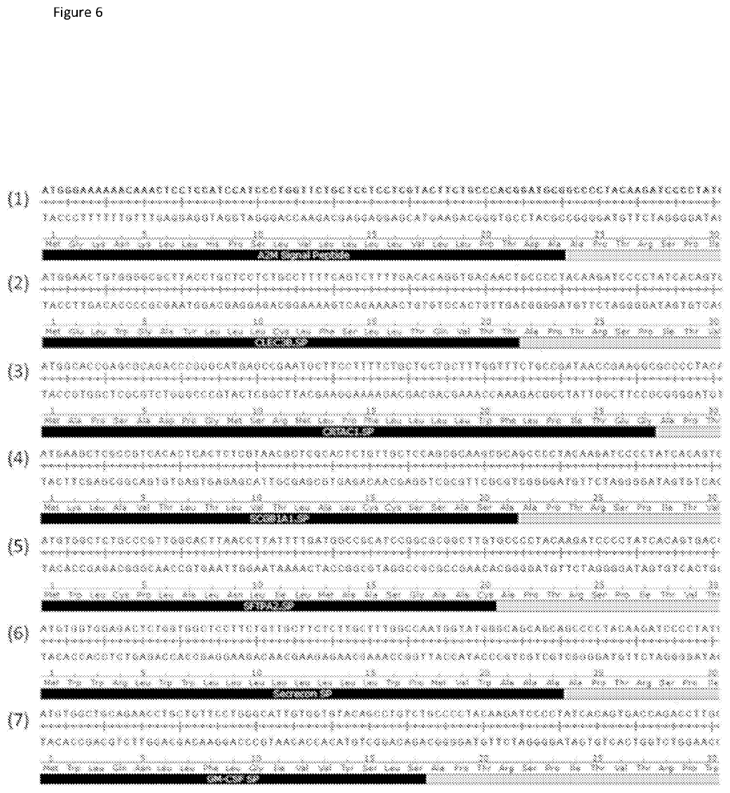

FIG. 5 illustrates cassettes generated in which exogenous signal peptides from Example 1 were fuse to AAT. FIG. 6 illustrates further cassettes generated in which exogenous signal peptides identified in Example 1 were fused to GM-SF.

### Example 3—Use of Exogenous Signal Peptides Increased AAT Secretion from Transfected HEK283T Cells

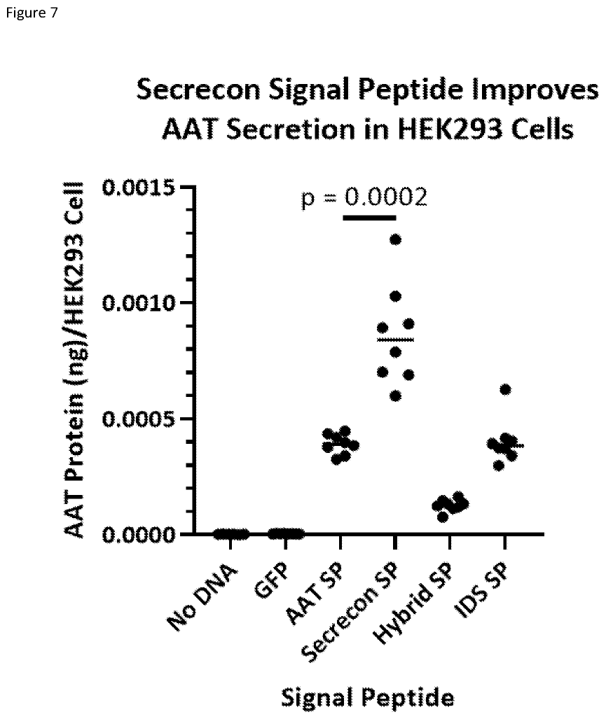

Four signal peptides were selected for initial in vitro testing: the endogenous signal peptide sequence from alpha-1-antitrypsin, a synthetic sequence called Secrecon, a hybrid of the alpha-1-antitrypsin and Secrecon signal peptides, and the signal peptide from Iduronate 2-Sulfatase (FIG. 7).

HEK293T cells were transiently transfected with plasmids containing candidate signal peptides fused to the coding sequence of SERPINA1 (which encodes for AAT). Two days after the transfection, media was collected from transduced cells and AAT content was quantified by ELISA. Data was analysed with a Kruskal-Wallis test comparing all groups except NTC (non-transduced control). As can be seen in FIG. 7, in this experiment, the Secrecon signal peptide significantly increased AAT expression compared with the native AAT signal peptide.

### Example 4—Lung Signal Peptides Modify GM-CSF Secretion from Transfected HEK293T Cells

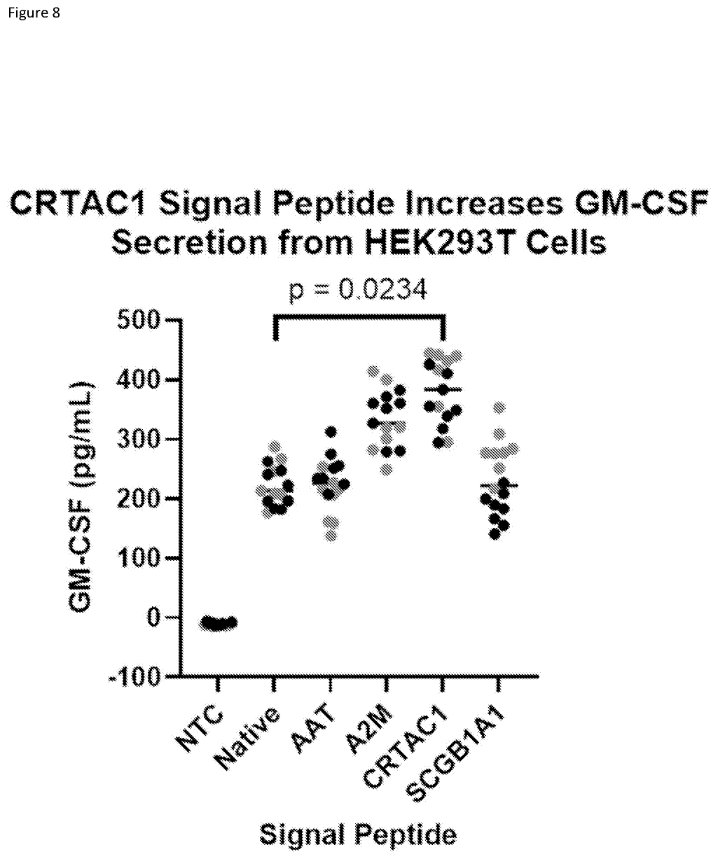

To screen lung signal peptides HEK293T cells were transiently transfected with plasmids containing candidate signal peptides fused to the coding sequence of GM-CSF. Two days after the transfection, media was collected from transduced cells and GM-CSF content was quantified by ELISA. The transfection experiment was performed twice (n=7-8 wells per experiment). Bars represent median values. Data was analysed with a Kruskal-Wallis test comparing all groups except NTC (non-transduced control). As can be seen in FIG. 8, in this experiment, the AAT, A2M, and SCGB1A1 signal peptides all performed comparably or better than the native GM-CSF signal peptide. The CRTAC1 signal peptide increased GM-CSF expression by a median of 1.76-fold compared to the native

### Example 5—Lung Signal Peptides Modify GM-CSF Secretion from Transfected Human Air Liquid Interface Cultures Using Non-Viral Vectors

Human air liquid interface (ALI) cultures, a model of human airways, are transiently transfected with plasmids encoding the signal peptide-GM-CSF fusions (n=6 per signal peptide). One week after transfection the apical surface of the ALIs is incubated with culture media for one hour at 37° C., collected, and assayed by ELISA for GM-CSF protein. Use of exogenous signal peptides, particularly CRTAC1, increases secretion of GM-CSF compared with untransfected ALIs.

### Example 6—Lung Signal Peptides Modify GM-CSF Secretion from Transfected Mice Using Non-Viral Vectors

C57BL/6J mice are transiently transfected with plasmids encoding the signal peptide-GM-CSF fusions (n=6 per signal peptide). One week after nasal administration bronchoalveolar lavage fluid is collected and GM-CSF levels measured by ELISA. Use of exogenous signal peptides, particularly CRTAC1, increases BALF levels of GM-CSF compared with untransfected ALIs, indicating that secretion of GM-CSF is increased by the exogenous signal peptides.

### Example 7—Exogenous Signal Peptides Modify AAT Secretion from Transfected Human Air Liquid Interface Cultures Using Viral Vectors

Human air liquid interface (ALI) cultures, a model of human airways, are transfected with rSIV.F/HN lentiviral vectors encoding the signal peptide-AAT fusions (n=6 per signal peptide). One week after transfection the apical surface of the ALIs is incubated with culture media for one hour at 37° C., collected, and assayed by ELISA for AAT protein. Use of exogenous signal peptides, particularly secrecon, increases secretion of AAT compared with untransfected ALIs.

### Example 8—Exogenous Signal Peptides Modify AAT Secretion from Transfected Mice Using Viral Vectors

C57BL/6J mice are transfected with rSIV.F/HN lentiviral vectors encoding the signal peptide-AAT fusions (n=6 per signal peptide). One week after nasal administration bronchoalveolar lavage fluid is collected and AAT levels measured by ELISA. Use of exogenous signal peptides, particularly secrecon, increases BALF levels of AAT compared with untransfected ALIs, indicating that secretion of GM-CSF is increased by the exogenous signal peptides.

### Example 9—Lung Signal Peptides Modify GM-CSF Secretion from Transfected Human Air Liquid Interface Cultures Using Viral Vectors

Human ALI cultures, were transduced with VSV-G pseudotyped lentiviral vectors encoding the signal peptide-GM-CSF fusions (n=6 per signal peptide). One week after transduction the apical surface of the ALIs was incubated with culture media for one hour at 37° C., collected, and assayed by ELISA for GM-CSF protein. Use of exogenous signal peptides CRTAC1 and SCGB1A1, significantly increased secretion of GM-CSF compared with untransduced ALIs (NTCs). Use of the CRTAC1 signal peptide increased GM-CSF secretion by 2.3 times compared with the native signal peptide. Use of the SCGB1A1 signal peptide increased GM-CSF secretion by 2.4 times compared with the native signal peptide.

### Example 10—SCGB1A1 Signal Peptide Increases AAT Secretion from Transfected Human Air Liquid Interface Cultures Using Viral Vectors

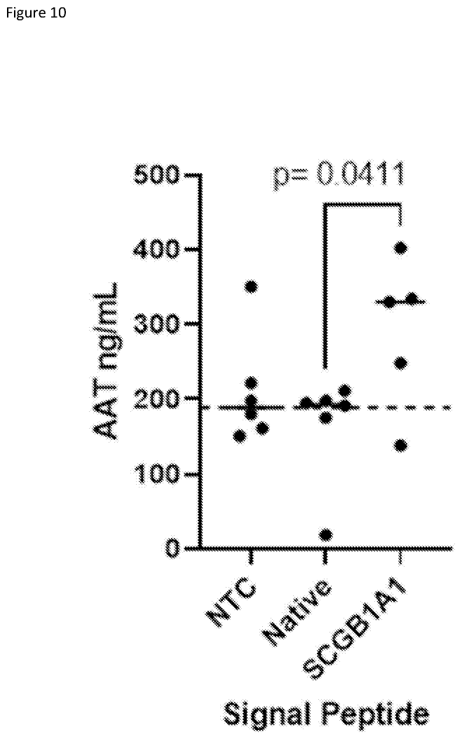

Human ALI cultures were transfected with VSV-G pseudotyped lentiviral vectors encoding the signal peptide-AAT fusions (n=6 per signal peptide). One week after transfection the apical surface of the ALIs is incubated with culture media for one hour at 37° C., collected, and assayed by ELISA for AAT protein. Use of the exogenous SCGB1A1 signal peptide significantly increased secretion of AAT compared with untransduced ALIs (NTC) or ALIs transduced with AAT fused with its native signal peptide (FIG. 10).

1. A nucleic acid cassette comprising:
(a) a nucleic acid sequence encoding an exogenous signal peptide; and
(b) a nucleic acid sequence encoding a therapeutic protein;
wherein the exogenous signal peptide (i) increases secretion of the therapeutic protein from airway cells and/or (ii) increases insertion of the therapeutic protein into the cell membrane of airway cells.

2. The nucleic acid cassette of claim 1, wherein the exogenous signal peptide is capable of:
(a) increasing secretion of the therapeutic protein as compared to secretion of the therapeutic protein without the exogenous signal peptide; and/or
(b) increasing secretion of the therapeutic protein as compared to secretion of the therapeutic protein with its endogenous signal peptide.

3. The nucleic acid cassette of claim 1, wherein the exogenous signal peptide is capable of:
(a) increasing insertion of the therapeutic protein into the cell membrane of airway cells as compared to membrane insertion of the therapeutic protein without the exogenous signal peptide; and/or
(b) increasing insertion of the therapeutic protein into the cell membrane of airway cells as compared to membrane insertion of the therapeutic protein with its endogenous signal peptide.

4. The nucleic acid cassette of any one of claims 1 to 3, wherein the exogenous signal peptide is a signal peptide that drives high secretion from and/or membrane insertion by airway cells, wherein optionally the exogenous signal peptide is selected from:
(a) a cartilage acidic protein 1 (CRTAC1) signal peptide;
(b) a uteroglobin (SCGB1A1) signal peptide;
(c) an alpha-2-macroglobulin (A2M) signal peptide;
(d) a synthetic signal peptide;
(e) a pulmonary surfactant associated protein A (SFTPA2) signal peptide;
(f) a fibronectin (CLEC3B) signal peptide;
(g) an alpha-1-antitrypsin (AAT) signal peptide;
(h) a granulocyte-macrophage Colony-stimulating factor (GM-CSF) signal peptide;
(i) an iduronate 2-sulfatase (IDS) signal peptide; and
(j) a hybrid signal peptide, optionally a hybrid of an ATT signal peptide and a synthetic signal peptide.

5. The nucleic acid cassette of any one of the preceding claims, wherein the exogenous signal peptide comprises or consists of:
(a) an amino acid sequence having at least 90% identity to an amino acid selected from the group consisting of: SEQ ID NOs: 1-12; or
(b) an amino acid sequence selected from the group consisting of: SEQ ID NOs: 1-12.

6. The nucleic acid cassette of any one of the preceding claims, wherein the nucleic acid sequence encoding the exogenous signal peptide is 5′ of the nucleic acid sequence encoding the therapeutic protein.

7. The nucleic acid cassette of any one of the preceding claims, which further comprises a promoter configured to express the nucleic acid sequence encoding the exogenous signal peptide and the therapeutic protein.

8. The nucleic acid cassette of claim 7, wherein the promote is selected from the group consisting of a hybrid human cytomegalovirus (CMV) enhancer/elongation factor 1 a (EF1 a) promoter (hCEF), a CMV promoter and an EF1 a promoter, preferably a hCEF promoter.

9. The nucleic acid cassette of any one of the preceding claims further comprising:
(a) a translation initiation sequence; and/or
(b) an internal ribosome entry sequence (IRES).

10. The nucleic acid cassette of any one of the preceding claims, wherein the therapeutic protein is:
(a) a secreted therapeutic protein selected from: AAT, Factor VIII, Surfactant Protein B (SP-B), Factor VII, Factor IX, Factor X, Factor XI, van Willebrand Factor, Granulocyte-Macrophage Colony-Stimulating Factor (GM-CSF), Surfactant Protein C (SP-C), decorin, an anti-inflammatory protein (e.g. IL-10 or TGFβ) or monoclonal antibody, an anti-inflammatory decoy, or a monoclonal antibody against an infectious agent; or
(b) CFTR, TRIM72, CSF2RA, CSF2RB or ATP-binding cassette sub-family A member 3 (ABCA3).

11. The nucleic acid cassette of any one of the preceding claims, wherein the airway cells are:
(a) lung cells; and/or
(b) selected from epithelial cells, basal cells, submucosal gland duct cells, club cells, neuroendocrine cells, bronchoalveolar stem cells, submucosal acinar cells, ionocytes, type I pneumocytes and/or type II pneumocytes.

12. A gene therapy vector, comprising a nucleic acid cassette as defined in any one of the preceding claims.

13. The gene therapy vector of claim 12, which is a non-viral vector, wherein optionally:
(a) the non-viral vector is a plasmid; and/or
(b) the non-viral vector is comprised in a cationic liposome, which preferably comprises GL67A.

14. The gene therapy vector of claim 12, which is a viral vector, optionally selected from:
(a) a lentiviral vector;
(b) an AAV vector;
(c) an adenoviral vector; and
(d) a sendai virus vector.

15. The gene therapy vector of claim 14, which is a lentiviral vector that is pseudotyped with hemagglutinin-neuraminidase (HN) and fusion (F) proteins from a respiratory paramyxovirus.

16. The gene therapy vector of claim 15, wherein the respiratory paramyxovirus is a Sendai virus.

17. The gene therapy vector of any one of claims 14-16, wherein the lentiviral vector is selected from the group consisting of a Human immunodeficiency virus (HIV) vector, a Simian immunodeficiency virus (SIV) vector, a Feline immunodeficiency virus (FIV) vector, an Equine infectious anaemia virus (EIAV) vector, and a Visna/maedi virus vector.

18. The gene therapy vector of claim 17, wherein the lentiviral vector is a SIV vector.

19. A method of expressing a secreted therapeutic protein in a target cell, comprising delivering a nucleic acid cassette as defined in any one of claims 1-11 or a gene therapy vector as defined in any one of claims 12-18 into the target cells.

20. The method of claim 19, wherein said delivering comprises integrating said nucleic acid cassette or gene therapy vector into said target cell's genome.

21. A gene therapy vector as defined in any one of claims 12-18 for use in a method of treating a disease.

22. The gene therapy vector for use of claim 21, wherein the disease is a genetic disease.

23. The gene therapy vector for use of claim 21 or 22, wherein the disease is:
(a) a respiratory disease, particularly a genetic respiratory disease; or
(b) a cardiovascular disease or blood disorder, particularly a genetic cardiovascular disease or blood disorder.

24. The gene therapy vector for use of any one of claims 21-23, wherein the disease is selected from cystic fibrosis (CF); Primary Ciliary Dyskinesia (PCD); Surfactant Protein B (SP-B) Deficiency; Alpha 1-antitrypsin Deficiency (A1AD); Pulmonary Alveolar Proteinosis (PAP); Chronic obstructive pulmonary disease (COPD); Pulmonary surfactant metabolism dysfunction 2 (SMDP2); Pulmonary surfactant metabolism dysfunction 3 (SMDP3); Acute respiratory distress syndrome (ARDS); COVID-19; a pulmonary fibrotic disease; a pulmonary allergic condition; a pulmonary bacterial infection; lung cancer; a dysplastic change in the lungs; and haemophilia.

25. A cell comprising a nucleic acid cassette as defined in any one of claims 1-11 or a gene therapy vector as defined in any one of claims 12-18.

26. A composition comprising a nucleic acid cassette as defined in any one of claims 1-11 or a gene therapy vector as defined in any one of claims 12-18 and a pharmaceutically acceptable carrier, diluent or excipient.


SEQUENCE LISTING (37 sequences)

SEQ ID NO: 1 - protein, 27 aa, Homo sapiens
MAPSADPGMSRMLPFLLLLWFLPITEG

SEQ ID NO: 2 - protein, 24 aa, Homo sapiens
MPSSVSWGILLLAGLCCLVPVSLA

SEQ ID NO: 3 - protein, 21 aa, Homo sapiens
MELWGAYLLLCLFSLLTQVTT

SEQ ID NO: 4 - protein, 23 aa, Homo sapiens
MGKNKLLGPSLVLLLLVLLPTDA

SEQ ID NO: 5 - protein, 21 aa, Homo sapiens
MKLAVTLTLVTLALCCSSASA

SEQ ID NO: 6 - protein, 20 aa, Homo sapiens
MWLCPLALNLILMAASGAAC

SEQ ID NO: 7 - protein, 17 aa, Homo sapiens
MWLQSLLLLGTVACSIS

SEQ ID NO: 8 - protein, 17 aa, Mus musculus
MWLQNLLFLGIVVYSLS

SEQ ID NO: 9 - protein, 26 aa, Homo sapiens
MPPPRTGRGLLWLGLVLSSVCVALGA

SEQ ID NO: 10 - protein, 21 aa, Artificial Sequence
  Secrecon signal peptide
MWWRLWWLLLLLLLLWPMVWA

SEQ ID NO: 11 - protein, 23 aa, Artificial Sequence
  Secrecon signal peptide
MWWRLWWLLLLLLLLWPMVWAAA

SEQ ID NO: 12 - protein, 26 aa, Artificial Sequence
  Secrecon-AAT hybrid signal peptide
MPWWVSWWLLLLLLLCCLVPVVWAAA

SEQ ID NO: 13 - DNA, 574 bp, Artificial Sequence
  hCEF promoter
agatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagc

SEQ ID NO: 14 - DNA, 873 bp, Cauliflower mosaic virus
ccgcggagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggctagc

SEQ ID NO: 15 - DNA, 395 bp, Homo sapiens
agatccatatccgcggcaattttaaaagaaagggaggaatagggggacagacttcagcagagagactaattaatataataacaacacaattagaaatacaacatttacaaaccaaaattcaaaaaattttaaattttagagccgcggagatcccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagc

SEQ ID NO: 16 - DNA, 4443 bp, Artificial Sequence
  soCFTR2
atgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtga

SEQ ID NO: 17 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 18 - DNA, 435 bp, Homo sapiens
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 19 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 20 - DNA, 426 bp, Mus musculus
atgtggctgcagaacctgctgttcctgggcattgtggtgtacagcctgtctgcccctacaagatcccctatcacagtgaccagaccttggaaacatgtggaagccatcaaagaggccctgaatctgctggatgacatgcctgtgacactgaatgaagaggtggaagtggtgtccaatgagttcagcttcaagaaactgacctgtgtgcagaccaggctgaagatttttgagcagggcctgagaggcaacttcaccaagctgaaaggggctctgaacatgacagccagctactaccagacctactgtcctcctacacctgagacagactgtgaaacccaagtgaccacctatgctgacttcattgacagcctcaagaccttcctgacagacatcccctttgagtgcaagaaacctggccagaagtga

SEQ ID NO: 21 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 22 - DNA, 1257 bp, Homo sapiens
atgcccagctctgtgtcctggggcattctgctgctggctggcctgtgctgtctggtgcctgtgtccctggctgaggaccctcagggggatgctgcccagaaaacagacacctcccaccatgaccaggaccaccccaccttcaacaagatcacccccaacctggcagagtttgccttcagcctgtacagacagctggcccaccagagcaacagcaccaacatctttttcagccctgtgtccattgccacagcctttgccatgctgagcctgggcaccaaggctgacacccatgatgagatcctggaaggcctgaacttcaacctgacagagatccctgaggcccagatccatgagggcttccaggaactgctgagaaccctgaaccagccagacagccagctgcagctgacaacaggcaatgggctgttcctgtctgagggcctgaagctggtggacaagtttctggaagatgtgaagaagctgtaccactctgaggccttcacagtgaactttggggacacagaagaggccaagaaacagatcaatgactatgtggaaaagggcacccagggcaagattgtggaccttgtgaaagagctggacagggacactgtgtttgcccttgtgaactacatcttcttcaagggcaagtgggagaggccctttgaagtgaaggacactgaggaagaggacttccatgtggaccaagtgaccacagtgaaggtgccaatgatgaagagactggggatgttcaatatccagcactgcaagaaactgagcagctgggtgctgctgatgaagtacctgggcaatgctacagccatattctttctgcctgatgagggcaagctgcagcacctggaaaatgagctgacccatgacatcatcaccaaatttctggaaaatgaggacagaagatctgccagcctgcatctgcccaagctgagcatcacaggcacatatgacctgaagtctgtgctgggacagctgggaatcaccaaggtgttcagcaatggggcagacctgagtggagtgacagaggaagcccctctgaagctgtccaaggctgtgcacaaggcagtgctgaccattgatgagaagggcacagaggctgctggggccatgtttctggaagccatccccatgtccatccccccagaagtgaagttcaacaagccctttgtgttcctgatgattgagcagaacaccaagagccccctgttcatgggcaaggttgtgaaccccacccagaaatga

SEQ ID NO: 23 - DNA, 1257 bp, Homo sapiens
tacgggtcgagacacaggaccccgtaagacgacgaccgaccggacacgacagaccacggacacagggaccgactcctgggagtccccctacgacgggtcttttgtctgtggagggtggtactggtcctggtggggtggaagttgttctagtgggggttggaccgtctcaaacggaagtcggacatgtctgtcgaccgggtggtctcgttgtcgtggttgtagaaaaagtcgggacacaggtaacggtgtcggaaacggtacgactcggacccgtggttccgactgtgggtactactctaggaccttccggacttgaagttggactgtctctagggactccgggtctaggtactcccgaaggtccttgacgactcttgggacttggtcggtctgtcggtcgacgtcgactgttgtccgttacccgacaaggacagactcccggacttcgaccacctgttcaaagaccttctacacttcttcgacatggtgagactccggaagtgtcacttgaaacccctgtgtcttctccggttctttgtctagttactgatacaccttttcccgtgggtcccgttctaacacctggaacactttctcgacctgtccctgtgacacaaacgggaacacttgatgtagaagaagttcccgttcaccctctccgggaaacttcacttcctgtgactccttctcctgaaggtacacctggttcactggtgtcacttccacggttactacttctctgacccctacaagttataggtcgtgacgttctttgactcgtcgacccacgacgactacttcatggacccgttacgatgtcggtataagaaagacggactactcccgttcgacgtcgtggaccttttactcgactgggtactgtagtagtggtttaaagaccttttactcctgtcttctagacggtcggacgtagacgggttcgactcgtagtgtccgtgtatactggacttcagacacgaccctgtcgacccttagtggttccacaagtcgttaccccgtctggactcacctcactgtctccttcggggagacttcgacaggttccgacacgtgttccgtcacgactggtaactactcttcccgtgtctccgacgaccccggtacaaagaccttcggtaggggtacaggtaggggggtcttcacttcaagttgttcgggaaacacaaggactactaactcgtcttgtggttctcgggggacaagtacccgttccaacacttggggtgggtctttact

SEQ ID NO: 24 - protein, 442 aa, Homo sapiens
MPSSVSWGILLLAGLCCLVPVSLAEDPQGDAAQKTDTSHHDQDHPTFAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 25 - DNA, 5013 bp, Artificial Sequence
  FVIII N6 transgene
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaacagcaggcaccccagcaccaggcagaagcagttcaatgccaccaccatccctgagaatgacatagagaagacagacccatggtttgcccaccggacccccatgcccaagatccagaatgtgagcagctctgacctgctgatgctgctgaggcagagccccaccccccatggcctgagcctgtctgacctgcaggaggccaagtatgaaaccttctctgatgaccccagccctggggccattgacagcaacaacagcctgtctgagatgacccacttcaggccccagctgcaccactctggggacatggtgttcacccctgagtctggcctgcagctgaggctgaatgagaagctgggcaccactgctgccactgagctgaagaagctggacttcaaagtctccagcaccagcaacaacctgatcagcaccatcccctctgacaacctggctgctggcactgacaacaccagcagcctgggcccccccagcatgcctgtgcactatgacagccagctggacaccaccctgtttggcaagaagagcagccccctgactgagtctgggggccccctgagcctgtctgaggagaacaatgacagcaagctgctggagtctggcctgatgaacagccaggagagcagctggggcaagaatgtgagcagcagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 26 - DNA, 4425 bp, Artificial Sequence
  FVIII V3 transgene
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 27 - DNA, 5013 bp, Artificial Sequence
  Complementary strand to FVIII transgene (N6)
tacgtctaactcgactcgtggacgaagaaggacacggacgactccaagacgaagagacggtggtcctctatgatggacccccgacacctcgactcgaccctgatgtacgtcagactggaccccctcgacggacacctacggtccaagggggggtctcacgggttctcgaaggggaagttgtggagacaccacatgttcttctgggacaaacacctcaagtgactggtggacaagttgtaacggttcgggtccggggggacctacccggacgacccggggtggtaggtccgactccacatactgtgacaccactagtgggacttcttgtaccggtcggtgggacactcggacgtacgacacccccactcgatgaccttccggagactcccccgactcatactactggtctggtcggtctccctcttcctcctactgttccacaagggacccccgtcggtgtggatacacaccgtccacgacttcctcttaccggggtaccggagactgggggacacggactggatgtcgatggactcggtacacctggaccacttcctggacttgagaccggactaaccccgggacgaccacacgtccctcccgtcggaccggttcctcttctgggtctgggacgtgttcaagtaggacgacaaacgacacaaactactcccgttctcgaccgtgagactttggttcttgtcggactacgtcctgtccctacgacggagacggtcccggaccgggttctacgtgtgacacttaccgatacacttgtcctcggacggaccggactaaccgacggtgtccttcagacacatgaccgtacactaaccgtacccgtggtggggactccacgtgtcgtagaaggacctcccggtgtggaaggaccagtccttggtgtccgtccggtcggacctctagtcggggtagtggaaggactgacgggtctgggacgactacctggacccggtcaaggacgacaagacggtgtagtcgtcggtggtcgtactaccgtacctccggatacacttccacctgtcgacgggactcctcggggtcgactcctacttcttgttactcctccgactcctgatactactactggactgactgagactctacctacaccactccaaactactactgttgtcggggtcgaagtaggtctagtccagacaccggttcttcgtggggttctggacccacgtgatgtaacgacgactcctcctcctgaccctgatacggggggaccacgaccggggactactgtcctcgatgttctcggtcatggacttgttaccgggggtctcctaaccgtccttcatgttcttccagtccaagtaccggatgtgactactttggaagttctggtccctccggtaggtcgtactcagaccgtaggacccgggggacgacatacccctccaccccctgtgggacgactagtagaagttcttggtccggtcgtccgggatgttgtagatgggggtaccgtagtgactacactccggggacatgtcgtcctccgacgggttcccccacttcgtggacttcctgaaggggtaggacggacccctctagaagttcatgttcacctgacactgacacctcctaccggggtggttcagactggggtccacggactggtctatgatgtcgtcgaaacacttgtacctctccctggaccggagaccggactaaccgggggacgactagacgatgttcctcagacacctggtctccccgttggtctagtacagactgttctccttacactaggacaagagacacaaactactcttgtcctcgaccatggactgactcttgtaggtctccaaggacgggttgggacgaccccacgtcgacctcctgggactcaaggtccggtcgttgtagtacgtgtcgtagttaccgatacacaaactgtcggacgtcgacagacacacggacgtactccaccggatgaccatgtaggactcgtaaccccgggtctgactgaaggacagacacaagaagagaccgatgtggaagttcgtgttctaccacatactcctgtgggactgggacaaggggaagagacccctctgacacaagtactcgtacctcttgggaccggacacctaagacccgacggtgttgagactgaagtccttgtccccgtactgacgggacgactttcagaggtcgacactgttcttgtgacccctgatgatactcctgtcgatactcctgtagagacggatggacgactcgttcttgttacggtaactcgggtcctcgaagtcggtcttgtcgtccgtggggtcgtggtccgtcttcgtcaagttacggtggtggtagggactcttactgtatctcttctgtctgggtaccaaacgggtggcctgggggtacgggttctaggtcttacactcgtcgagactggacgactacgacgactccgtctcggggtggggggtaccggactcggacagactggacgtcctccggttcatactttggaagagactactggggtcgggaccccggtaactgtcgttgttgtcggacagactctactgggtgaagtccggggtcgacgtggtgagacccctgtaccacaagtggggactcagaccggacgtcgactccgacttactcttcgacccgtggtgacgacggtgactcgacttcttcgacctgaagtttcagaggtcgtggtcgttgttggactagtcgtggtaggggagactgttggaccgacgaccgtgactgttgtggtcgtcggacccgggggggtcgtacggacacgtgatactgtcggtcgacctgtggtgggacaaaccgttcttctcgtcgggggactgactcagacccccgggggactcggacagactcctcttgttactgtcgttcgacgacctcagaccggactacttgtcggtcctctcgtcgaccccgttcttacactcgtcgtccctctagtggtcctggtgggacgtcagactggtcctcctctaactgatactactgtggtagagacacctctacttcttcctcctgaaactgtagatgctgctcctgctcttggtctcggggtcctcgaaggtcttcttctggtccgtgatgaagtaacgacgacacctctccgacaccctgataccgtactcgtcgtcgggggtacacgactccttgtcccgggtcagaccgagacacggggtcaagttcttccaccacaaggtcctcaagtgactaccgtcgaagtgggtcggggacatgtctcccctcgacttactcgtggacccggacgacccggggatgtagtcccgactccacctcctgttgtagtaccactggaagtccttggtccggtcgtccgggatgtcgaagatgtcgtcggactagtcgatactcctcctggtctccgtcccccgactcgggtccttcttgaaacacttcgggttactttggttctggatgaagaccttccacgtcgtggtgtaccgggggtggttcctactcaaactgacgttccggacccggatgaagagactacacctggacctcttcctacacgtgagaccggactaaccgggggacgaccacacggtgtggttgtgggacttgggacgggtaccgtccgtccactgacacgtcctcaaacgggacaagaagtggtagaaactactttggttctcgaccatgaagtgactcttgtacctctccttgacgtcccgggggacgttgtaggtctacctcctggggtggaagttcctcttgatgtccaaggtacggtagttaccgatgtagtacctgtgggacggaccggaccactaccgggtcctggtctcctagtccaccatggacgactcgtacccgtcgttactcttgtaggtgtcgtaggtgaagagaccggtacacaagtgacactccttcttcctcctcatgttctaccgggacatgttggacatgggaccccacaaactctgacacctctacgacgggtcgttccgaccgtagacctcccacctcacggactaacccctcgtggacgtacgaccgtactcgtgggacaaggaccacatgtcgttgttcacggtctggggggacccgtaccggagaccggtgtagtccctgaaggtctagtgacggagaccggtcataccggtcacccgggggttcgaccggtccgacgtgatgagaccgtcgtagttacggacctcgtggttcctcgggaagtcgacctagttccacctggacgaccgggggtactagtaggtaccgtagttctgggtcccccggtccgtcttcaagtcgtcggacatgtagtcggtcaagtagtagtacatgtcggacctaccgttcttcaccgtctggatgtccccgttgtcgtgaccgtgggactaccacaagaaaccgttacacctgtcgagaccgtagttcgtgttgtagaagttgggggggtagtaacggtctatgtagtccgacgtggggtgggtgatgtcgtagtcctcgtgggactcctacctcgactacccgacactggacttgtcgacgtcgtacggggacccgtacctctcgttccggtagagactacgggtctagtgacggtcgtcgatgaagtggttgtacaaacggtggacctcggggtcgttccggtccgacgtggacgtcccgtcctcgttacggacctccggggtccagttgttggggttcctcaccgacgtccacctgaaggtcttctggtacttccactgaccccactggtgggtcccccacttctcggacgactggtcgtacatacacttcctcaaggactagtcgtcgtcggtcctaccggtggtcacctgggacaagaaggtcttaccgttccacttccacaaggtcccgttggtcctgtcgaagtggggacaccacttgtcggacctggggggggacgactggtctatggactcctaagtgggggtctcgacccacgtggtctaacgggactcctacctccacgacccgacactccgggtcctggacatgact

SEQ ID NO: 28 - DNA, 4425 bp, Artificial Sequence
  Complementary strand to FVIII transgene (V3)
tacgtctaactcgactcgtggacgaagaaggacacggacgactccaagacgaagagacggtggtcctctatgatggacccccgacacctcgactcgaccctgatgtacgtcagactggaccccctcgacggacacctacggtccaagggggggtctcacgggttctcgaaggggaagttgtggagacaccacatgttcttctgggacaaacacctcaagtgactggtggacaagttgtaacggttcgggtccggggggacctacccggacgacccggggtggtaggtccgactccacatactgtgacaccactagtgggacttcttgtaccggtcggtgggacactcggacgtacgacacccccactcgatgaccttccggagactcccccgactcatactactggtctggtcggtctccctcttcctcctactgttccacaagggacccccgtcggtgtggatacacaccgtccacgacttcctcttaccggggtaccggagactgggggacacggactggatgtcgatggactcggtacacctggaccacttcctggacttgagaccggactaaccccgggacgaccacacgtccctcccgtcggaccggttcctcttctgggtctgggacgtgttcaagtaggacgacaaacgacacaaactactcccgttctcgaccgtgagactttggttcttgtcggactacgtcctgtccctacgacggagacggtcccggaccgggttctacgtgtgacacttaccgatacacttgtcctcggacggaccggactaaccgacggtgtccttcagacacatgaccgtacactaaccgtacccgtggtggggactccacgtgtcgtagaaggacctcccggtgtggaaggaccagtccttggtgtccgtccggtcggacctctagtcggggtagtggaaggactgacgggtctgggacgactacctggacccggtcaaggacgacaagacggtgtagtcgtcggtggtcgtactaccgtacctccggatacacttccacctgtcgacgggactcctcggggtcgactcctacttcttgttactcctccgactcctgatactactactggactgactgagactctacctacaccactccaaactactactgttgtcggggtcgaagtaggtctagtccagacaccggttcttcgtggggttctggacccacgtgatgtaacgacgactcctcctcctgaccctgatacggggggaccacgaccggggactactgtcctcgatgttctcggtcatggacttgttaccgggggtctcctaaccgtccttcatgttcttccagtccaagtaccggatgtgactactttggaagttctggtccctccggtaggtcgtactcagaccgtaggacccgggggacgacatacccctccaccccctgtgggacgactagtagaagttcttggtccggtcgtccgggatgttgtagatgggggtaccgtagtgactacactccggggacatgtcgtcctccgacgggttcccccacttcgtggacttcctgaaggggtaggacggacccctctagaagttcatgttcacctgacactgacacctcctaccggggtggttcagactggggtccacggactggtctatgatgtcgtcgaaacacttgtacctctccctggaccggagaccggactaaccgggggacgactagacgatgttcctcagacacctggtctccccgttggtctagtacagactgttctccttacactaggacaagagacacaaactactcttgtcctcgaccatggactgactcttgtaggtctccaaggacgggttgggacgaccccacgtcgacctcctgggactcaaggtccggtcgttgtagtacgtgtcgtagttaccgatacacaaactgtcggacgtcgacagacacacggacgtactccaccggatgaccatgtaggactcgtaaccccgggtctgactgaaggacagacacaagaagagaccgatgtggaagttcgtgttctaccacatactcctgtgggactgggacaaggggaagagacccctctgacacaagtactcgtacctcttgggaccggacacctaagacccgacggtgttgagactgaagtccttgtccccgtactgacgggacgactttcagaggtcgacactgttcttgtgacccctgatgatactcctgtcgatactcctgtagagacggatggacgactcgttcttgttacggtaactcgggtcctcgaagtcggtcttacggtgattacacagattgttgtcgttgtggtcgttactgtcgttacacagagggggtcacgacttctccgtggtctccctctagtggtcctggtgggacgtcagactggtcctcctctaactgatactactgtggtagagacacctctacttcttcctcctgaaactgtagatgctgctcctgctcttggtctcggggtcctcgaaggtcttcttctggtccgtgatgaagtaacgacgacacctctccgacaccctgataccgtactcgtcgtcgggggtacacgactccttgtcccgggtcagaccgagacacggggtcaagttcttccaccacaaggtcctcaagtgactaccgtcgaagtgggtcggggacatgtctcccctcgacttactcgtggacccggacgacccggggatgtagtcccgactccacctcctgttgtagtaccactggaagtccttggtccggtcgtccgggatgtcgaagatgtcgtcggactagtcgatactcctcctggtctccgtcccccgactcgggtccttcttgaaacacttcgggttactttggttctggatgaagaccttccacgtcgtggtgtaccgggggtggttcctactcaaactgacgttccggacccggatgaagagactacacctggacctcttcctacacgtgagaccggactaaccgggggacgaccacacggtgtggttgtgggacttgggacgggtaccgtccgtccactgacacgtcctcaaacgggacaagaagtggtagaaactactttggttctcgaccatgaagtgactcttgtacctctccttgacgtcccgggggacgttgtaggtctacctcctggggtggaagttcctcttgatgtccaaggtacggtagttaccgatgtagtacctgtgggacggaccggaccactaccgggtcctggtctcctagtccaccatggacgactcgtacccgtcgttactcttgtaggtgtcgtaggtgaagagaccggtacacaagtgacactccttcttcctcctcatgttctaccgggacatgttggacatgggaccccacaaactctgacacctctacgacgggtcgttccgaccgtagacctcccacctcacggactaacccctcgtggacgtacgaccgtactcgtgggacaaggaccacatgtcgttgttcacggtctggggggacccgtaccggagaccggtgtagtccctgaaggtctagtgacggagaccggtcataccggtcacccgggggttcgaccggtccgacgtgatgagaccgtcgtagttacggacctcgtggttcctcgggaagtcgacctagttccacctggacgaccgggggtactagtaggtaccgtagttctgggtcccccggtccgtcttcaagtcgtcggacatgtagtcggtcaagtagtagtacatgtcggacctaccgttcttcaccgtctggatgtccccgttgtcgtgaccgtgggactaccacaagaaaccgttacacctgtcgagaccgtagttcgtgttgtagaagttgggggggtagtaacggtctatgtagtccgacgtggggtgggtgatgtcgtagtcctcgtgggactcctacctcgactacccgacactggacttgtcgacgtcgtacggggacccgtacctctcgttccggtagagactacgggtctagtgacggtcgtcgatgaagtggttgtacaaacggtggacctcggggtcgttccggtccgacgtggacgtcccgtcctcgttacggacctccggggtccagttgttggggttcctcaccgacgtccacctgaaggtcttctggtacttccactgaccccactggtgggtcccccacttctcggacgactggtcgtacatacacttcctcaaggactagtcgtcgtcggtcctaccggtggtcacctgggacaagaaggtcttaccgttccacttccacaaggtcccgttggtcctgtcgaagtggggacaccacttgtcggacctggggggggacgactggtctatggactcctaagtgggggtctcgacccacgtggtctaacgggactcctacctccacgacccgacactccgggtcctggacatgact

SEQ ID NO: 29 - protein, 1670 aa, Artificial Sequence
  FVIII polypeptide (N6)
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 30 - protein, 1474 aa, Artificial Sequence [sequence omitted]

SEQ ID NO: 31 - DNA, 1080 bp, Homo sapiens
atgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaa

SEQ ID NO: 32 - protein, 359 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 33 - DNA, 1434 bp, Homo sapiens
atgtcggctgcgcccggcctcctgcaccaggagctgtcctgcccgctgtgcctgcagctgttcgacgcgcccgtgacagccgagtgcggccacagtttctgccgcgcctgcctaggccgcgtggccggggagccggcggcggatggcaccgttctctgcccctgctgccaggcccccacgcggccgcaggcactcagcaccaacctgcagctggcgcgcctggtggaggggctggcccaggtgccgcagggccactgcgaggagcacctggacccgctgagcatctactgcgagcaggaccgcgcgctggtgtgcggagtgtgcgcctcactcggctcgcaccgcggtcatcgcctcctgcctgccgccgaggcccacgcacgcctcaagacacagctgccacagcagaaactgcagctgcaggaggcatgcatgcgcaaggagaagagtgtggctgtgctggagcatcagctggtggaggtggaggagacagtgcgtcagttccggggggccgtgggggagcagctgggcaagatgcgggtgttcctggctgcactggagggctccttggaccgcgaggcagagcgtgtacggggtgaggcaggggtcgccttgcgccgggagctggggagcctgaactcttacctggagcagctgcggcagatggagaaggtcctggaggaggtggcggacaagccgcagactgagttcctcatgaaatactgcctggtgaccagcaggctgcagaagatcctggcagagtctcccccacccgcccgtctggacatccagctgccaattatctcagatgacttcaaattccaggtgtggaggaagatgttccgggctctgatgccagcgctggaggagctgacctttgacccgagctctgcgcacccgagcctggtggtgtcttcctctggccgccgcgtggagtgctcggagcagaaggcgccgccggccggggaggacccgcgccagttcgacaaggcggtggcggtggtggcgcaccagcagctctccgagggcgagcactactgggaggtggatgttggcgacaagccgcgctgggcgctgggcgtgatcgcggccgaggccccccgccgcgggcgcctgcacgcggtgccctcgcagggcctgtggctgctggggctgcgcgagggcaagatcctggaggcacacgtggaggccaaggagccgcgcgctctgcgcagccccgagaggcggcccacgcgcattggcctttacctgagcttcggcgacggcgtcctctccttctacgatgccagcgacgccgacgcgctcgtgccgctttttgccttccacgagcgcctgcccaggcccgtgtaccccttcttcgacgtgtgctggcacgacaagggcaagaatgcccagccgctgctgctcgtgggtcccgaaggcgccgaggcctga

SEQ ID NO: 34 - protein, 477 aa, Homo sapiens
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 35 - DNA, 5115 bp, Homo sapiens
atggctgtgctcaggcagctggcgctcctcctctggaagaactacaccctgcagaagcggaaggtcctggtgacggtcctggaactcttcctgccattgctgttttctgggatcctcatctggctccgcttgaagattcagtcggaaaatgtgcccaacgccaccatctacccgggccagtccatccaggagctgcctctgttcttcaccttccctccgccaggagacacctgggagcttgcctacatcccttctcacagtgacgctgccaagaccgtcactgagacagtgcgcagggcacttgtgatcaacatgcgagtgcgcggctttccctccgagaaggactttgaggactacattaggtacgacaactgctcgtccagcgtgctggccgccgtggtcttcgagcaccccttcaaccacagcaaggagcccctgccgctggcggtgaaatatcacctacggttcagttacacacggagaaattacatgtggacccaaacaggctcctttttcctgaaagagacagaaggctggcacactacttcccttttcccgcttttcccaaacccaggaccaagggaacctacatcccctgatggcggagaacctgggtacatccgggaaggcttcctggccgtgcagcatgctgtggaccgggccatcatggagtaccatgccgatgccgccacacgccagctgttccagagactgacggtgaccatcaagaggttcccgtacccgccgttcatcgcagaccccttcctcgtggccatccagtaccagctgcccctgctgctgctgctcagcttcacctacaccgcgctcaccattgcccgtgctgtcgtgcaggagaaggaaaggaggctgaaggagtacatgcgcatgatggggctcagcagctggctgcactggagtgcctggttcctcttgttcttcctcttcctcctcatcgccgcctccttcatgaccctgctcttctgtgtcaaggtgaagccaaatgtagccgtgctgtcccgcagcgacccctccctggtgctcgccttcctgctgtgcttcgccatctctaccatctccttcagcttcatggtcagcaccttcttcagcaaagccaacatggcagcagccttcggaggcttcctctacttcttcacctacatcccctacttcttcgtggcccctcggtacaactggatgactctgagccagaagctctgctcctgcctcctgtctaatgtcgccatggcaatgggagcccagctcattgggaaatttgaggcgaaaggcatgggcatccagtggcgagacctcctgagtcccgtcaacgtggacgacgacttctgcttcgggcaggtgctggggatgctgctgctggactctgtgctctatggcctggtgacctggtacatggaggccgtcttcccagggcagttcggcgtgcctcagccctggtacttcttcatcatgccctcctattggtgtgggaagccaagggcggttgcagggaaggaggaagaagacagtgaccccgagaaagcactcagaaacgagtactttgaagccgagccagaggacctggtggcggggatcaagatcaagcacctgtccaaggtgttcagggtgggaaataaggacagggcggccgtcagagacctgaacctcaacctgtacgagggacagatcaccgtcctgctgggccacaacggtgccgggaagaccaccaccctctccatgctcacaggtctctttccccccaccagtggacgggcatacatcagcgggtatgaaatttcccaggacatggttcagatccggaagagcctgggcctgtgcccgcagcacgacatcctgtttgacaacttgacagtcgcagagcacctttatttctacgcccagctgaagggcctgtcacgtcagaagtgccctgaagaagtcaagcagatgctgcacatcatcggcctggaggacaagtggaactcacggagccgcttcctgagcgggggcatgaggcgcaagctctccatcggcatcgccctcatcgcaggctccaaggtgctgatactggacgagcccacctcgggcatggacgccatctccaggagggccatctgggatcttcttcagcggcagaaaagtgaccgcaccatcgtgctgaccacccacttcatggacgaggctgacctgctgggagaccgcatcgccatcatggccaagggggagctgcagtgctgcgggtcctcgctgttcctcaagcagaaatacggtgccggctatcacatgacgctggtgaaggagccgcactgcaacccggaagacatctcccagctggtccaccaccacgtgcccaacgccacgctggagagcagcgctggggccgagctgtctttcatccttcccagagagagcacgcacaggtttgaaggtctctttgctaaactggagaagaagcagaaagagctgggcattgccagctttggggcatccatcaccaccatggaggaagtcttccttcgggtcgggaagctggtggacagcagtatggacatccaggccatccagctccctgccctgcagtaccagcacgagaggcgcgccagcgactgggctgtggacagcaacctctgtggggccatggacccctccgacggcattggagccctcatcgaggaggagcgcaccgctgtcaagctcaacactgggctcgccctgcactgccagcaattctgggccatgttcctgaagaaggccgcatacagctggcgcgagtggaaaatggtggcggcacaggtcctggtgcctctgacctgcgtcaccctggccctcctggccatcaactactcctcggagctcttcgacgaccccatgctgaggctgaccttgggcgagtacggcagaaccgtcgtgcccttctcagttcccgggacctcccagctgggtcagcagctgtcagagcatctgaaagacgcactgcaggctgagggacaggagccccgcgaggtgctcggtgacctggaggagttcttgatcttcagggcttctgtggaggggggcggctttaatgagcggtgccttgtggcagcgtccttcagagatgtgggagagcgcacggtcgtcaacgccttgttcaacaaccaggcgtaccactctccagccactgccctggccgtcgtggacaaccttctgttcaagctgctgtgcgggcctcacgcctccattgtggtctccaacttcccccagccccggagcgccctgcaggctgccaaggaccagtttaacgagggccggaagggattcgacattgccctcaacctgctcttcgccatggcattcttggccagcacgttctccatcctggcggtcagcgagagggccgtgcaggccaagcatgtgcagtttgtgagtggagtccacgtggccagtttctggctctctgctctgctgtgggacctcatctccttcctcatccccagtctgctgctgctggtggtgtttaaggccttcgacgtgcgtgccttcacgcgggacggccacatggctgacaccctgctgctgctcctgctctacggctgggccatcatccccctcatgtacctgatgaacttcttcttcttgggggcggccactgcctacacgaggctgaccatcttcaacatcctgtcaggcatcgccaccttcctgatggtcaccatcatgcgcatcccagctgtaaaactggaagaactttccaaaaccctggatcacgtgttcctggtgctgcccaaccactgtctggggatggcagtcagcagtttctacgagaactacgagacgcggaggtactgcacctcctccgaggtcgccgcccactactgcaagaaatataacatccagtaccaggagaacttctatgcctggagcgccccgggggtcggccggtttgtggcctccatggccgcctcagggtgcgcctacctcatcctgctcttcctcatcgagaccaacctgcttcagagactcaggggcatcctctgcgccctccggaggaggcggacactgacagaattatacacccggatgcctgtgcttcctgaggaccaagatgtagcggacgagaggacccgcatcctggcccccagtccggactccctgctccacacacctctgattatcaaggagctctccaaggtgtacgagcagcgggtgcccctcctggccgtggacaggctctccctcgcggtgcagaaaggggagtgcttcggcctgctgggcttcaatggagccgggaagaccacgactttcaaaatgctgaccggggaggagagcctcacttctggggatgcctttgtcgggggtcacagaatcagctctgatgtcggaaaggtgcggcagcggatcggctactgcccgcagtttgatgccttgctggaccacatgacaggccgggagatgctggtcatgtacgctcggctccggggcatccctgagcgccacatcggggcctgcgtggagaacactctgcggggcctgctgctggagccacatgccaacaagctggtcaggacgtacagtggtggtaacaagcggaagctgagcaccggcatcgccctgatcggagagcctgctgtcatcttcctggacgagccgtccactggcatggaccccgtggcccggcgcctgctttgggacaccgtggcacgagcccgagagtctggcaaggccatcatcatcacctcccacagcatggaggagtgtgaggccctgtgcacccggctggccatcatggtgcaggggcagttcaagtgcctgggcagcccccagcacctcaagagcaagttcggcagcggctactccctgcgggccaaggtgcagagtgaagggcaacaggaggcgctggaggagttcaaggccttcgtggacctgacctttccaggcagcgtcctggaagatgagcaccaaggcatggtccattaccacctgccgggccgtgacctcagctgggcgaaggttttcggtattctggagaaagccaaggaaaagtacggcgtggacgactactccgtgagccagatctcgctggaacaggtcttcctgagcttcgcccacctgcagccgcccaccgcagaggaggggcgatga

SEQ ID NO: 36 - protein, 1704 aa, Homo sapiens
MAVLRQLALLLWKNYTLQKRKVLVTVLELFLPLLFSGILIWLRLKIQSENVPNATIYPGQSIQELPLFFTFPPPGDTWELAYIPSHSDAAKTVTETVRRALVINMRVRGFPSEKDFEDYIRYDNCSSSVLAAVVFEHPFNHSKEPLPLAVKYHLRFSYTRRNYMWTQTGSFFLKETEGWHTTSLFPLFPNPGPREPTSPDGGEPGYIREGFLAVQHAVDRAIMEYHADAATRQLFQRLTVTIKRFPYPPFIADPFLVAIQYQLPLLLLLSFTYTALTIARAVVQEKERRLKEYMRMMGLSSWLHWSAWFLLFFLFLLIAASFMTLLFCVKVKPNVAVLSRSDPSLVLAFLLCFAISTISFSFMVSTFFSKANMAAAFGGFLYFFTYIPYFFVAPRYNWMTLSQKLCSCLLSNVAMAMGAQLIGKFEAKGMGIQWRDLLSPVNVDDDFCFGQVLGMLLLDSVLYGLVTWYMEAVFPGQFGVPQPWYFFIMPSYWCGKPRAVAGKEEEDSDPEKALRNEYFEAEPEDLVAGIKIKHLSKVFRVGNKDRAAVRDLNLNLYEGQITVLLGHNGAGKTTTLSMLTGLFPPTSGRAYISGYEISQDMVQIRKSLGLCPQHDILFDNLTVAEHLYFYAQLKGLSRQKCPEEVKQMLHIIGLEDKWNSRSRFLSGGMRRKLSIGIALIAGSKVLILDEPTSGMDAISRRAIWDLLQRQKSDRTIVLTTHFMDEADLLGDRIAIMAKGELQCCGSSLFLKQKYGAGYHMTLVKEPHCNPEDISQLVHHHVPNATLESSAGAELSFILPRESTHRFEGLFAKLEKKQKELGIASFGASITTMEEVFLRVGKLVDSSMDIQAIQLPALQYQHERRASDWAVDSNLCGAMDPSDGIGALIEEERTAVKLNTGLALHCQQFWAMFLKKAAYSWREWKMVAAQVLVPLTCVTLALLAINYSSELFDDPMLRLTLGEYGRTVVPFSVPGTSQLGQQLSEHLKDALQAEGQEPREVLGDLEEFLIFRASVEGGGFNERCLVAASFRDVGERTVVNALFNNQAYHSPATALAVVDNLLFKLLCGPHASIVVSNFPQPRSALQAAKDQFNEGRKGFDIALNLLFAMAFLASTFSILAVSERAVQAKHVQFVSGVHVASFWLSALLWDLISFLIPSLLLLVVFKAFDVRAFTRDGHMADTLLLLLLYGWAIIPLMYLMNFFFLGAATAYTRLTIFNILSGIATFLMVTIMRIPAVKLEELSKTLDHVFLVLPNHCLGMAVSSFYENYETRRYCTSSEVAAHYCKKYNIQYQENFYAWSAPGVGRFVASMAASGCAYLILLFLIETNLLQRLRGILCALRRRRTLTELYTRMPVLPEDQDVADERTRILAPSPDSLLHTPLIIKELSKVYEQRVPLLAVDRLSLAVQKGECFGLLGFNGAGKTTTFKMLTGEESLTSGDAFVGGHRISSDVGKVRQRIGYCPQFDALLDHMTGREMLVMYARLRGIPERHIGACVENTLRGLLLEPHANKLVRTYSGGNKRKLSTGIALIGEPAVIFLDEPSTGMDPVARRLLWDTVARARESGKAIIITSHSMEECEALCTRLAIMVQGQFKCLGSPQHLKSKFGSGYSLRAKVQSEGQQEALEEFKAFVDLTFPGSVLEDEHQGMVHYHLPGRDLSWAKVFGILEKAKEKYGVDDYSVSQISLEQVFLSFAHLQPPTAEEGR

SEQ ID NO: 37 - DNA, 600 bp, Woodchuck hepatitis virus
gggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagct